SEQUENCE LISTING (19 sequences)

SEQ ID NO: 1 - DNA, 1503 bp, Homo sapiens
cgggctgaatggaatgagccggattggcggagccgggctgcccgcagccgcgctcaccggcccgggacgcttccgcatggcccgcgaggagccggcgcccgcggcgttggcggctgccgggcagcccgggggcggcagaggcggcgagcgggcgctgcaggggccaggggtcgcccgcagggggcggccatcgctgagccgcgctaaactgcacgggctgcggcacatgtgtgccgggcgcacggcggctgggggctccttccagcggcgggcgctgtgggtgctggccttctgtacatccttcggcttgctgctgtcctggtcctcgaaccgcttgctctactggctcagcttcccgtcacacacgcgggtgcaccgcgagtggagccgccagttacccttccccgccgtcactgtgtgcaacaacaacccgctgcgcttcccgcgcctctccaagggggacctctactatgccggccactggctcgggctgctgctgcccaaccgcaccgcgcgcccgcttgtcagcgagctgctgcggggcgacgagccgcgccgccagtggttccgcaagctggcggacttccgcctcttcctgcctccgcgccacttcgagggaatcagcgccgccttcatggaccgcctgggccaccagctggaggacatgctgctctcctgcaagtaccgcggcgagctctgcgggccgcacaacttctcctcgctcacatacctgcccccaccgtggggtgagtgccgatcctcagagatgggcctcgacttttttcctgtttacagcatcaccgcctgtaggattgactgtgagacccgctacattgtggaaaactgcaactgccgcatggttcacatgccaggggatgcccctttttgtacccctgagcagcacaaggagtgtgcagagcctgccctaggtctggcggaaaaggacagcaattactgtctctgcaggacaccctgcaacctaacccgctacaacaaagagctctccatggtgaagatccccagcaagacatcagccaagtaccttgagaagaaatttaacaaatcagaaaaatatatctcagagaacatccttgttctggatatattttttgaagctctcaattatgagacaattgaacagaagaaggcgtatgaagttgctgccttacttggtgatattggtggtcagatgggattgttcattggtgctagtatccttacaatactagagctctttgattatatttatgagctgatcaaagagaagctattagacctgcttggcaaagaggaggacgaagggagccacgatgagaatgtgacacaatgccaaaccactctgaaaccatcagtcacactgtgaacgtgcccctgcagacgaccctggggaccttggaggagattgcctgctgacacccctcgagtcacccagcactccctccaaacagaccttgaggcccaagacccaggacaaggaacagcaagctcaggtgggatggcccc

SEQ ID NO: 2 - DNA, 1948 bp, Rattus norvegicus
cctcgggctgaatgaatgagccggagcggcggagcccggctgcccgcgaccgcgctcagcggcccgggacgcttccgtatggcccgcgagcagccggcgcccgtggcggtggcggcagctaggcagcccggaggagaccggagcggcgatccggcgctgcaggggccaggggtcgcccgcagggggcggccgtccctgagtcgcactaaattgcacgggctgcggcacatgtgcgcggggcgcacggcggcgggaggctctttccagcgacgggcgctgtgggtgctggccttctgcacgtccctcggcttgctgctgtcctggtcctcgaaccgcctgctctactggctcagcttcccgtcacacacacgagtgcaccgtgagtggagccgccagctgccgttccccgccgtcaccgtgtgcaacaacaaccccctgcgcttcccgcgcctctccaagggggacctctactacgcgggccactggctagggctgctgcttcccaaccgcaccgcgcgcccgctggtcagcgagctgctgcggggcgacgagccgcgccgccagtggttccgcaaactggccgacttccgcctcttcctgccgccgcgccacttcgagggcatcagcgctgccttcatggaccgtttgggccaccagctggaggatatgctgctctcctgcaagtaccggggcgagctctgtggcccgcacaacttctcctcagtgtttacaaaatacgggaagtgttacatgtttaactcaggcgaggatggcaagccgctgctcaccacggtcaagggggggacgggcaacgggctggagatcatgctggacattcagcaagatgagtacctgcccatctggggagagacagaggaaacaacgtttgaagcaggagtgaaggttcagatccacagtcagtctgagccgcctttcatccaagagctgggctttggggtggctccggggttccagaccttcgtggccacacaagagcagaggctcacatatctgcccccaccatggggggagtgccggtcctcagagatgggactcgacttctttcctgtttacagcatcacagcctgtcggattgactgtgagacccgctacatcgtggagaactgtaactgccgcatggtccacatgccaggggacgcccctttctgcacccctgagcagcacaaggagtgtgcagagcctgccctcggtctactggcagaaaaggacagcaattactgtctctgcaggacaccctgcaacctgacacgctacaacaaagagctctccatggtgaagatccccagcaagacgtcagccaagtacttagagaagaaatttaacaaatcggaaaaatatatctcagagaacattcttgttctggacatattttttgaggcgctcaattacgaaacaattgaacagaagaaggcgtatgaagttgctgccttacttggtgacatcggtggtcagatgggactgttcattggtgctagtctcctcacaatactagagctctttgattatatttatgagctgatcaaagagaagctattagacctgcttggcaaagaagaagaggaagggagccacgatgagaacatgagcacctgtgacacaatgccaaaccactctgaaaccatcagccacactgtgaacgtgcccctgcagacagctttgggcaccctggaggagattgcctgctgacacctctcaggcaacgcagcacctccaaacagaccttaaaggcccaagacctaggacaggagacagcaagcgcaggtgggatcgcccctgacgactgaaagaagcagagccccccatatgcacacattgcgaacttctgccaaacctcacctggccacatctgacatgaaccgtcccgggccctgcgtcatgtccctcgcaggaccgatgagtcgcactccggaactgtccaagaactaac

SEQ ID NO: 3 - protein, 563 aa, Rattus norvegicus
MSRSGGARLPATALSGPGRFRMAREQPAPVAVAAARQPGGDRSGDPALQGPGVARRGRPSLSRTKLHGLRHMCAGRTAAGGSFQRRALWVLAFCTSLGLLLSWSSNRLLYWLSFPSHTRVHREWSRQLPFPAVTVCNNNPLRFPRLSKGDLYYAGHWLGLLLPNRTARPLVSELLRGDEPRRQWFRKLADFRLFLPPRHFEGISAAFMDRLGHQLEDMLLSCKYRGELCGPHNFSSVFTKYGKCYMFNSGEDGKPLLTTVKGGTGNGLEIMLDIQQDEYLPIWGETEETTFEAGVKVQIHSQSEPPFIQELGFGVAPGFQTFVATQEQRLTYLPPPWGECRSSEMGLDFFPVYSITACRIDCETRYIVENCNCRMVHMPGDAPFCTPEQHKECAEPALGLLAEKDSNYCLCRTPCNLTRYNKELSMVKIPSKTSAKYLEKKFNKSEKYISENILVLDIFFEALNYETIEQKKAYEVAALLGDIGGQMGLFIGASLLTILELFDYIYELIKEKLLDLLGKEEEEGSHDENMSTCDTMPNHSETISHTVNVPLQTALGTLEEIAC

SEQ ID NO: 4 - DNA, 2747 bp, Homo sapiens
ctcttccacatcaccttggtgtctccctaaataaaaccagccctccttatcgcctggaaaaaatcaagagctagagtttgaatgggttttatataaacactcacccctgtcagcgtgcggctgggctctcaggataaactcacagcatctggcgcgatgcttgccttgcgttctctcccctgaacgtcaaggtttaagcagagcccgaggactgggagctcttctctgaaattcgatcaacctgaagccagttgcggaactgcacggggtcccgatggacctcaaggaaagccccagtgagggcagcctgcaaccttctagcatccagatctttgccaacacctccaccctccatggcatccgccacatcttcgtgtatgggccgctgaccatccggcgtgtgctgtgggcagtggccttcgtgggctctctgggcctgctgctggtggagagctctgagagggtgtcctactacttctcctaccagcatgtcactaaggtggacgaagtggtggctcaaagcctggtcttcccagctgtgaccctctgtaacctgaatggcttccggttctccaggctcaccaccaacgacctgtaccatgctggggagctgctggccctgctggatgtcaacctgcagatcccggacccccatctggctgacccctccgtgctggaggccctgcggcagaaggccaacttcaagcactacaaacccaagcagttcagcatgctggagttcctgcaccgtgtgggccatgacctgaaggatatgatgctctactgcaagttcaaagggcaggagtgcggccaccaagacttcaccacagtgtttacaaaatatgggaagtgttacatgtttaactcaggcgaggatggcaaacctctgctcaccacggtcaagggggggacaggcaacgggctggagatcatgctggacattcagcaggatgagtacctgcccatctggggagagacagaggaaacgacatttgaagcaggagtgaaagttcagatccacagtcagtctgagccacctttcatccaagagctgggctttggggtggctccagggttccagacctttgtggccacacaggagcagaggctcacatacctgcccccaccgtggggtgagtgccgatcctcagagatgggcctcgacttttttcctgtttacagcatcaccgcctgtaggattgactgtgagacccgctacattgtggaaaactgcaactgccgcatggttcacatgccaggggatgcccctttttgtacccctgagcagcacaaggagtgtgcagagcctgccctaggtctgttggcggaaaaggacagcaattactgtctctgcaggacaccctgcaacctaacccgctacaacaaagagctctccatggtgaagatccccagcaagacatcagccaagtaccttgagaagaaatttaacaaatcagaaaaatatatctcagagaacatccttgttctggatatattttttgaagctctcaattatgagacaattgaacagaagaaggcgtatgaagttgctgccttacttggtgatattggtggtcagatgggattgttcattggtgctagtatccttacaatactagagctctttgattatatttatgagctgatcaaagagaagctattagacctgcttggcaaagaggaggacgaagggagccacgatgagaatgtgagtacttgtgacacaatgccaaaccactctgaaaccatcagtcacactgtgaacgtgcccctgcagacgaccctggggaccttggaggagattgcctgctgacacccctcgagtcacccagcactccctccaaacagaccttgaggcccaagacccaggacaaggaacagcaagctcaggtgggatggccccagtgctggaaagaagcaagagccccctatgcacacattgcagactagctgcctagacctcgctccggccacgtccaacacgacgcatccttgggccccgccgtgcgtccctcttaggagagatgagtcacactctggaactgtccaagaacgaacctgccatcacatctcactgccagatgtataaagcacctgcatgctcagacttcttgtggcgccacctccacgtctgtcttgtacatgacactcctccacgcggtttccagtgtccacactgctgcccgtgcagtgggaccagattccaggtccaaagtcaccatgaggccaccctggaatcagaactgcacaatcaagagggaacccatgggactctctgctacattcagttcttgtgtcgtttgtgaaagttcttaacctgcccaaaaacccccttttccccaagctgcccatggggcttcggcgccaaaggtgacccgcgccaacctccctcccccccagtgcctatgacggcggcacagcagccagcgggtgggggacgcctgtgttcacccatggtgcccatgtcgttcttctctccctgtgacacagcttgtacagtctgattctttttatctggggtaggggggcttttatgtttgtccgatggagatttgttttgttttgcttcattttatgcttttttattttagttttgatgttctgaggtttgctttggtttttccattttctttggcatttatttattcgtgcttcaaatcacagtcatattaaaagctggtcttgtggaaaaaaaaaaaaaaaaaaaaaaaaaaaaaaaaaaaaaaaaaaaaaaaaaaaaaaaaaaa

SEQ ID NO: 5 - protein, 512 aa, Homo sapiens
MDLKESPSEGSLQPSSIQIFANTSTLHGIRHIFVYGPLTIRRVLWAVAFVGSLGLLLVESSERVSYYFSYQHVTKVDEVVAQSLVFPAVTLCNLNGFRFSRLTTNDLYHAGELLALLDVNLQIPDPHLADPSVLEALRQKANFKHYKPKQFSMLEFLHRVGHDLKDMMLYCKFKGQECGHQDFTTVFTKYGKCYMFNSGEDGKPLLTTVKGGTGNGLEIMLDIQQDEYLPIWGETEETTFEAGVKVQIHSQSEPPFIQELGFGVAPGFQTFVATQEQRLTYLPPPWGECRSSEMGLDFFPVYSITACRIDCETRYIVENCNCRMVHMPGDAPFCTPEQHKECAEPALGLLAEKDSNYCLCRTPCNLTRYNKELSMVKIPSKTSAKYLEKKFNKSEKYISENILVLDIFFEALNYETIEQKKAYEVAALLGDIGGQMGLFIGASILTILELFDYIYELIKEKLLDLLGKEEDEGSHDENVSTCDTMPNHSETISHTVNVPLQTTLGTLEEIAC

SEQ ID NO: 6 - DNA, 1702 bp, Homo sapiens
gaatgagccggattggcggagccgggctgcccgcagccgcgctcaccggcccgggacgcttccgcatggcccgcgaggagccggcgcccgcggcgttggcggctgccgggcagcccgggggcggcagaggcggcgagctggcgctgcaggggccaggggtcgcccgcagggggcggccatcgctgagccgcgctaaactgcacgggctgcggcacatgtgtgccgggcgcacggcggctgggggctccttccagcggcgggcgctgtgggtgctggccttctgtacatccttcggcttgctgctgccctggtcctcgaaccgcttgctctactggctcagcttcccgtcacacacgcgggtgcaccgcgagtggagccgccagttacccttccccgccgtcaccgtgtgcaacaacaacccgctgcgcttcccgcgcctctccaagggggacctctactatgccggccactggctcgggctgctgctgcccaaccgcaccgcgcgctcgcttgtcagcgagctgctgcggggcgacgagccgcgccgccagtggttccgcaagctggcggacttccgcctctccctgcctccgcgccacttcgagggaatcagcgccgccttcatggaccgcctgggccaccagctggaggacatgctgctctcctgcaagtaccgcggcgagctctgcgggccgcacaacttctcctccgtgtttacaaaatatgggaagtgttacatgtttaactcaggcgaggatggcaaacctctgctcaccacggtcaagggggggacaggcaacgggctggagatcatgctggacattcagcaggatgagtacctgcccatctggggagagacagaggaaacgacatttgaagcaggagtgaaagttcagatccacagtcagtctgagccacctttcatccaagagctgggctttggggtggctccagggttccagacctttgtggccacacaggagcagaggctcacatacctgcccccaccgtggggtgagtgccgatcctcagagatgggcctcgacttttttcctgtttacagcatcaccgcctgtaggattgactgtgagacccgctacattgtggaaaactgcaactgccgcatggttcacatgccaggggatgcccctttttgtacccctgagcagcacaaggagtgtgcagagcctgccctaggtctgttggcggaaaaggacagcaattactgtctctgcaggacaccctgcaacctaacccgctacaacaaagagctctccatggtgaagatccccagcaagacatcagccaagtaccttgagaagaaatttaacaaatcagaaaaatatatctcagagaacatccttgttctggatatattttttgaagctctcaattatgagacaattgaacagaagaaggcgtatgaagttgctgccttacttggtgatattggtggtcagatgggattgttcattggtgctagtatccttacaatactagagctctttgattatatttatgagctgatcaaagagaagctattagacctgcttggcaaagaggaggacgaagggagccacgatgagaatgtgagtacttgtgacacaatgccaaaccactctgaaaccatcagtcacactgtgaacgtgcccctgcagacgaccctggggaccttggaggagattgcctgctgacacccctc

SEQ ID NO: 7 - protein, 563 aa, Homo sapiens
MSRIGGAGLPAAALTGPGRFRMAREEPAPAALAAAGQPGGGRGGELALQGPGVARRGRPSLSRAKLHGLRHMCAGRTAAGGSFQRRALWVLAFCTSFGLLLPWSSNRLLYWLSFPSHTRVHREWSRQLPFPAVTVCNNNPLRFPRLSKGDLYYAGHWLGLLLPNRTARSLVSELLRGDEPRRQWFRKLADFRLSLPPRHFEGISAAFMDRLGHQLEDMLLSCKYRGELCGPHNFSSVFTKYGKCYMFNSGEDGKPLLTTVKGGTGNGLEIMLDIQQDEYLPIWGETEETTFEAGVKVQIHSQSEPPFIQELGFGVAPGFQTFVATQEQRLTYLPPPWGECRSSEMGLDFFPVYSITACRIDCETRYIVENCNCRMVHMPGDAPFCTPEQHKECAEPALGLLAEKDSNYCLCRTPCNLTRYNKELSMVKIPSKTSAKYLEKKFNKSEKYISENILVLDIFFEALNYETIEQKKAYEVAALLGDIGGQMGLFIGASILTILELFDYIYELIKEKLLDLLGKEEDEGSHDENVSTCDTMPNHSETISHTVNVPLQTTLGTLEEIAC

SEQ ID NO: 8 - DNA, 1702 bp, Homo sapiens
gaatgagccggattggcggagccgggctgcccgcagccgcgctcaccggcccgggacgcttccgcatggcccgcgaggagccggcgcccgcggcgttggcggctgccgggcagcctgggggcggcagaggcggcgagcgggcgctgcaggggccaggggtcgcccgcagggggcggccatcgctgagccgcgctaaactgcacgggctgcggcacatgtgtgccgggcgcacggcggctgggggctccttccagcggcgggcgctgtgggtgctggccttctgtacatccttcggcttgctgctgtcctggtcctcgaaccgcttgctctactggctcagcttcccatcacacacgcgggtgcaccgcgagtggagccgccagttacccttccccgccgtcactgtgtgcaacaacaacccgctgcgcttcccgcgcctctccaagggggacctctactatgccggccactggctcgggctgctgctgcccaaccgcaccgcgcgcccgcttgtcagcgagctgctgcggggcgacgagccgcgccgccagtggttccgcaagctggcggacttccgcctcttcctgcctccgcgccacttcgggggaatcagcgccgccttcatggaccgcctgggccaccagctggaggacatgctgctctcctgcaagtaccgcggcgagctctgcgggccgcacaacttctcctccgtgtttacaaaatatgggaagtgttacatgtttaactcaggcgaggatggcaaacctctgctcaccacggtcaagggggggacaggcaacgggctggagatcatgctggacattcagcaggatgagtacctgcccatctggggagagacaaaggaaacgacatttgaagcaggagtgaaagttcagatccacagtcagtctgagccacctttcatccaagagctgggctttggggtggctccagggttccagacctttgtggccacacaggagcagaggctcacatacctgcccccaccgtggggtgagtgccgatcctcagagatgggcctcgacttttttcctgtttacagcatcaccgcctgtaggattgactgtgagacccgctacattgtggaaaactgcaactgccgcatggttcacatgccaggggatgcccctttttgtacccctgagcagcacaaggagtgtgcagagcctgccctaggtctgttggcggaaaaggacagcaattactgtctctgcaggacaccctgcaacctaacccgctacaacaaagagctctccatggtgaagatccccagcaagacatcagccaagtaccttgagaagaaatttaacaaatcagaaaaatatatctcagagaacatccttgttctggatatattttttgaagctctcaattatgagacaattgaacagaagaaggcgtatgaagttgctgccttacttggtgatattggtggtcagatgggattgttcattggtgctagtatccttacaatactagagctctttggttatatttatgagctgatcaaagagaagctattagacctgcttggcaaagaggaggacgaagggagtcacgatgagaatgtgagtacttgtgacacaatgccaaaccactctgaaaccatcagtcacactgtgaacgtgcccctgcagacgaccctggggaccttggaggagattgcctgctgacacccctc

SEQ ID NO: 9 - protein, 563 aa, Homo sapiens
MSRIGGAGLPAAALTGPGRFRMAREEPAPAALAAAGQPGGGRGGERALQGPGVARRGRPSLSRAKLHGLRHMCAGRTAAGGSFQRRALWVLAFCTSFGLLLSWSSNRLLYWLSFPSHTRVHREWSRQLPFPAVTVCNNNPLRFPRLSKGDLYYAGHWLGLLLPNRTARPLVSELLRGDEPRRQWFRKLADFRLFLPPRHFGGISAAFMDRLGHQLEDMLLSCKYRGELCGPHNFSSVFTKYGKCYMFNSGEDGKPLLTTVKGGTGNGLEIMLDIQQDEYLPIWGETKETTFEAGVKVQIHSQSEPPFIQELGFGVAPGFQTFVATQEQRLTYLPPPWGECRSSEMGLDFFPVYSITACRIDCETRYIVENCNCRMVHMPGDAPFCTPEQHKECAEPALGLLAEKDSNYCLCRTPCNLTRYNKELSMVKIPSKTSAKYLEKKFNKSEKYISENILVLDIFFEALNYETIEQKKAYEVAALLGDIGGQMGLFIGASILTILELFGYIYELIKEKLLDLLGKEEDEGSHDENVSTCDTMPNHSETISHTVNVPLQTTLGTLEEIAC

SEQ ID NO: 10 - DNA, 1703 bp, Homo sapiens
ggaatgagccggattggcggagccgggctgcccgcagccgcgctcaccggcccgggacgcttccgcatggcccgcgaggagccggcgcccgcggcgttggcggctgccgggcagcccgggggcggcagaggcggcgagctggcgctgcaggggccaggggtcgcccgcagggggcggccatcgctgagccgcgctaaactgcacgggctgcggcacatgtgtgccgggcgcacggcggctgggggctccttccagcggcgggcgctgtgggtgctggccttctgtacatccttcggcttgctgctgtcctggtcctcgaaccgcttgctctactggctcagcttcccgtcacacacgcgggtgcaccgcgagtggagccgccagttacccttccccgccgtcaccgtgtgcaacaacaacccgctgcgcttcccgcgcctctccaagggggacctctactatgccggccactggctcgggctgctgctgcccaaccgcaccgcgcgtccgcttgtcagcgagctgctgcggggcgacgagccgcgccgccagtggttccgcaagctggcggacttccgcctcttcctgcctccgcgccacttcgagggaatcagcgccgccttcatggaccgcctgggccaccagctggaggacatgctgctctcctgcaagtaccgcggcgagctctgcgggccgcacaacttctcctccgtgtttacaaaatatgggaagtgttacatgtttaactcaggcgaggatggcaaacctctgctcaccacggtcaagggggggacaggcaacgggctggagatcatgctggacattcagcaggatgagtacctgcccatctggggagagacagaggaaacgacatttgaagcaggagtgaaagttcagatccacagtcagtctgagccacctttcatccaagagctgggctttggggtggctccagggttccagacctttgtggccacacaggagcagaggctcacatacctgcccccaccgtggggtgagtgccgatcctcagagatgggcctcgacttttttcctgtttacagcatcaccgcctgtaggattgactgtgagacccgctacattgtggaaaactgcaactgccgcatggttcacatgccaggggatgcccctttttgtacccctgagcagcacaaggagtgtgcagagcctgccctaggtctgttggcggaaaaggacagcaattactgtctctgcaggacaccctgcaacctaacccgctacaacaaagagctctccatggtgaagatccccagcaagacatcagccaagtaccttgagaagaaatttaacaaatcagaaaaatatatctcagagaacatccttgttctggatatattttttgaagctctcaattatgagacaattgaacagaagaaggcgtatgaagttgctgccttacttggtgatattggtggtcagatgggattgttcattggtgctagtatccttacaatactagagctctttgattatatttatgagctgatcaaagagaagctattagacctgcttggcaaagaggaggacgaagggagccacgatgagaatgtgagtacttgtgacacaatgccaaaccactctgaaaccatcagtcacactgtgaacgtgcccctgcagacgaccctggggaccttggaggagattgcctgctgacacccctc

SEQ ID NO: 11 - protein, 563 aa, Homo sapiens
MSRIGGAGLPAAALTGPGRFRMAREEPAPAALAAAGQPGGGRGGERALQGPGVARRGRPSLSRAKLHGLRHMCAGRTAAGGSFQRRALWVLAFCTSFGLLLSWSSNRLLYWLSFPSHTRVHREWSRQLPFPAVTVCNNNPLRFPRLSKGDLYYAGHWLGLLLPNRTARPLVSELLRGDEPRRQWFRKLADFRLFLPPRHFEGISAAFMDRLGHQLEDMLLSCKYRGELCGPHNFSSVFTKYGKCYMFNSGEDGKPLLTTVKGGTGNGLEIMLDIQQDEYLPIWGETEETTFEAGVKVQIHSQSEPPFIQELGFGVAPGFQTFVATQEQRLTYLPPPWGECRSSEMGLDFFPVYSITACRIDCETRYIVENCNCRMVHMPGDAPFCTPEQHKECAEPALGLLAEKDSNYCLCRTPCNLTRYNKELSMVKIPSKTSAKYLEKKFNKSEKYISENILVLDIFFEALNYETIEQKKAYEVAALLGDMGGQMGLFIGASILTILELFDYIYELIKEKLLDLLGKEEDEGSHDENVSTCDTMPNHSETISHTVNVPLQTTLGTLEEIAC

SEQ ID NO: 12 - DNA, 22 bp, Artificial
  Primer sequence
ngaatgagccggattggcggag

SEQ ID NO: 13 - DNA, 23 bp, Artificial
  Primer sequence
gaggggtgtcagcaggcaatctc

SEQ ID NO: 14 - protein, 563 aa, Homo sapiens
  MISC_FEATURE (46)..(46): Xaa is Arg or Leu
MSRIGGAGLPAAALTGPGRFRMAREEPAPAALAAAGQPGGGRGGEXALQGPGVARRGRPSLSRAKLHGLRHMCAGRTAAGGSFQRRALWVLAFCTSFGLLLXWSSNRLLYWLSFPSHTRVHREWSRQLPFPAVTVCNNNPLRFPRLSKGDLYYAGHWLGLLLPNRTARXLVSELLRGDEPRRQWFRKLADFRLXLPPRHFXGISAAFMDRLGHQLEDMLLSCKYRGELCGPHNFSSVFTKYGKCYMFNSGEDGKPLLTTVKGGTGNGLEIMLDIQQDEYLPIWGETXETTFEAGVKVQIHSQSEPPFIQELGFGVAPGFQTFVATQEQRLTYLPPPWGECRSSEMGLDFFPVYSITACRIDCETRYIVENCNCRMVHMPGDAPFCTPEQHKECAEPALGLLAEKDSNYCLCRTPCNLTRYNKELSMVKIPSKTSAKYLEKKFNKSEKYISENILVLDIFFEALNYETIEQKKAYEVAALLGDXGGQMGLFIGASILTILELFXYIYELIKEKLLDLLGKEEDEGSHDENVSTCDTMPNHSETISHTVNVPLQTTLGTLEEIAC

SEQ ID NO: 15 - DNA, 1689 bp, Homo sapiens
atgagccggattggcggagccgggctgcccgcagccgcgctcaccggcccgggacgcttccgcatggcccgcgaggagccggcgcccgcggcgttggcggctgccgggcagcccgggggcggcagaggcggcgagctggcgctgcaggggccaggggtcgcccgcagggggcggccatcgctgagccgcgctaaactgcacgggctgcggcacatgtgtgccgggcgcacggcggctgggggctccttccagcggcgggcgctgtgggtgctggccttctgtacatccttcggcttgctgctgccctggtcctcgaaccgcttgctctactggctcagcttcccgtcacacacgcgggtgcaccgcgagtggagccgccagttacccttccccgccgtcaccgtgtgcaacaacaacccgctgcgcttcccgcgcctctccaagggggacctctactatgccggccactggctcgggctgctgctgcccaaccgcaccgcgcgctcgcttgtcagcgagctgctgcggggcgacgagccgcgccgccagtggttccgcaagctggcggacttccgcctctccctgcctccgcgccacttcgagggaatcagcgccgccttcatggaccgcctgggccaccagctggaggacatgctgctctcctgcaagtaccgcggcgagctctgcgggccgcacaacttctcctccgtgtttacaaaatatgggaagtgttacatgtttaactcaggcgaggatggcaaacctctgctcaccacggtcaagggggggacaggcaacgggctggagatcatgctggacattcagcaggatgagtacctgcccatctggggagagacagaggaaacgacatttgaagcaggagtgaaagttcagatccacagtcagtctgagccacctttcatccaagagctgggctttggggtggctccagggttccagacctttgtggccacacaggagcagaggctcacatacctgcccccaccgtggggtgagtgccgatcctcagagatgggcctcgacttttttcctgtttacagcatcaccgcctgtaggattgactgtgagacccgctacattgtggaaaactgcaactgccgcatggttcacatgccaggggatgcccctttttgtacccctgagcagcacaaggagtgtgcagagcctgccctaggtctgttggcggaaaaggacagcaattactgtctctgcaggacaccctgcaacctaacccgctacaacaaagagctctccatggtgaagatccccagcaagacatcagccaagtaccttgagaagaaatttaacaaatcagaaaaatatatctcagagaacatccttgttctggatatattttttgaagctctcaattatgagacaattgaacagaagaaggcgtatgaagttgctgccttacttggtgatattggtggtcagatgggattgttcattggtgctagtatccttacaatactagagctctttgattatatttatgagctgatcaaagagaagctattagacctgcttggcaaagaggaggacgaagggagccacgatgagaatgtgagtacttgtgacacaatgccaaaccactctgaaaccatcagtcacactgtgaacgtgcccctgcagacgaccctggggaccttggaggagattgcctgc

SEQ ID NO: 16 - DNA, 1689 bp, Homo sapiens
atgagccggattggcggagccgggctgcccgcagccgcgctcaccggcccgggacgcttccgcatggcccgcgaggagccggcgcccgcggcgttggcggctgccgggcagcctgggggcggcagaggcggcgagcgggcgctgcaggggccaggggtcgcccgcagggggcggccatcgctgagccgcgctaaactgcacgggctgcggcacatgtgtgccgggcgcacggcggctgggggctccttccagcggcgggcgctgtgggtgctggccttctgtacatccttcggcttgctgctgtcctggtcctcgaaccgcttgctctactggctcagcttcccatcacacacgcgggtgcaccgcgagtggagccgccagttacccttccccgccgtcactgtgtgcaacaacaacccgctgcgcttcccgcgcctctccaagggggacctctactatgccggccactggctcgggctgctgctgcccaaccgcaccgcgcgcccgcttgtcagcgagctgctgcggggcgacgagccgcgccgccagtggttccgcaagctggcggacttccgcctcttcctgcctccgcgccacttcgggggaatcagcgccgccttcatggaccgcctgggccaccagctggaggacatgctgctctcctgcaagtaccgcggcgagctctgcgggccgcacaacttctcctccgtgtttacaaaatatgggaagtgttacatgtttaactcaggcgaggatggcaaacctctgctcaccacggtcaagggggggacaggcaacgggctggagatcatgctggacattcagcaggatgagtacctgcccatctggggagagacaaaggaaacgacatttgaagcaggagtgaaagttcagatccacagtcagtctgagccacctttcatccaagagctgggctttggggtggctccagggttccagacctttgtggccacacaggagcagaggctcacatacctgcccccaccgtggggtgagtgccgatcctcagagatgggcctcgacttttttcctgtttacagcatcaccgcctgtaggattgactgtgagacccgctacattgtggaaaactgcaactgccgcatggttcacatgccaggggatgcccctttttgtacccctgagcagcacaaggagtgtgcagagcctgccctaggtctgttggcggaaaaggacagcaattactgtctctgcaggacaccctgcaacctaacccgctacaacaaagagctctccatggtgaagatccccagcaagacatcagccaagtaccttgagaagaaatttaacaaatcagaaaaatatatctcagagaacatccttgttctggatatattttttgaagctctcaattatgagacaattgaacagaagaaggcgtatgaagttgctgccttacttggtgatattggtggtcagatgggattgttcattggtgctagtatccttacaatactagagctctttggttatatttatgagctgatcaaagagaagctattagacctgcttggcaaagaggaggacgaagggagtcacgatgagaatgtgagtacttgtgacacaatgccaaaccactctgaaaccatcagtcacactgtgaacgtgcccctgcagacgaccctggggaccttggaggagattgcctgc

SEQ ID NO: 17 - DNA, 1689 bp, Homo sapiens
atgagccggattggcggagccgggctgcccgcagccgcgctcaccggcccgggacgcttccgcatggcccgcgaggagccggcgcccgcggcgttggcggctgccgggcagcccgggggcggcagaggcggcgagctggcgctgcaggggccaggggtcgcccgcagggggcggccatcgctgagccgcgctaaactgcacgggctgcggcacatgtgtgccgggcgcacggcggctgggggctccttccagcggcgggcgctgtgggtgctggccttctgtacatccttcggcttgctgctgtcctggtcctcgaaccgcttgctctactggctcagcttcccgtcacacacgcgggtgcaccgcgagtggagccgccagttacccttccccgccgtcaccgtgtgcaacaacaacccgctgcgcttcccgcgcctctccaagggggacctctactatgccggccactggctcgggctgctgctgcccaaccgcaccgcgcgtccgcttgtcagcgagctgctgcggggcgacgagccgcgccgccagtggttccgcaagctggcggacttccgcctcttcctgcctccgcgccacttcgagggaatcagcgccgccttcatggaccgcctgggccaccagctggaggacatgctgctctcctgcaagtaccgcggcgagctctgcgggccgcacaacttctcctccgtgtttacaaaatatgggaagtgttacatgtttaactcaggcgaggatggcaaacctctgctcaccacggtcaagggggggacaggcaacgggctggagatcatgctggacattcagcaggatgagtacctgcccatctggggagagacagaggaaacgacatttgaagcaggagtgaaagttcagatccacagtcagtctgagccacctttcatccaagagctgggctttggggtggctccagggttccagacctttgtggccacacaggagcagaggctcacatacctgcccccaccgtggggtgagtgccgatcctcagagatgggcctcgacttttttcctgtttacagcatcaccgcctgtaggattgactgtgagacccgctacattgtggaaaactgcaactgccgcatggttcacatgccaggggatgcccctttttgtacccctgagcagcacaaggagtgtgcagagcctgccctaggtctgttggcggaaaaggacagcaattactgtctctgcaggacaccctgcaacctaacccgctacaacaaagagctctccatggtgaagatccccagcaagacatcagccaagtaccttgagaagaaatttaacaaatcagaaaaatatatctcagagaacatccttgttctggatatattttttgaagctctcaattatgagacaattgaacagaagaaggcgtatgaagttgctgccttacttggtgatattggtggtcagatgggattgttcattggtgctagtatccttacaatactagagctctttgattatatttatgagctgatcaaagagaagctattagacctgcttggcaaagaggaggacgaagggagccacgatgagaatgtgagtacttgtgacacaatgccaaaccactctgaaaccatcagtcacactgtgaacgtgcccctgcagacgaccctggggaccttggaggagattgcctgc

SEQ ID NO: 18 - protein, 564 aa, Homo sapiens
MSRIGGAGLPAAALTGPGRFRMAREEPGARGVGRPAGQPGGGRGGERALQGPGVARRGRPSLSRAKLHGLRHMCAGRTAAGGSFQRRALWVLAFCTSFGLLLSWSSNRLLYWLSFPSHTRVHREWSRQLPFPAVTVCNNNPLRFPRLSKGDLYYAGHWLGLLLPNRTARPLVSELLRGDEPRRQWFRKLADFRLFLPPRHFEGISAAFMDRLGHQLEDMLLSCKYRGELCGPHNFSSVFTKYGKCYMFNSGEDGKPLLTTVKGGTGNGLEIMLDIQQDEYLPIWGETEETTFEAGVKVQIHSQSEPPFIQELGFGVAPGFQTFVATQEQRLTYLPPPWGECRSSEMGLDFFPVYSITACRIDCETRYIVENCNCRMVHMPGDAPFCTPEQHKECAEPALGLLAEKDSNYCLCRTPCNLTRYNKELSMVKIPSKTSAKYLEKKFNKSEKYISENILVLDIFFEALNYETIEQKKAYEVAALLGDMGGQMGLFIGASILTILELFDYIYELIKEKLLDLLGKEEDEGSHDENVSTCDTMPNHSETISHTVNVPLQTTLGTLEEIAC

SEQ ID NO: 19 - DNA, 1706 bp, Homo sapiens
ggaatgagccggattggcggagccgggctgcccgcagccgcgctcaccggcccgggacgcttccgcatggcccgagaggagcctggcgcccgcggcgttggccggcctgccgggcagcccgggggcggcagaggcggcgagcgggcgctgcaggggccaggggtcgcccgcagggggcggccatcgctgagccgcgctaaactgcacgggctgcggcacatgtgtgccgggcgcacggcggctgggggctccttccagcggcgggcgctgtgggtgctggccttctgtacatccttcggcttgctgctgtcctggtcctcgaaccgcttgctctactggctcagcttcccgtcacacacgcgggtgcaccgcgagtggagccgccagttacccttccccgccgtcactgtgtgcaacaacaacccgctgcgcttcccgcgcctctccaagggggacctctactatgccggccactggctcgggctgctgctgcccaaccgcaccgcgcgcccgcttgtcagcgagctgctgcggggcgacgagccgcgccgccagtggttccgcaagctggcggacttccgcctcttcctgcctccgcgccacttcgagggaatcagcgccgccttcatggaccgcctgggccaccagctggaggacatgctgctctcctgcaagtaccgcggcgagctctgcgggccgcacaacttctcctccgtgtttacaaaatatgggaagtgttacatgtttaactcaggcgaggatggcaaacctctgctcaccacggtcaagggggggacaggcaacgggctggagatcatgctggacattcagcaggatgagtacctgcccatctggggagagacagaggaaacgacatttgaagcaggagtgaaagttcagatccacagtcagtctgagccacctttcatccaagagctgggctttggggtggctccagggttccagacctttgtggccacacaggagcagaggctcacatacctgcccccaccgtggggtgagtgccgatcctcagagatgggcctcgacttttttcctgtttacagcatcaccgcctgtaggattgactgtgagacccgctacattgtggaaaactgcaactgccgcatggttcacatgccaggggatgcccctttttgtacccctgagcagcacaaggagtgtgcagagcctgccctaggtctgttggcggaaaaggacagcaattactgtctctgcaggacaccctgcaacctaacccgctacaacaaagagctctccatggtgaagatccccagcaagacatcagccaagtaccttgagaagaaatttaacaaatcagaaaaatatatctcagagaacatccttgttctggatatattttttgaagctctcaattatgagacaattgaacagaagaaggcgtatgaagttgctgccttacttggtgatatgggtggtcagatgggattgttcattggtgctagtatccttacaatactagagctctttgattatatttatgagctgatcaaagagaagctattagacctgcttggcaaagaggaggacgaagggagccacgatgagaatgtgagtacttgtgacacaatgccaaaccactctgaaaccatcagtcacactgtgaacgtgcccctgcagacgaccctggggaccttggaggagattgcctgctgacacccctc

(19) United States
(12) Patent Application Publication
(10) Pub. No.: US 2004/0146973 A1
(43) Pub. Date: Jul. 29, 2004

(54) Human acid-sensing ion channel 2b (hASIC2b)

(75) Inventors: Anja Kammesheidt, Laguna Beach, CA (US); Shiazah Z. Malik, Santa Ana, CA (US)

Correspondence Address: DARBY & DARBY P.C., P. O. BOX 5257, NEW YORK, NY, 10150-5257, US

(73) Assignee: Euro-Celtique S.A., Luxembourg (LU)

(21) Appl. No.: 10/704,332

(22) Filed: Nov. 6, 2003

Related U.S. Application Data
(60) Provisional application No. 60/424,496, filed on Nov. 6, 2002.

(51) Int. Cl. C07K 14/705; C07H 21/04

(52) U.S. Cl. 435/69.1; 435/320.1; 435/325; 530/350; 536/23.5

(57) ABSTRACT

The present invention provides nucleic acids encoding a novel protein subunit of a human proton-gated acid-sensing ion channel, designated hASIC2b. The present invention also provides the amino acid sequence of the novel hASIC2b, genetically engineered host cells expressing hASIC2b nucleic acids and amino acids, as well as methods for identifying modulators of hASIC2b-containing ion channels.

This application claims priority from U.S. Provisional Application Serial No. 60/424,496, filed Nov. 6, 2002, which is hereby incorporated by reference in its entirety.

### FIELD OF THE INVENTION

The present invention relates to the human acid-sensing ion channel (hASIC) and its subunits.

### BACKGROUND OF THE INVENTION

Ion channels are polypeptides with tertiary-quaternary structures forming interior pores embedded in plasma cell membranes. Ion channels control the flow of ionic currents across the cell membrane. Numerous ion channels exist in sensory neurons. The electrical excitability of the nerve membrane depends on the membrane's voltage-sensitive ionic permeability system that allows it to use energy stored in ionic concentration gradients. Electrical activity of the nerve is triggered by a depolarization of the membrane, which opens channels that are highly selective for the particular ion, which is then driven across the channel by an electrochemical gradient. There currently exist ion channel families of structurally and functionally related voltage-gated sodium, potassium and calcium channels.

The acid-sensing ion channel (ASIC) subunits ASIC1, ASIC2, ASIC3 and ASIC4 are members of the amiloride-sensitive sodium channel/degenerin family of ion channels (reviewed in Reeh and Kress, Curr Opin Pharmacol, 2001;1:45-50; Waldmann and Lazdunski, Curr Opin Neurobiol, 1998;1:45-50). Structurally, all of the ASIC channels belong to the degenerin/ENaC channel superfamily, and are composed of two hydrophobic segments, intracellular N and C terminals, and a large extracellular loop (Garcia-Anoveros, J. et al., Annu. Rev. Neurosci., 1994;20, 567-594). The subunits combine to form proton-gated channels that are activated by a drop in extracellular pH and are thought to participate in nociceptive pain that accompanies tissue acidosis and inflammation. Protons evoke a sensation of pain, and a variety of hyperalgesic mediators potentiate the pain-inducing actions of low pH, suggesting that proton-gated channels play a central role in nociception (Steen et al., Pain 1996;66:163-170). Acidosis is observed in inflammatory conditions such as chronic joint inflammation, in tumors and after ischemia, and greatly contributes to pain and hyperalgesia. One subunit, ASIC3, is exclusively expressed in rodent dorsal root ganglion (Waldmann et al., J Biol Chem., 1997;272:20975-78). Rat ASIC2, previously named MDEG2, is a splice variant of ASIC2a, previously named MDEG1, and is expressed in brain, spinal cord, and dorsal root ganglion neurons but not in other peripheral tissues and organs (Lingueglia et al., J Biol Chem, 1997;272(47):29778-83). ASIC heteromers also have been identified in neural tissues in co-localization/co-expression studies, and exhibit different current, activation threshold and ionic permeability characteristics than ASIC homomers.

Rat ASIC2b does not form a pH-activated channel by itself, but acts as a modulatory subunit when associated with other ASIC subunits such as ASIC3. In combination with ASIC3, rat ASIC2b is responsible for a prolonged sustained current phase and also changes the ionic selectivity of the channel. One hypothesis is that ASIC2b may be responsible for mediating the tonic phase of pain perception during tissue acidosis (Coscoy et al., J. Biol. Chem., 1999;274:10129-32; Lingueglia et al., supra). Blockade of the sustained current phase may have applications for the treatment of inflammatory pain.

Despite identification of rat sequences for these ASIC subunits, there remains a need in the art to identify human orthologs.

### SUMMARY OF THE INVENTION

The present invention relates to an isolated human ASIC2b (hASIC2b) subunit. In a specific embodiment, the hASIC2b subunit has an amino acid sequence encoded by a nucleotide sequence comprising a sequence depicted in SEQ ID NOs: 1, 6, 8, 10, 15, 16, or 17 (or degenerate variants thereof).

The invention further relates to an isolated ASIC2b subunit protein having an amino acid sequence with at least 95% and preferably at least 97% or 98%, sequence identity to human ASIC2b protein having an amino acid sequence as depicted in SEQ ID NOs: 7, 9, 11, or a consensus sequence of these as depicted in SEQ ID NO:14, and preferably having the same function as the native hASIC2b subunit.

Also provided by the invention are nucleic acids, e.g., genomic or cDNA clones, encoding the ASIC2b subunit proteins discussed above (i.e., SEQ ID NOs: 7, 9, 11, and 14). In another embodiment, the invention provides an isolated nucleic acid encoding an ASIC2b subunit, which nucleic acid hybridizes under stringent conditions to a nucleic acid having the sequence of any one of SEQ ID NOs: 1, 6, 8, 10, 15, 16, and 17 or complements thereof (and degenerate variants thereof), and which preferably encodes a protein having the same function as the native subunit protein.

The isolated nucleic acids encoding ASIC2b subunits of the invention can be part of vectors, e.g., for cloning, expression, and/or expansion. For example, an expression vector comprises the nucleic acid of the invention encoding the ASIC2b subunit operably associated with an expression control sequence. The invention further provides host cells and non-human transgenic animals containing such an expression vector, and methods for producing an ASIC2b subunit polypeptide using such host cells.

In addition, the invention provides an isolated nucleic acid oligonucleotide, such as a primer or probe, of at least 10 bases, more particularly at least 20 bases, and more particularly at least 30 bases, which oligonucleotide has a sequence identical to a corresponding nucleotide sequence of the same number of contiguous nucleotides in any one of SEQ If) NOs: 1, 6, 8, 10, 15, 16, or 17, or their complement, which nucleotide sequence is different from corresponding oligonucleotide sequences of known ASIC ortholog subunits such as, e.g., the rat ASIC2b. The invention also provides an antibody that preferentially binds an ASIC2b subunit protein of the invention (e,g., to a protein having an amino acid sequence shown in SEQ ID NOs: 7, 9, 11, and 14) compared to known ASIC ortholog subunits.

The present invention further contemplates a method for detecting a human ASIC2b subunit, which method comprises: (i) detecting mRNA encoding the human ASIC2b subunit in a sample from a cell suspected of expressing human ASIC2b subunit; or (ii) detecting the human ASIC2b subunit protein with an antibody of the invention. Also contemplated is a method for identifying a host cell that expresses a hASIC2b polypeptide, which method comprises: (i) contacting a test host cell with an oligonucleotide complementary to an mRNA sequence encoding a hASIC2b polypeptide having the amino acid sequence of SEQ ID NO:14; and (ii) observing whether the oligonucleotide binds to an mRNA sequence transcribed by the test host cell.

The present invention also contemplates an assay system for identifying modulators of human ASIC2b subunit-containing channels. The assay system comprises at least one host cell genetically engineered to express a specific combination of ASIC subunits as a functional acid sensing ion channel, which combination includes at least one human ASIC2b subunit of the invention, and wherein the resulting functional acid sensing ion channel can be used to screen for and thereby identify modulators of pH activated responses. The assay may comprise the steps of: (i) contacting a host cell that has been genetically engineered to express or overexpress a functional acid sensing ion channel (ASIC) comprising at least one hASIC2b subunit protein with a test compound under conditions that would otherwise activate ion channel activity of said functional ASIC; and (ii) comparing the ion channel activity in the presence of the test compound to a control. The control may be, for example, the ion channel activity of the host cell in the absence of test compound and under conditions that would otherwise activate ion channel activity, or the ion channel activity of a second host cell of the same type in the absence of test compound and under conditions that would otherwise activate ion channel activity. In particular embodiments, the host cell has been genetically engineered to express or overexpress a hASIC2b-encoding nucleic acid molecule endogenous to said host cell, or the host cell has been genetically engineered to express or overexpress endogenous nucleic acid molecules encoding the functional acid sensing ion channel comprising the hASIC2b subunit protein. The modulators identified may be either antagonists or agonists of the ion channel activity.

### BRIEF DESCRIPTION OF THE DRAWINGS

FIG. 1 depicts the predicted genomic DNA sequence of hASIC2b (SEQ ID NO:1; upper sequence), as identified from draft chromosome 17 of the human genome, aligned with rat ASIC2b (SEQ ID NO:2; middle sequence), as well as the consensus. “S” indicates a gap in the sequence.

FIG. 2 shows cDNA sequence alignment of hASIC2a (SEQ ID NO:4; first sequence) and the three hASIC2b clones CB12 (SEQ ID NO:6; second sequence), FB4 (SEQ ID NO:8; third sequence) and SC2 (SEQ ID NO:10; fourth sequence), as well as the consensus. Shaded regions indicate differences between hASIC2b clones and hASIC2a. The hASIC2b forward primer starts at positions 97, 97, and 98 for CB12, FB4, and SC2, respectively, with the adenosine (“a”) residue of the start codon for all of CB12, FB4, and SC2 being at position 100 of the sequence alignment. The adenosine residue of the start codon for hASIC2a is at position 275 of the alignment. hASIC2b and hASIC2a share identity from position 854 and forward. The thymidine (t) of the stop codon for all sequences is located at position 1835. The first residue of the hASIC2b reverse primer is located at position 1846 of the alignment.

FIG. 3 shows amino acid sequence alignment of hASIC2a (SEQ ID NO:5; upper sequence) and the three hASIC2b clones CB12 (SEQ ID NO:7; second sequence), FB4 (SEQ ID NO:9; third sequence) and SC2 (SEQ ID NO:11, fourth sequence), as well as the consensus. Shaded regions indicate differences between hASIC2b clones and hASIC2a. From amino acid 1 to amino acid 236, the hASIC2b sequence is unique. From amino acid 237 and forward, hASIC2a and hASIC2b share substantial identity.

FIG. 4 shows cDNA sequence alignment of rat ASIC2b (SEQ ID NO:2; upper sequence) and the three hASIC2b clones CB12 (SEQ ID NO:6; second sequence), FB4 (SEQ ID NO:8; third sequence) and SC2 (SEQ ID NO:10; fourth sequence), as well as the consensus. Shaded regions indicate differences between hASIC2b clones and rat ASIC2b. The first residue of the hASIC2b forward primer is located at positions 13, 13, or 14, for CB12, FB4, and SC2, respectively. The adenosine of the start codon is at position 16 of the alignment. The thymidine residue of the hASIC2b stop codon is at position 1705 of the alignment, and the first residue of the reverse hASIC2b primer is located at position 1715 of the alignment.

FIG. 5 shows amino acid sequence alignment of rat ASIC2b (SEQ ID NO:3; upper sequence) and the three hASIC2b clones CB12 (SEQ ID NO:7; second sequence), FB4 (SEQ ID NO:9; third sequence) and SC2 (SEQ ID NO:11; third sequence), as well as the consensus. Shaded regions indicate differences between hASIC2b clones and rat ASIC2b.

### DETAILED DESCRIPTION OF THE INVENTION

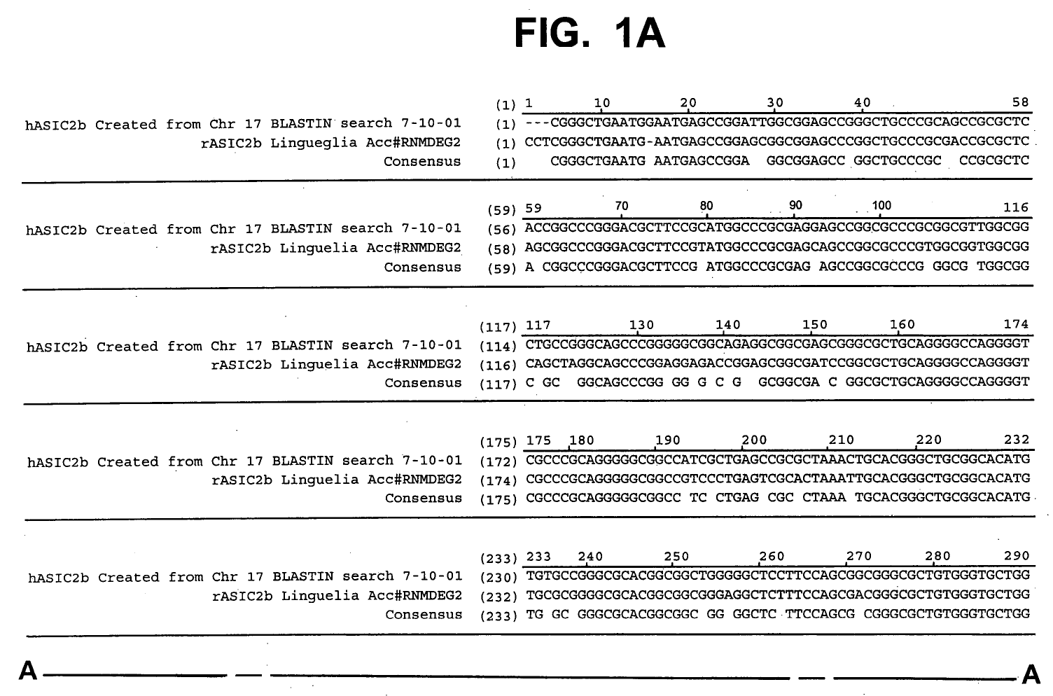

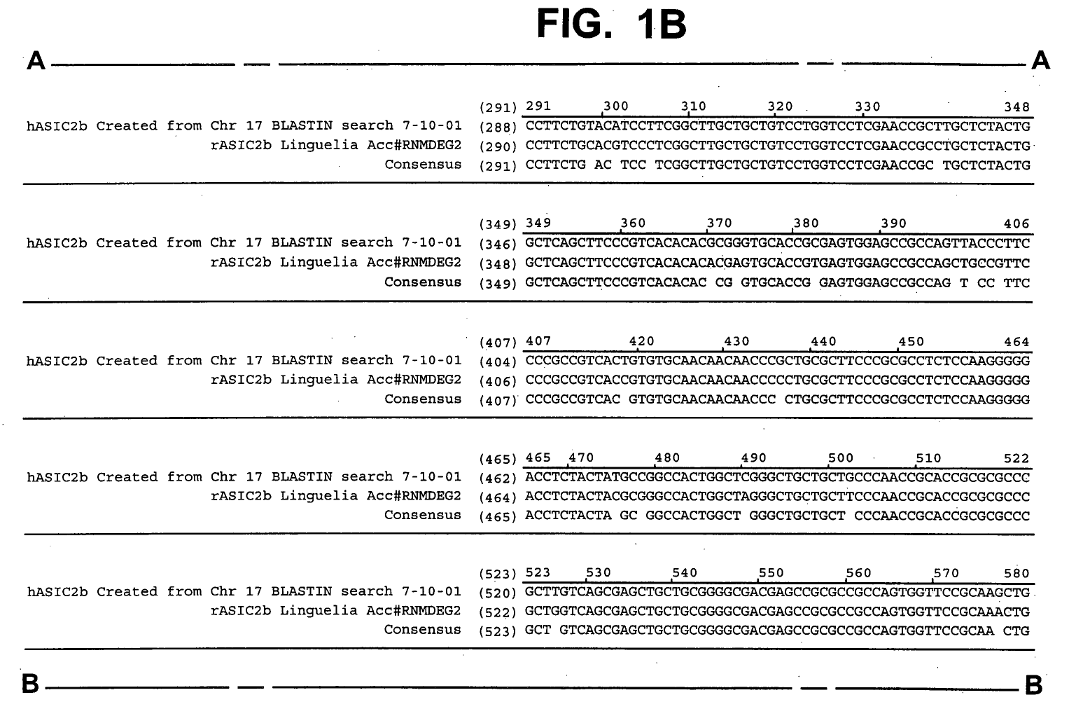

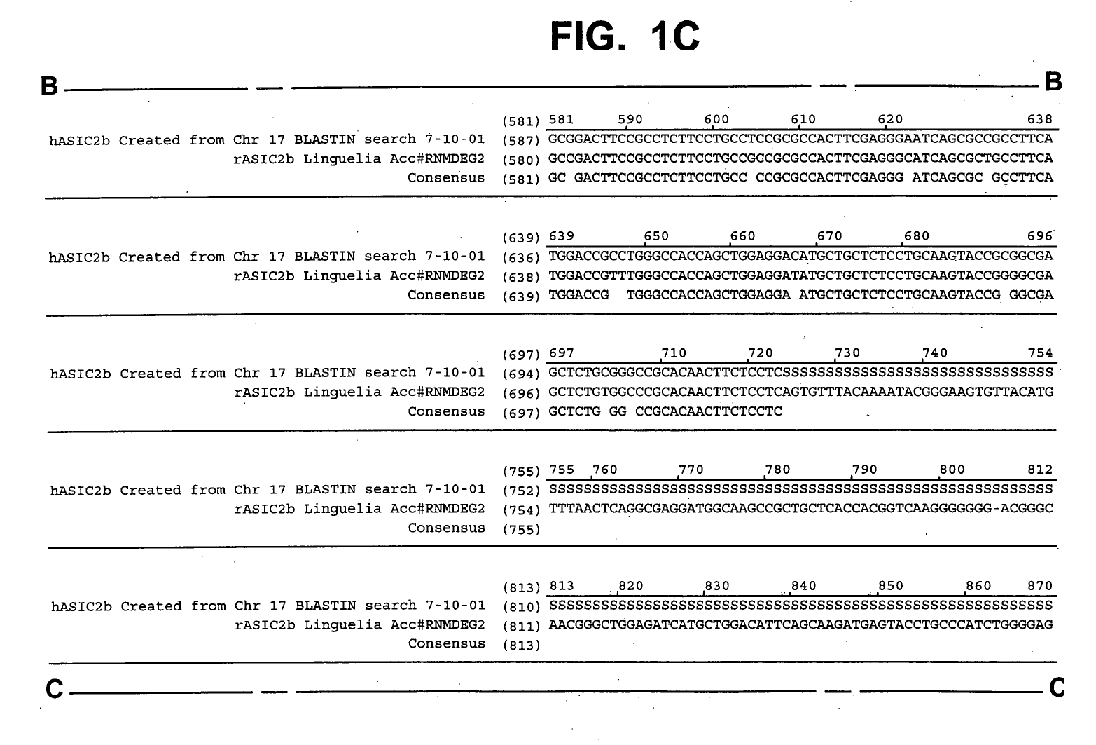

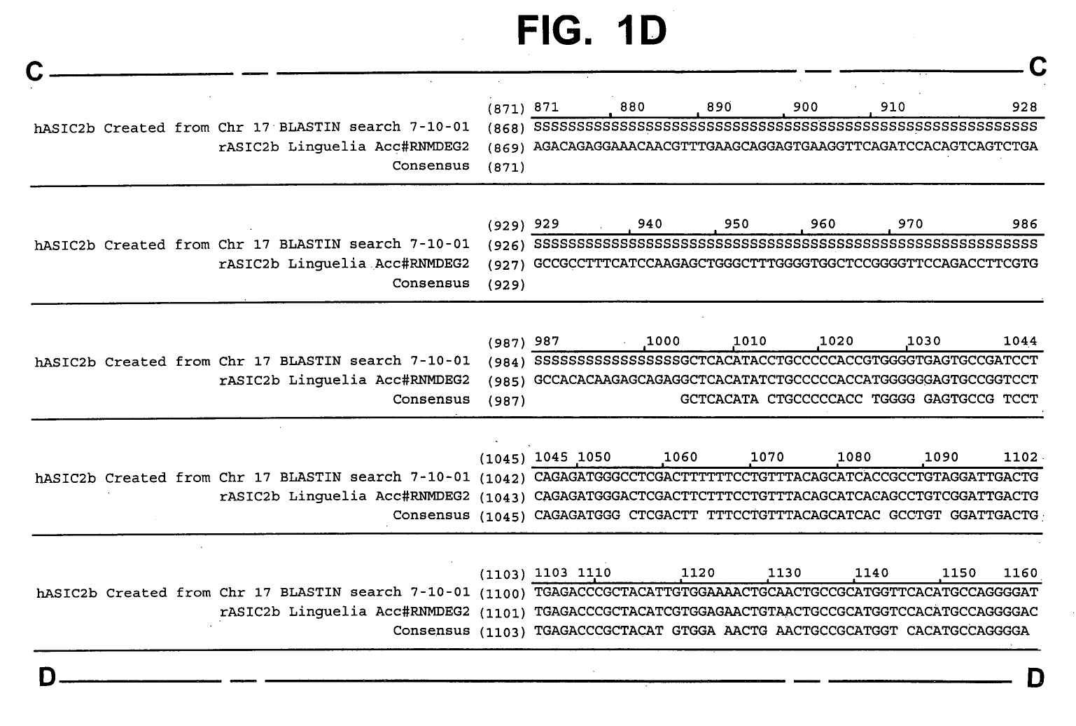

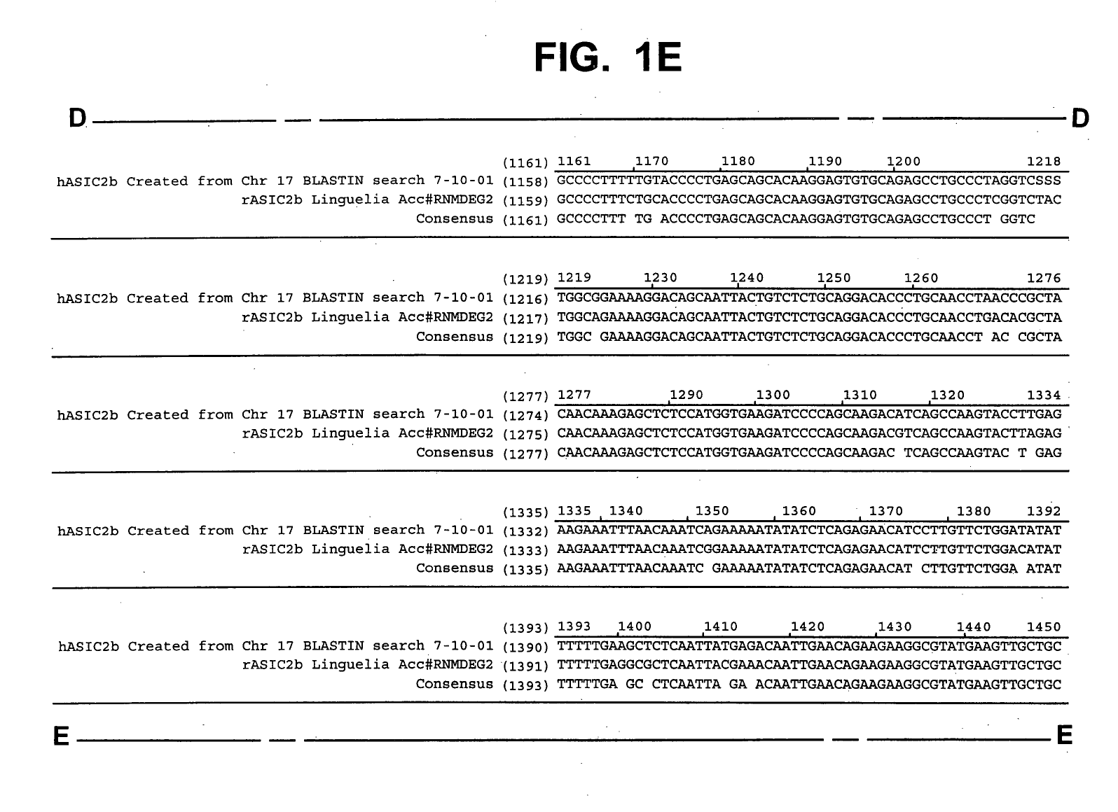

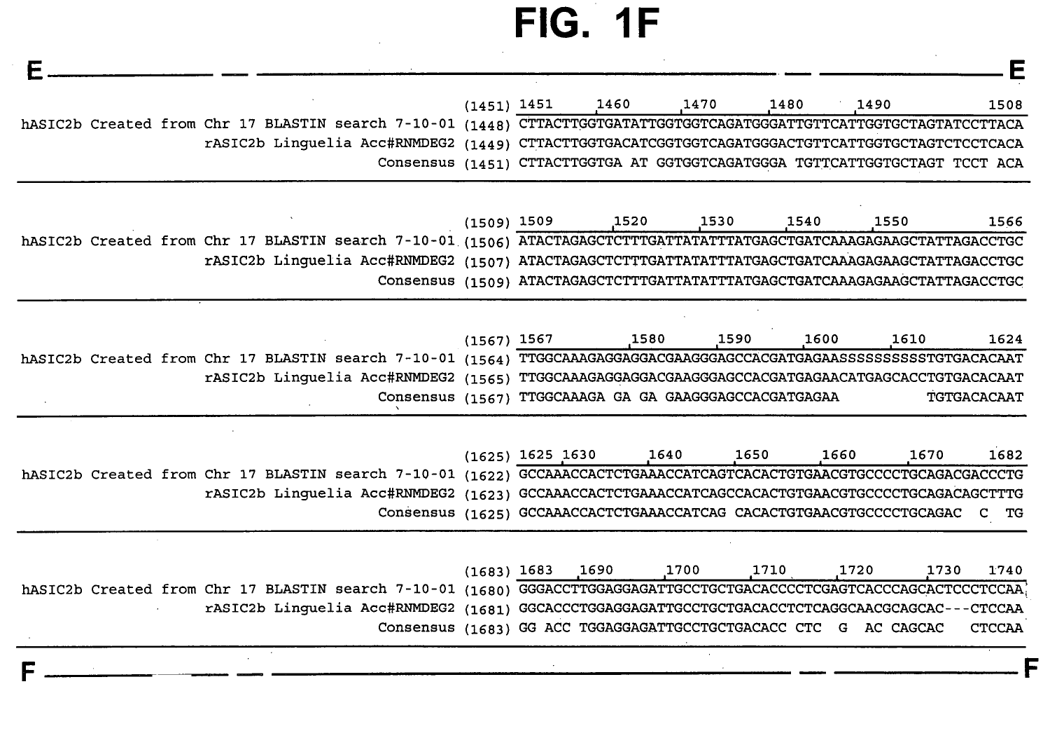

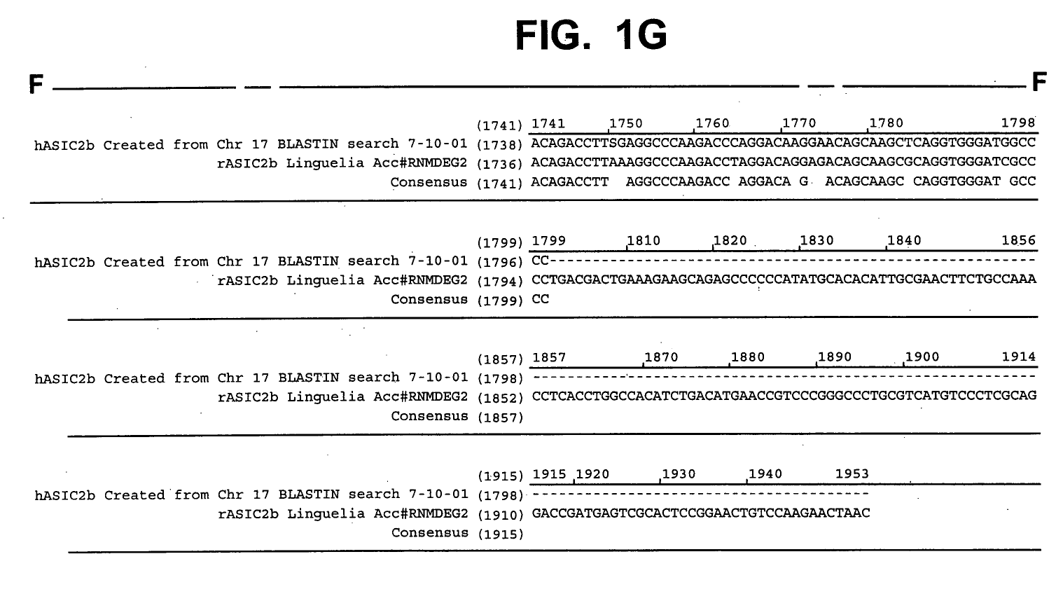

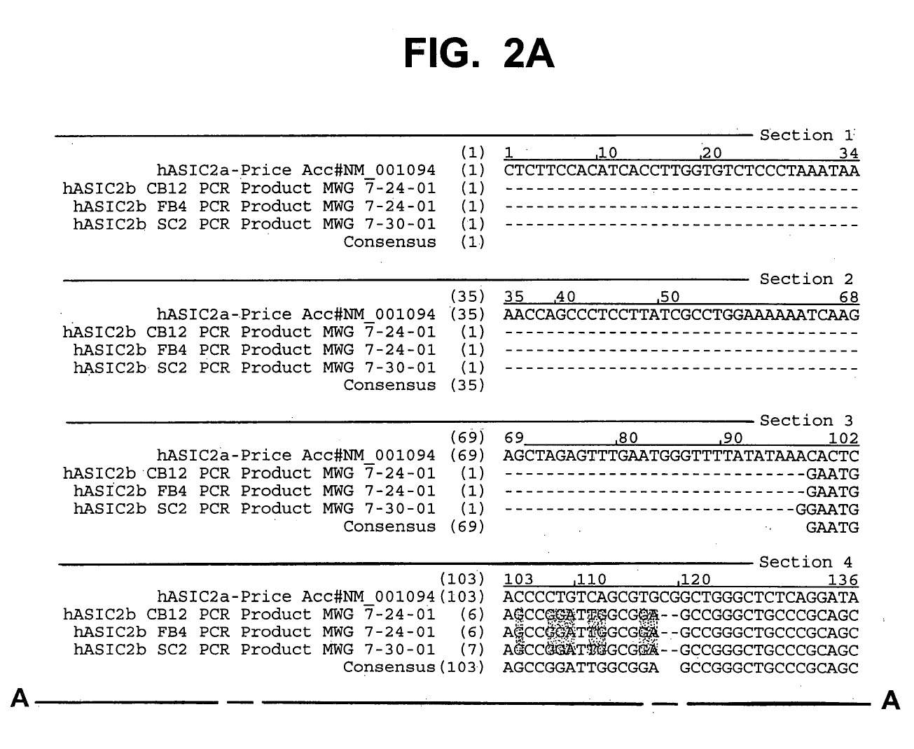

The present invention is based, in part, on the discovery of a human ortholog of the ASIC2b subunit. A human ASIC2b-encoding nucleic acid was cloned from human spinal cord, fetal brain, and adult cerebellum using ASIC2b-specific primers designed from a sequence identified by searching the NCBI Human Genome database, using the rat MDEG2 sequence (described in GenBank accession no. Y14635-SEQ ID NO:2) and the BLASTN and TBLASTN algorithms. A partial sequence corresponding to the rat sequence was identified on chromosome 17 of the working draft sequence of the human genome (described in GenBank accession no. NT_{—}010679.3|Hs17_{—}10836). Coding regions of human ASIC2b, including the likely start and stop codons were identified by comparison to the rat ASIC2b sequence, but contained several gaps. See FIG. 1. A full length genomic human ASIC2b (hASIC2b) of 1702 bp, having an open reading frame of 1689 bp (1692 including the stop codon), was amplified by reverse-transcriptase PCR using primers corresponding to the presumed start and stop sites. PCR fragments were cloned into the mammalian expression vector pEF6/V5 HIS TOPO and the complete DNA sequence was determined (SEQ ID NO:1).

Protein expression was confirmed by in vitro transcription/translation of the three clones from human adult cerebellum, fetal brain, and adult spinal cord, respectively (SEQ ID NOs: 7, 9 and 11), and a protein of approximately 63 kD protein, corresponding to the expected, calculated molecular weight based on the predicted protein sequence, was identified.

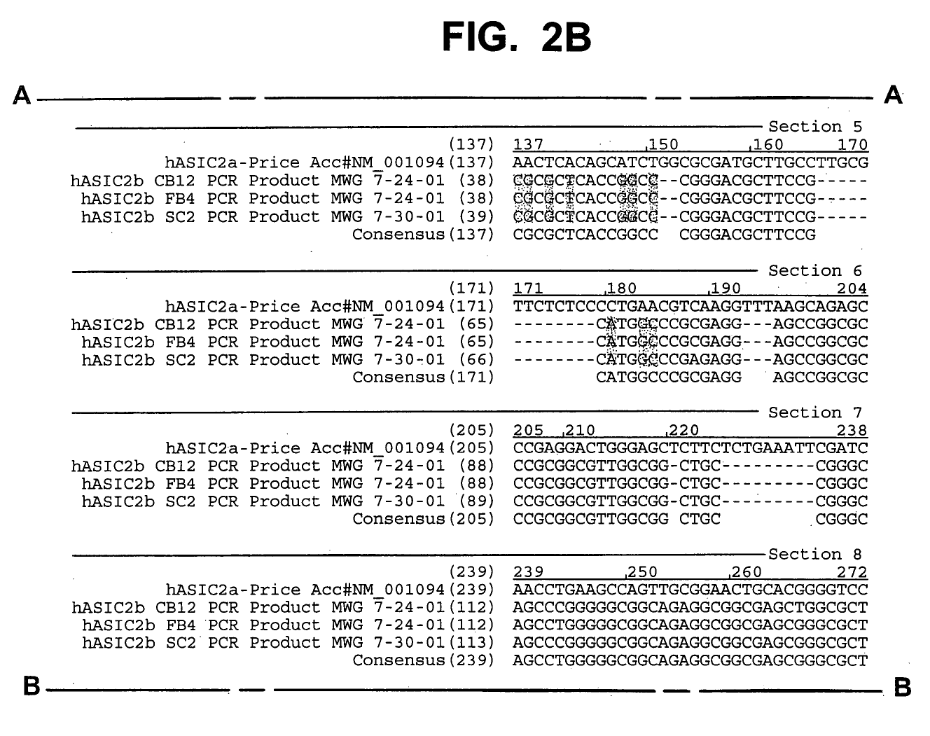

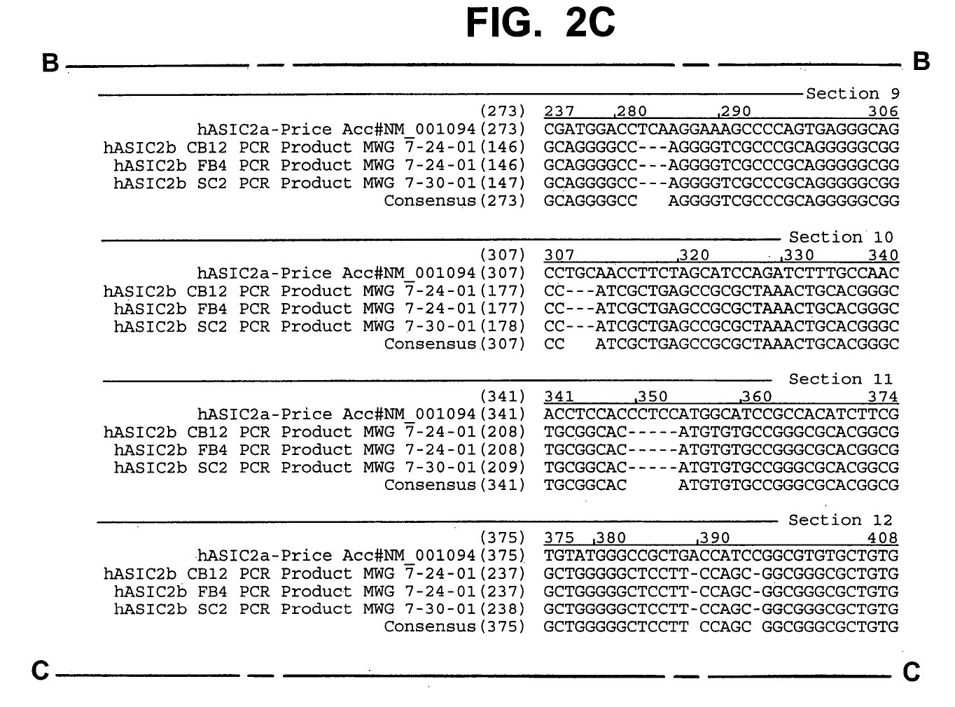

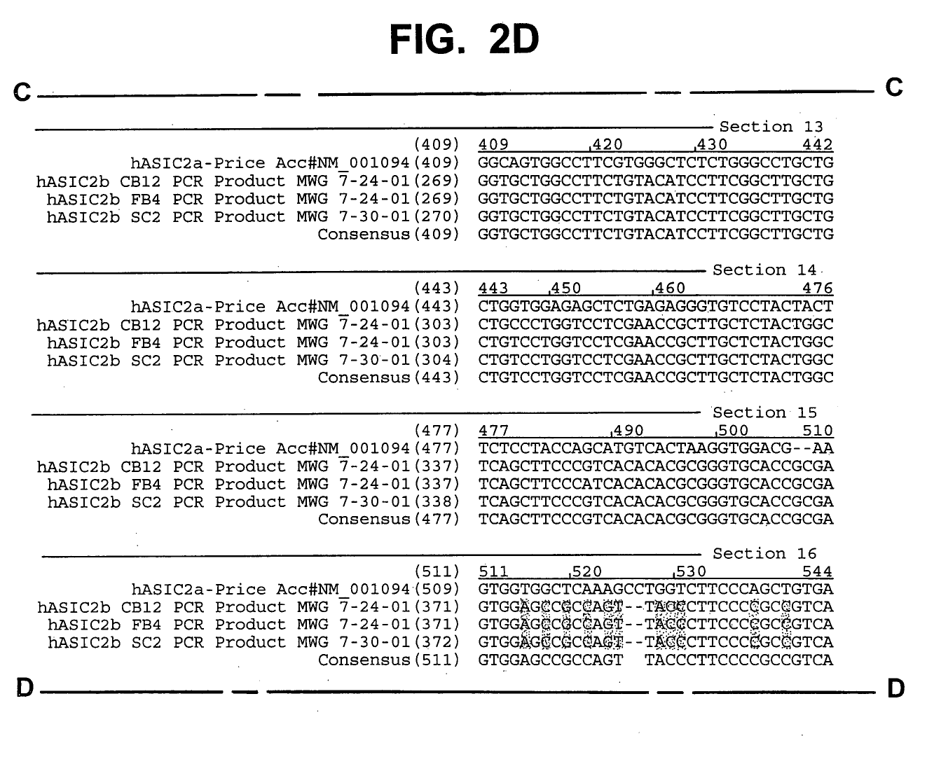

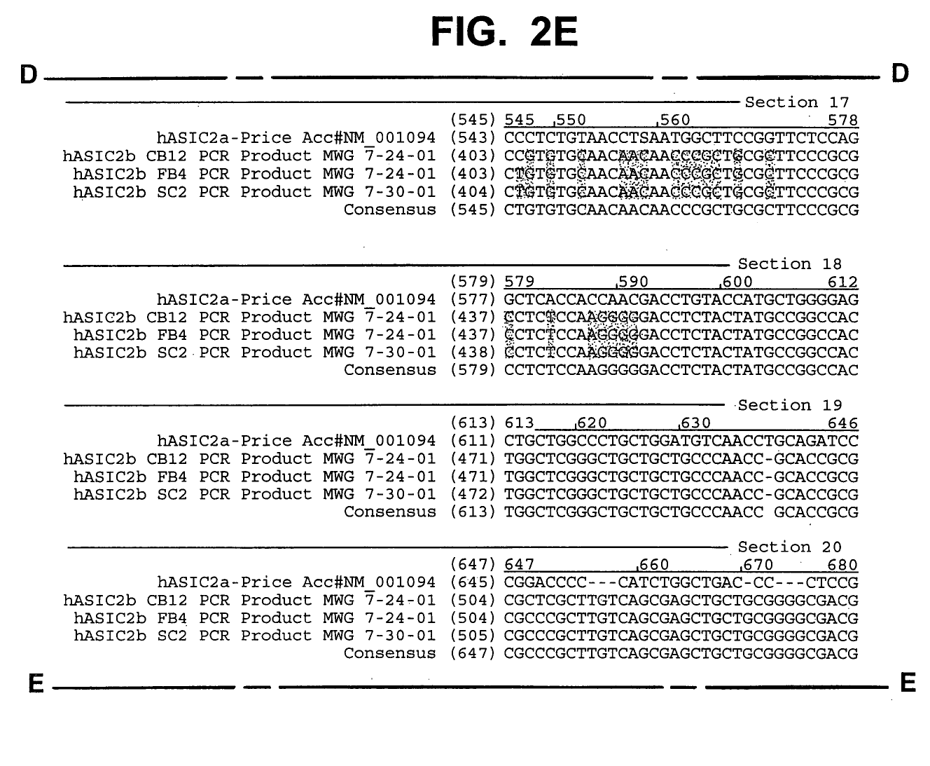

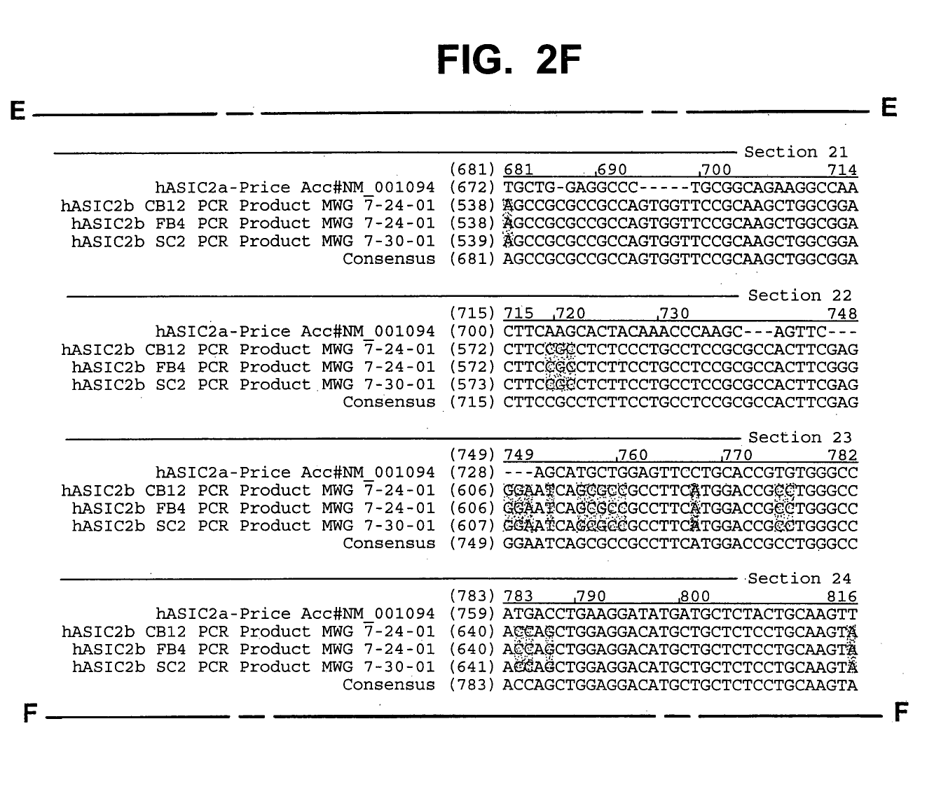

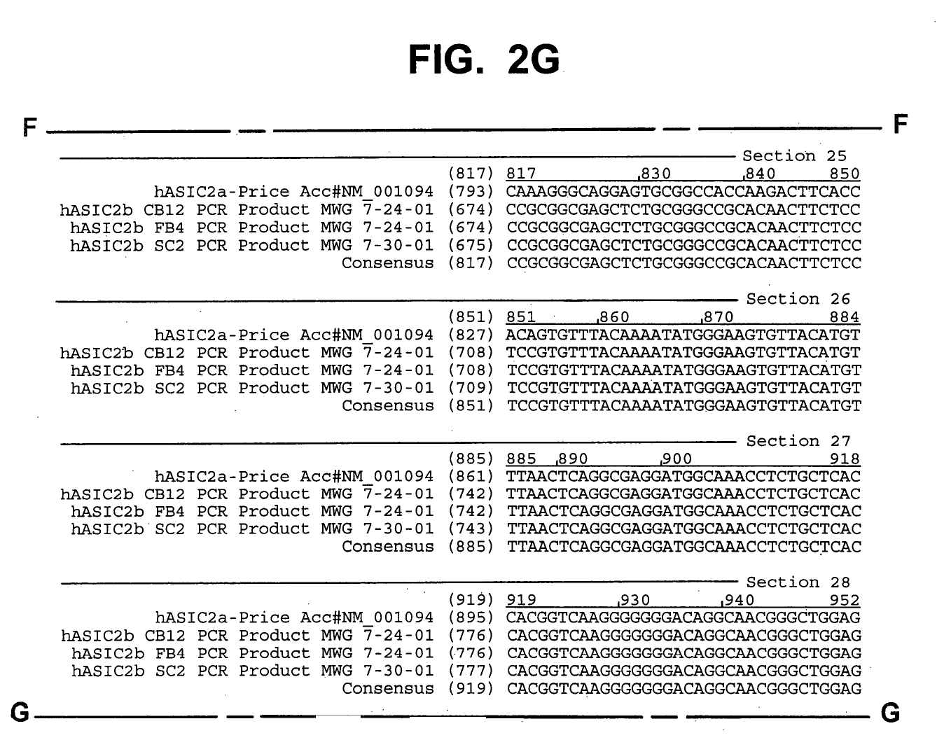

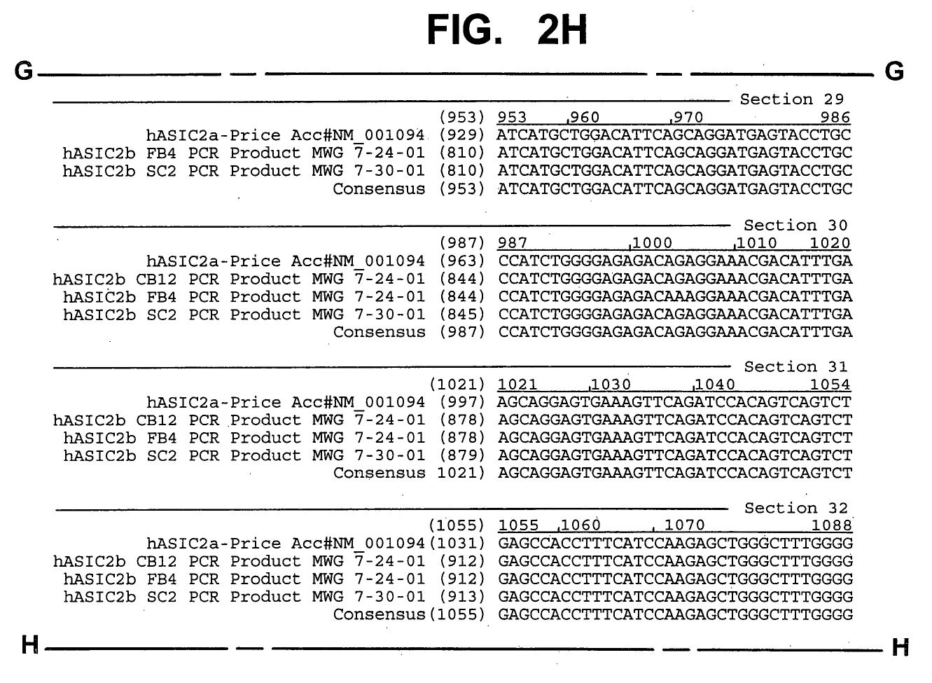

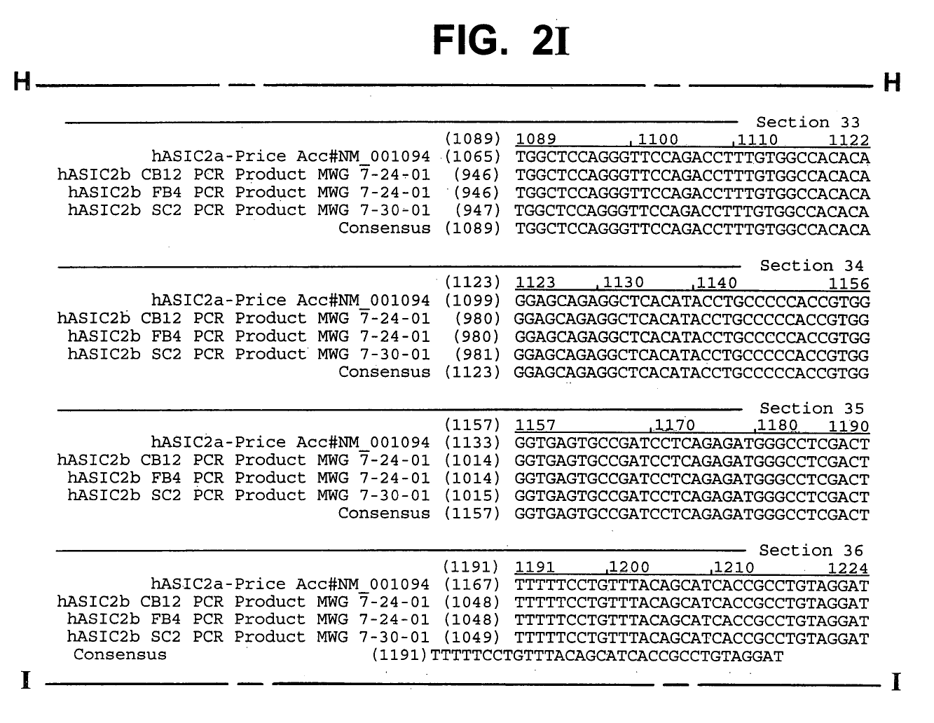

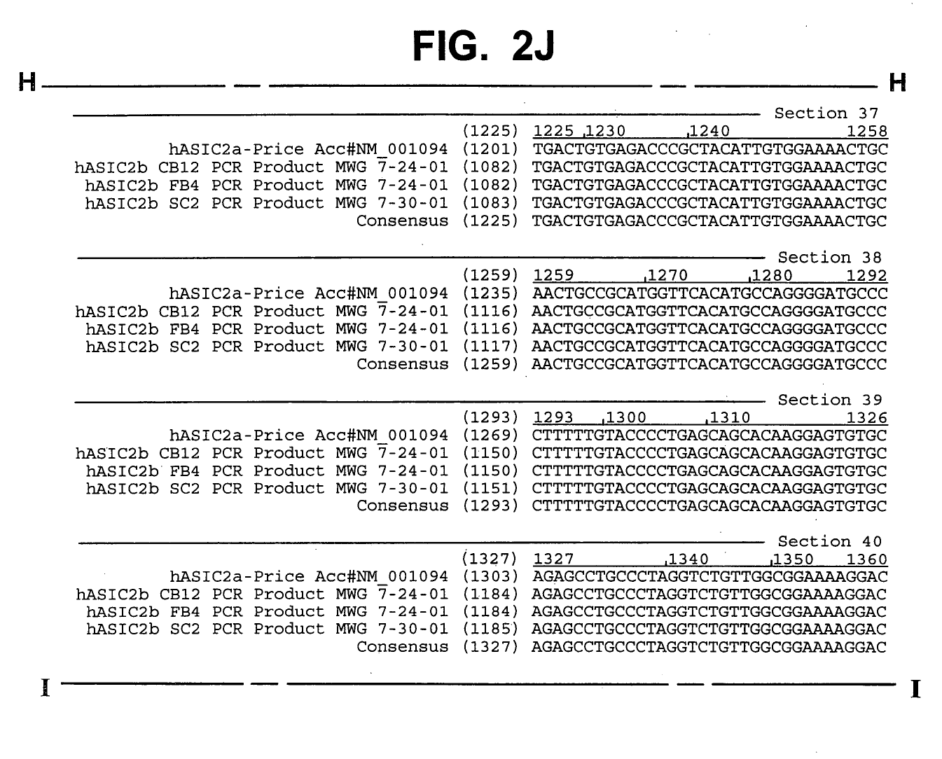

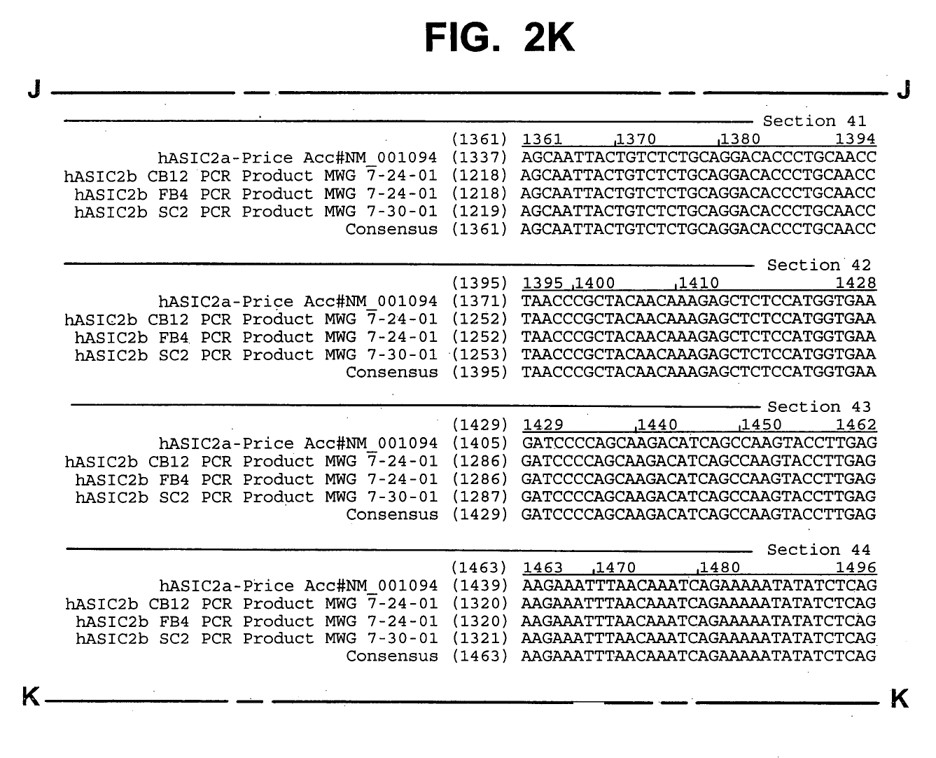

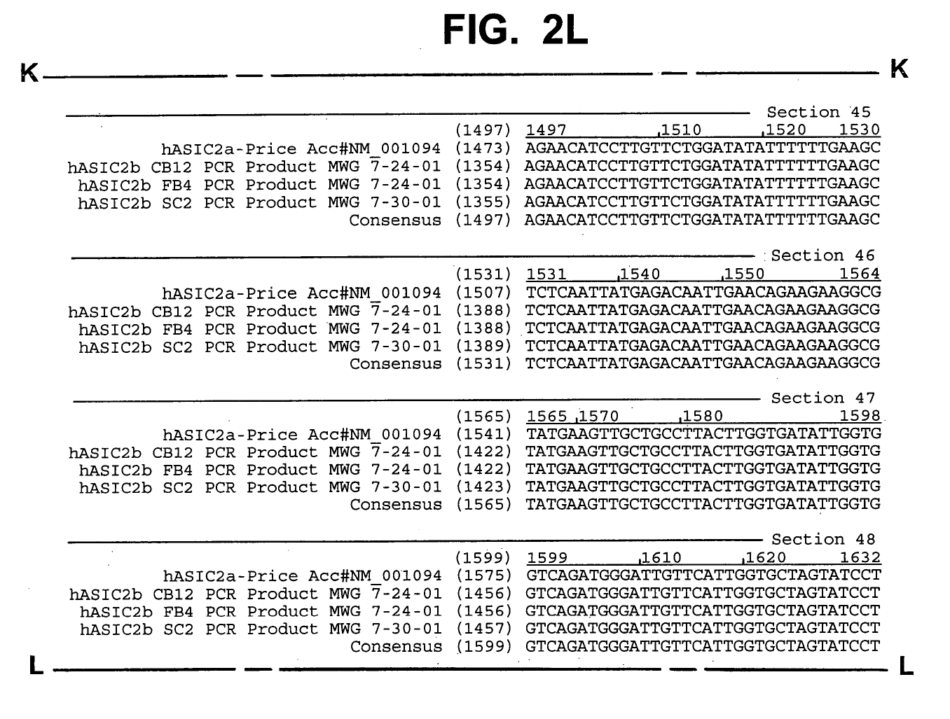

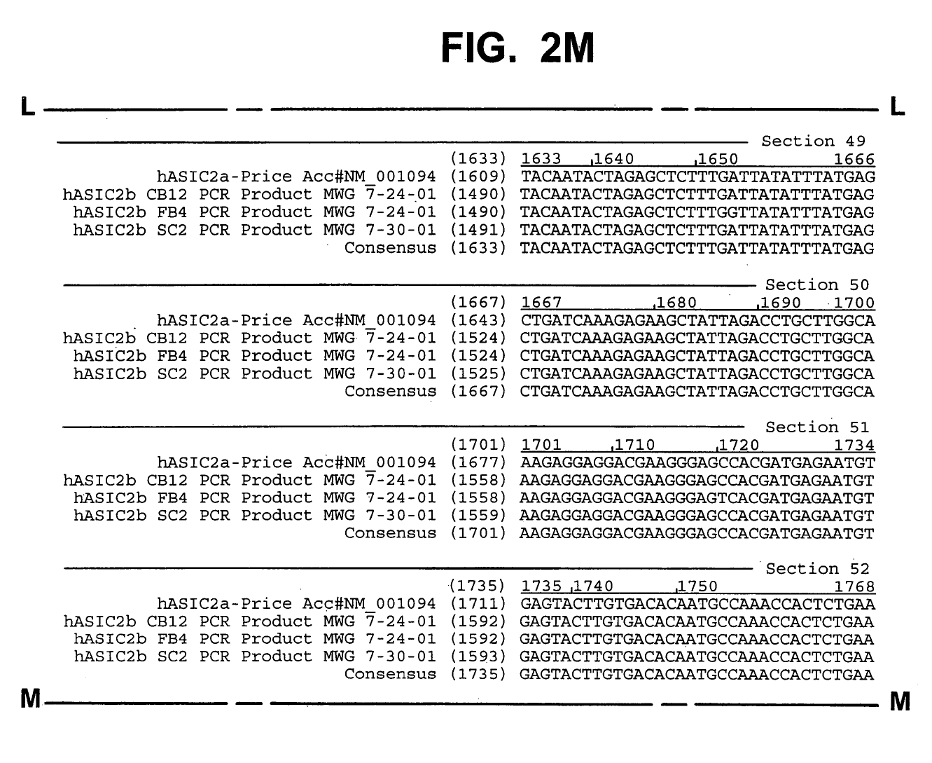

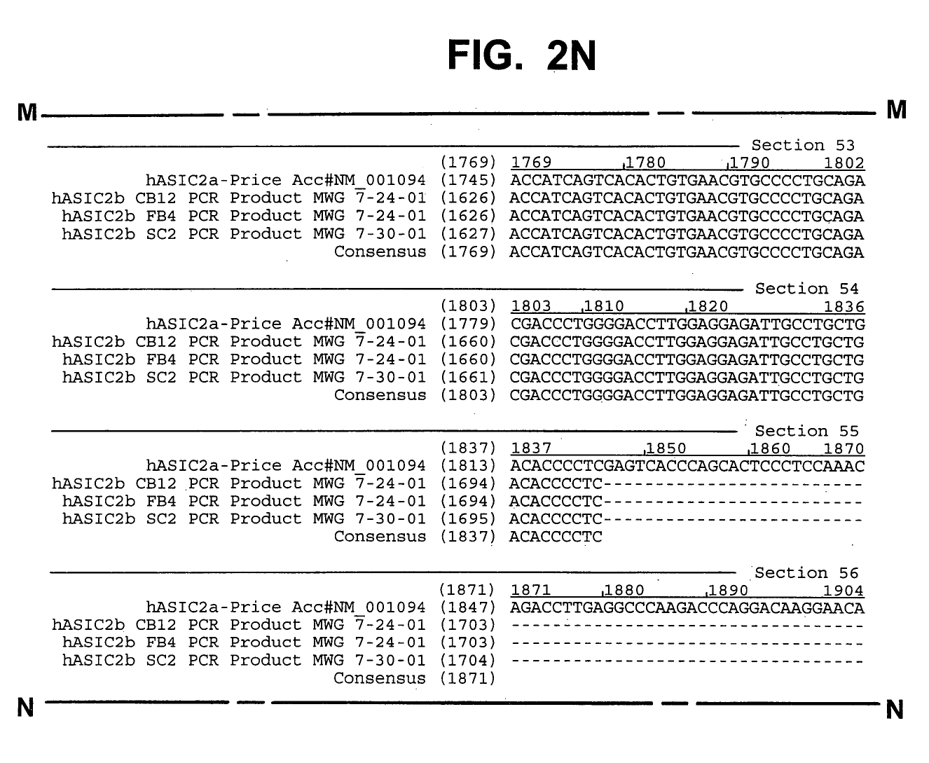

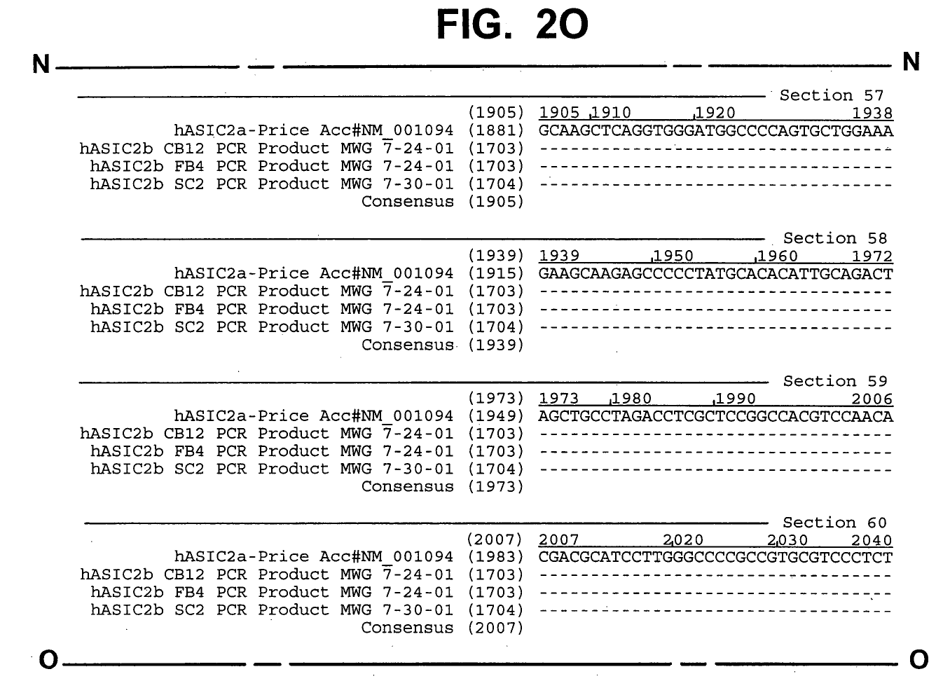

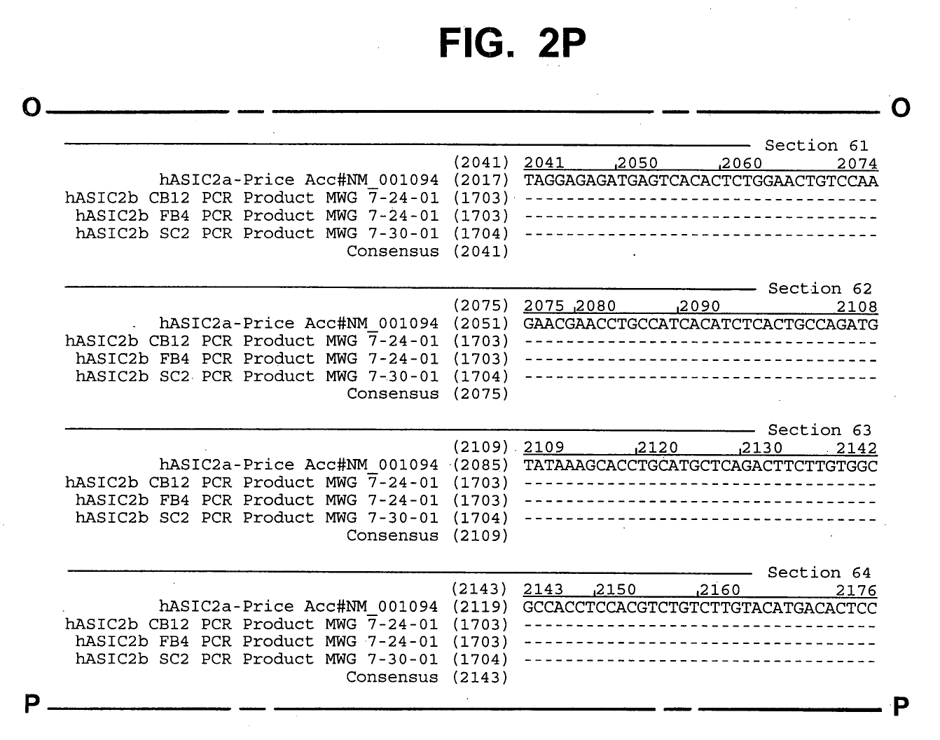

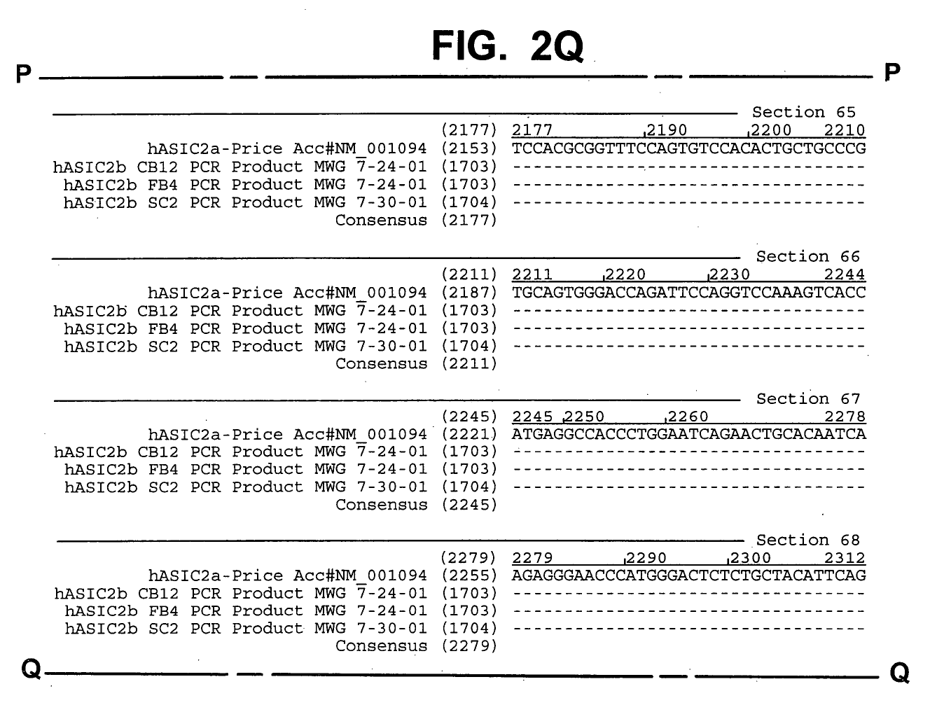

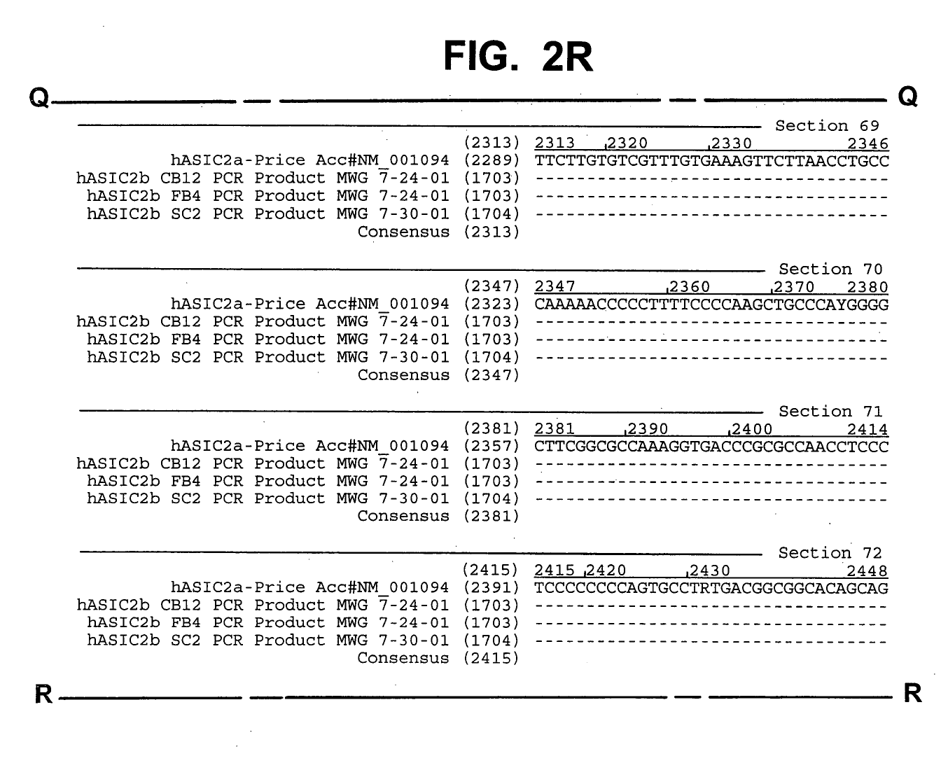

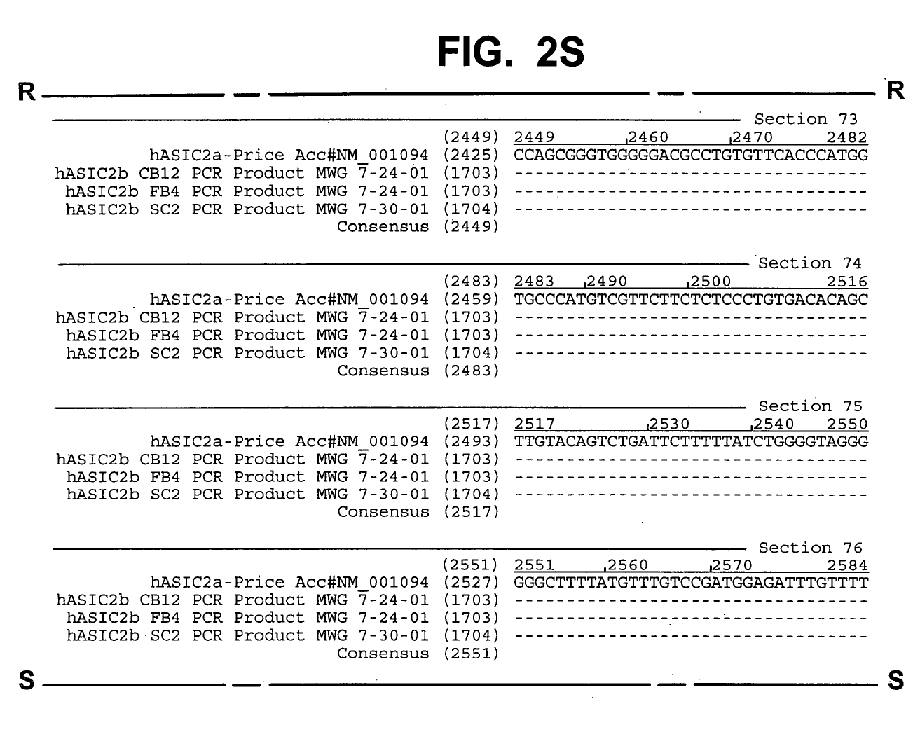

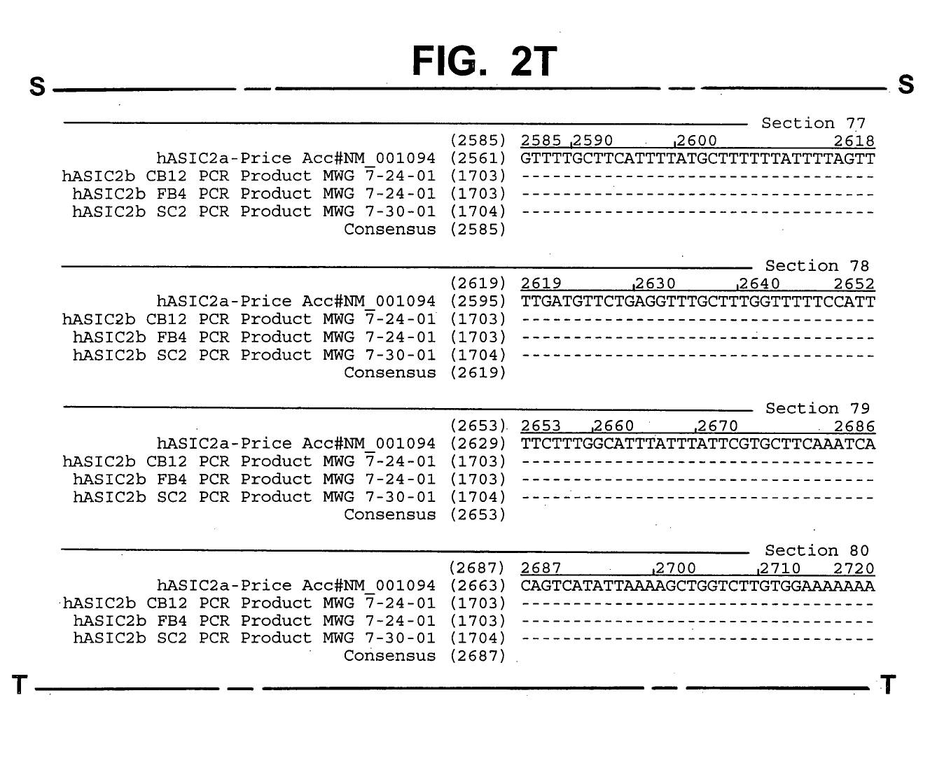

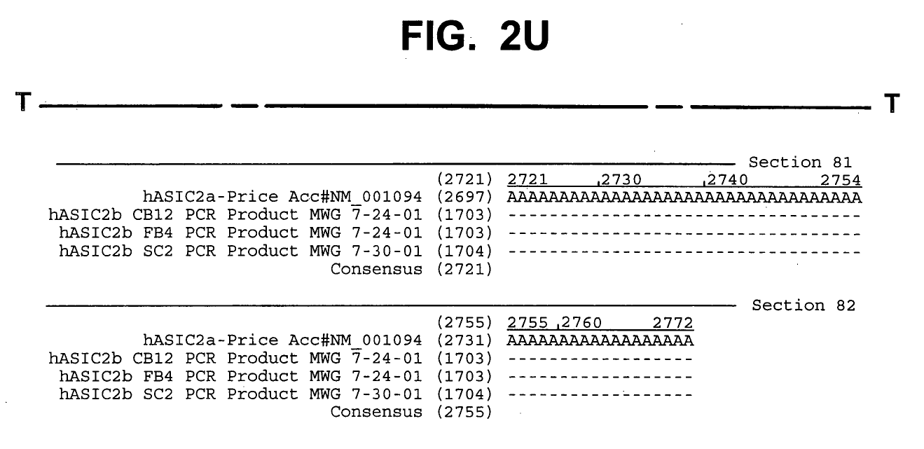

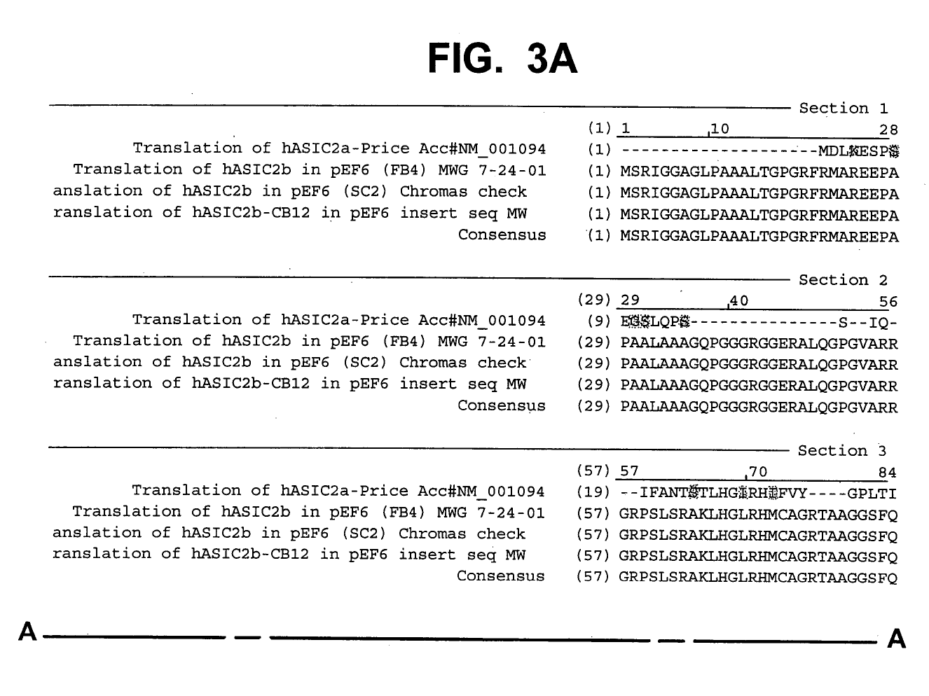

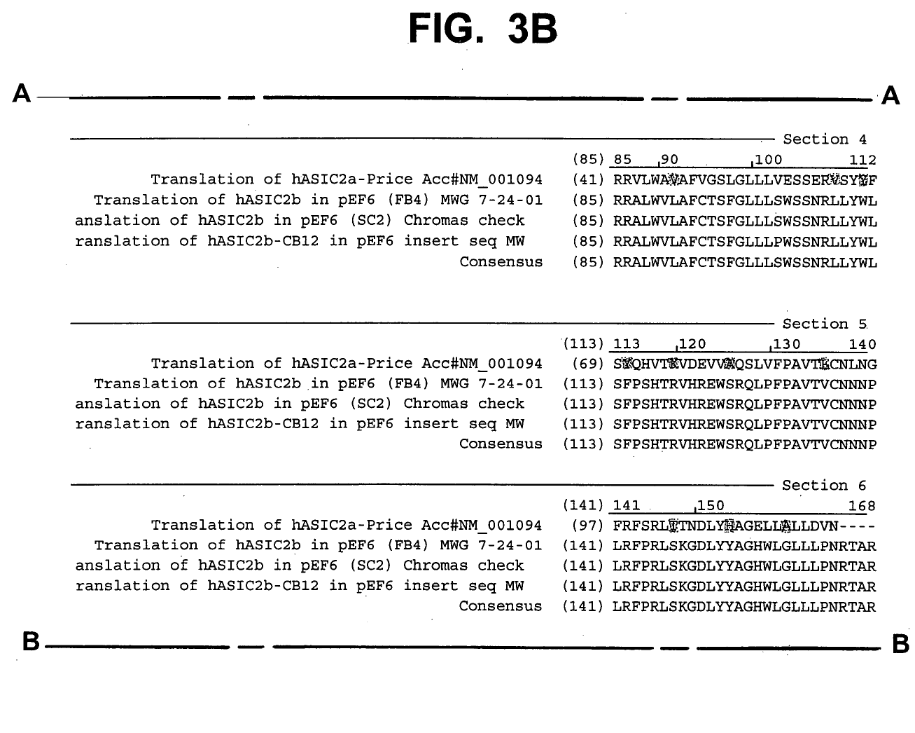

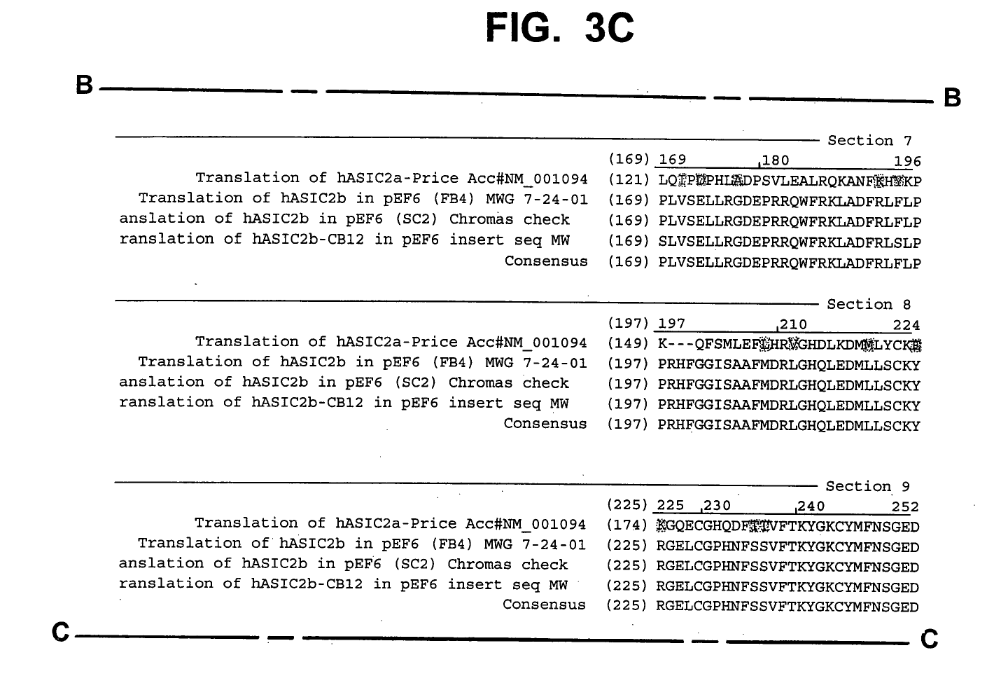

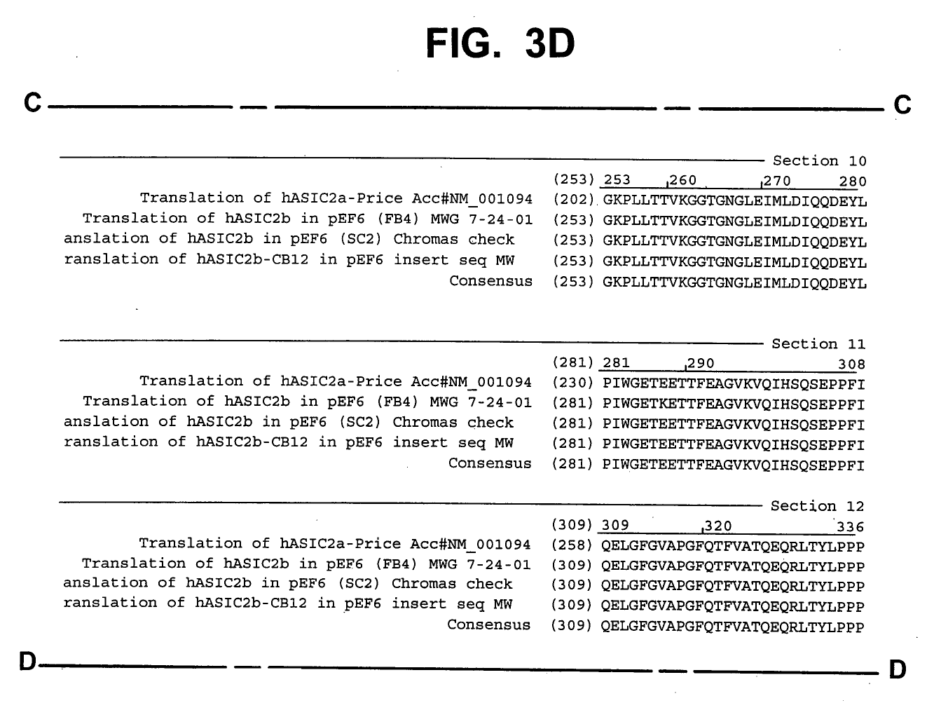

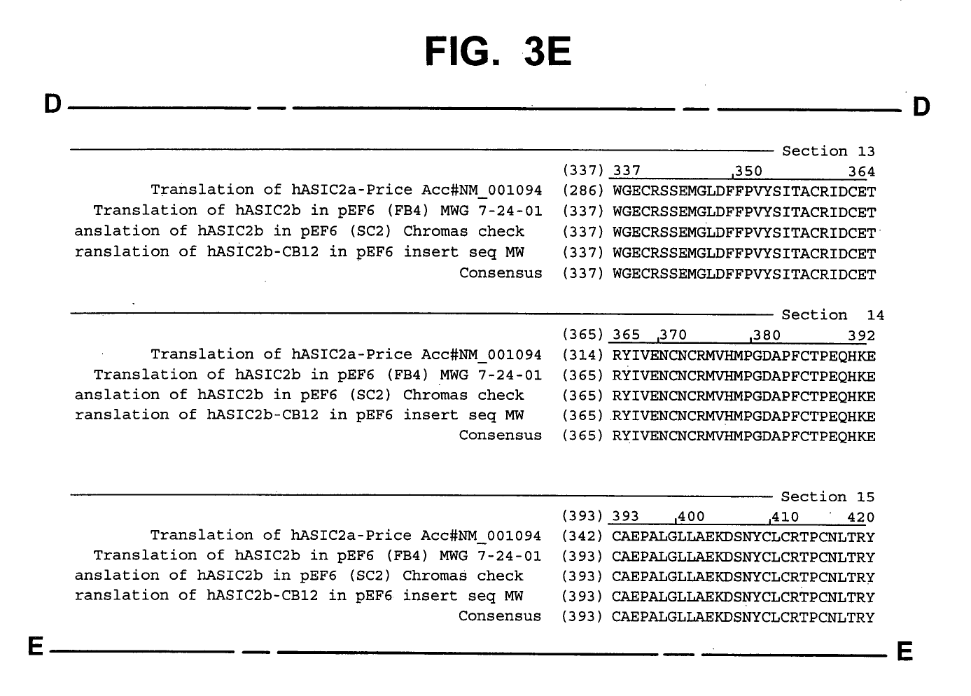

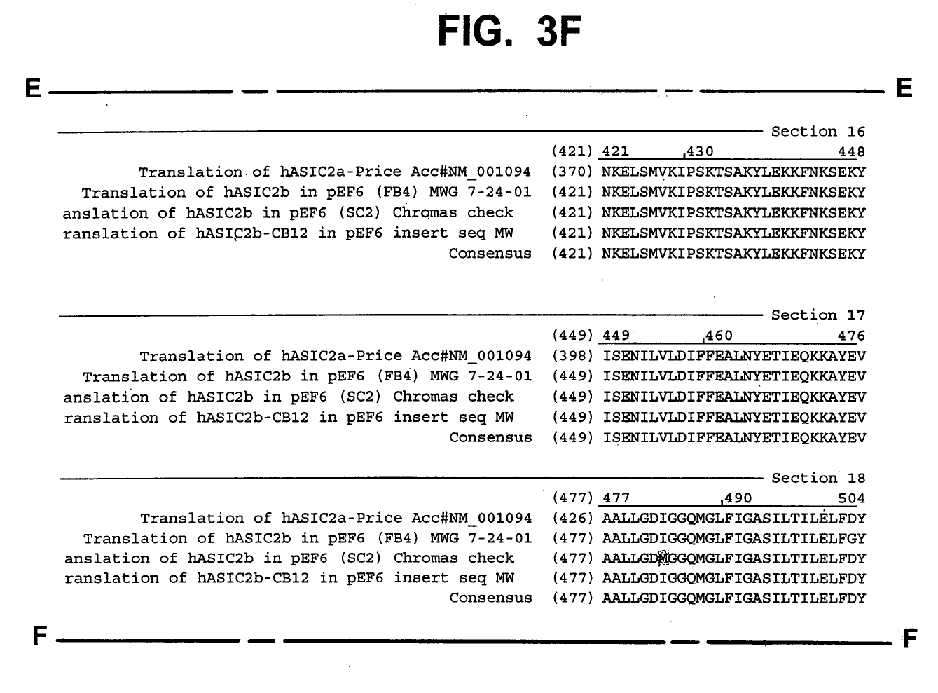

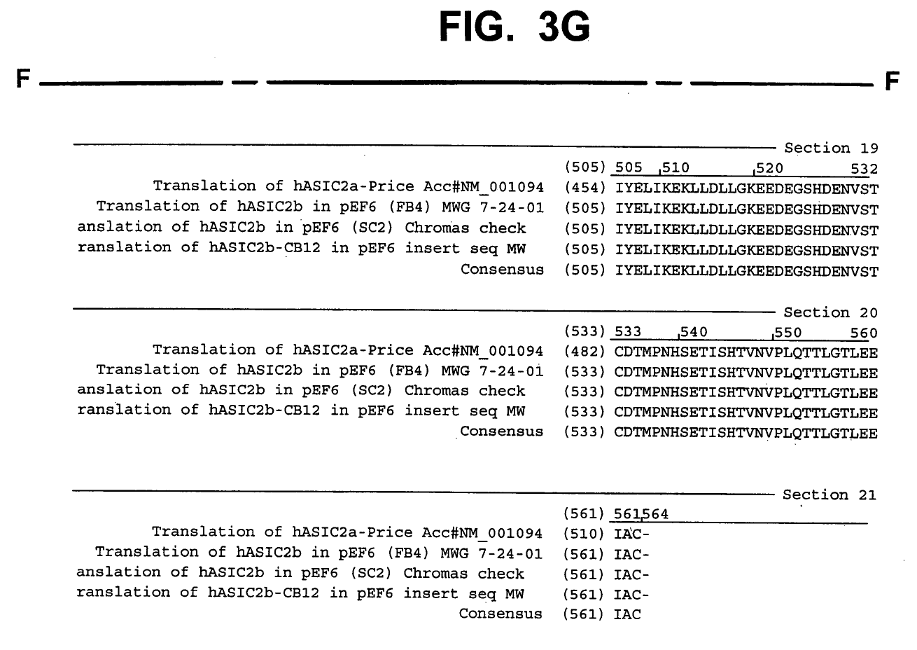

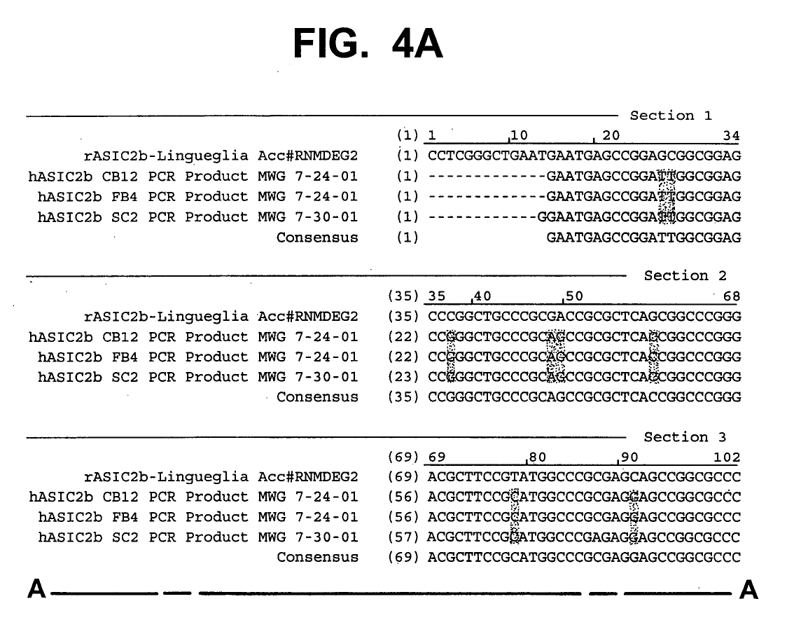

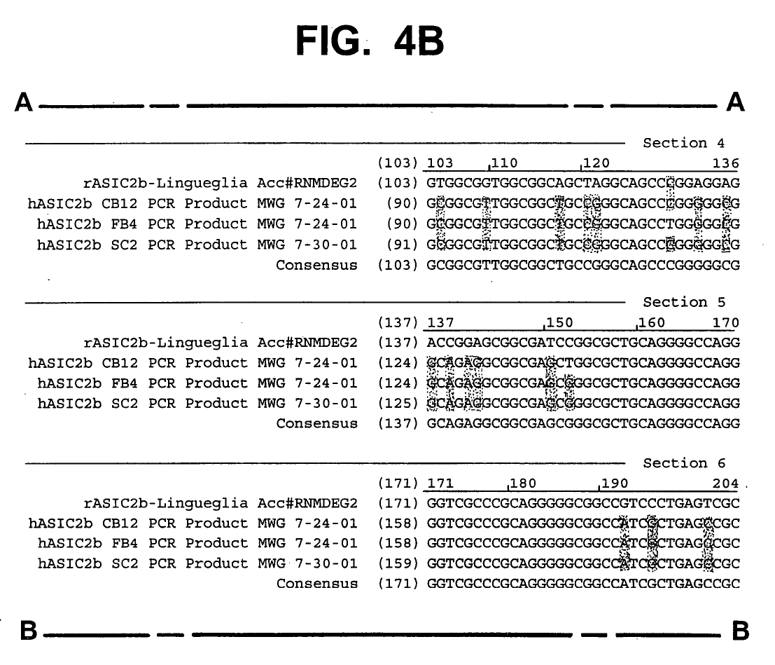

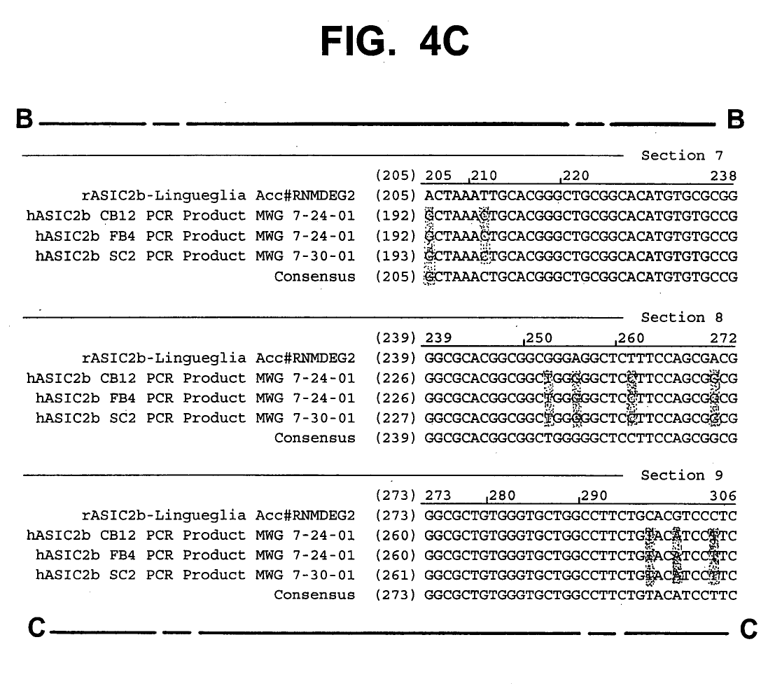

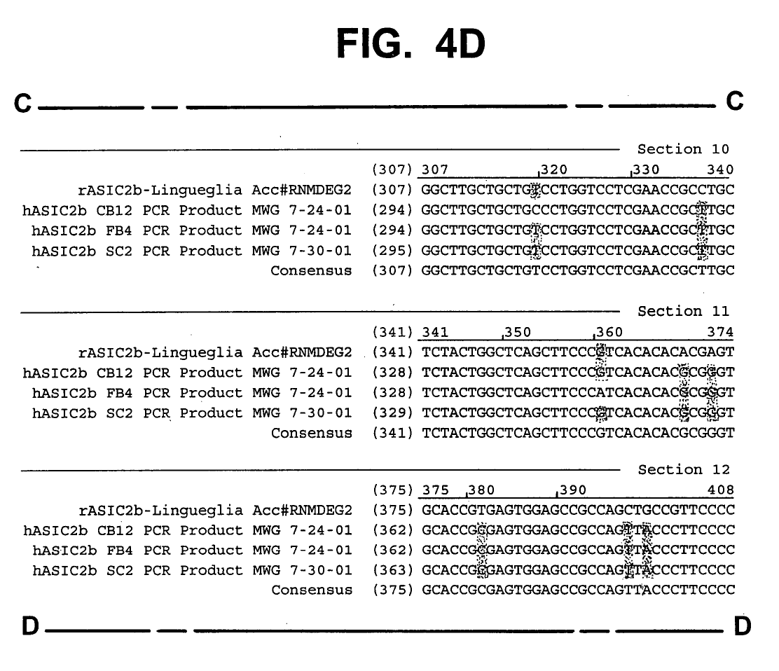

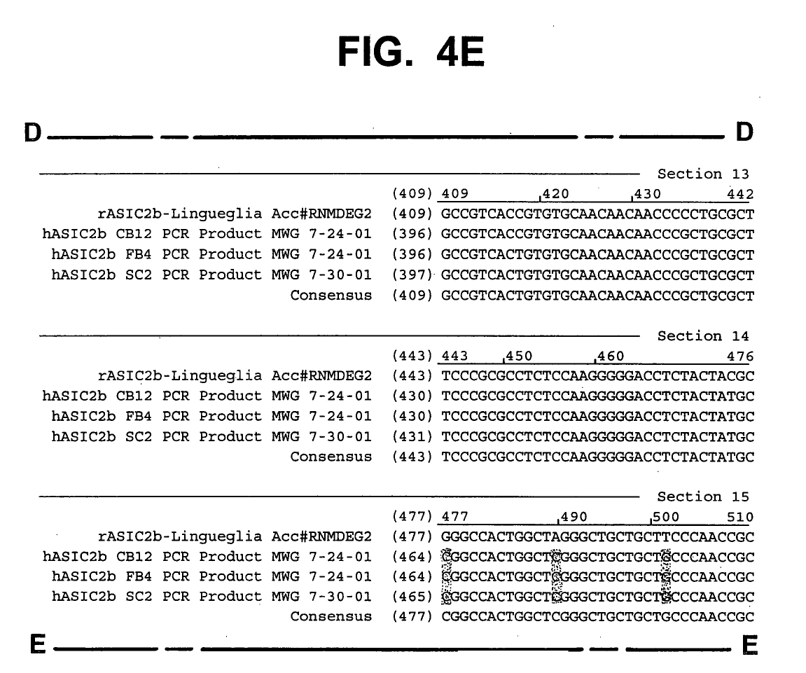

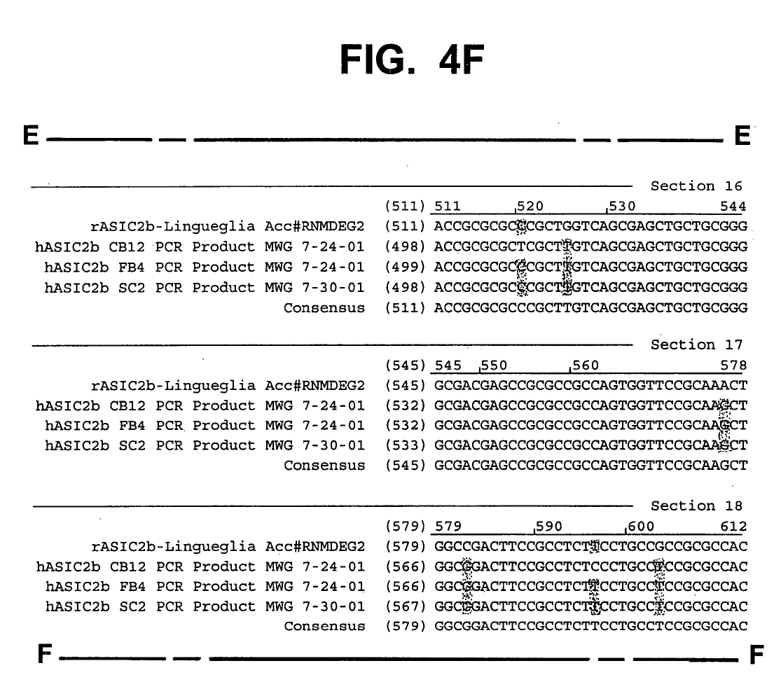

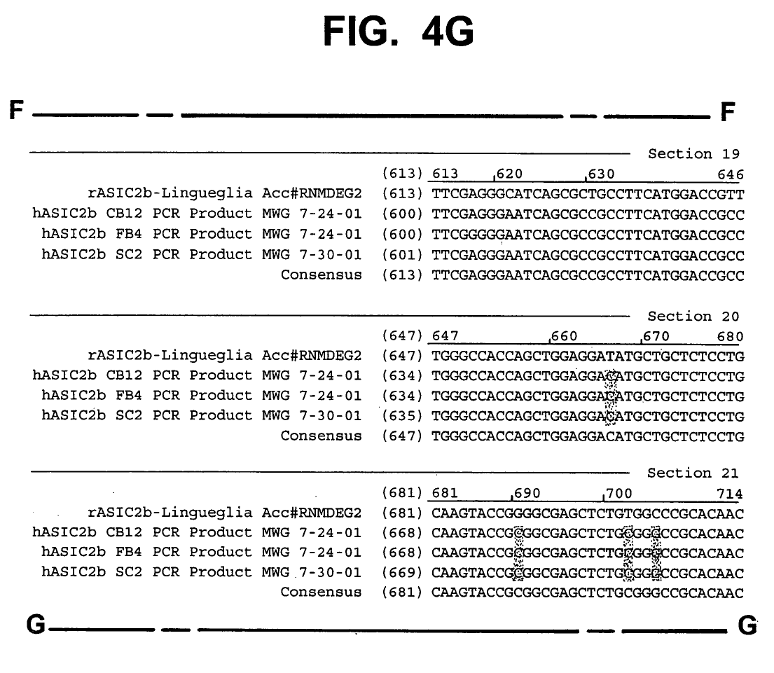

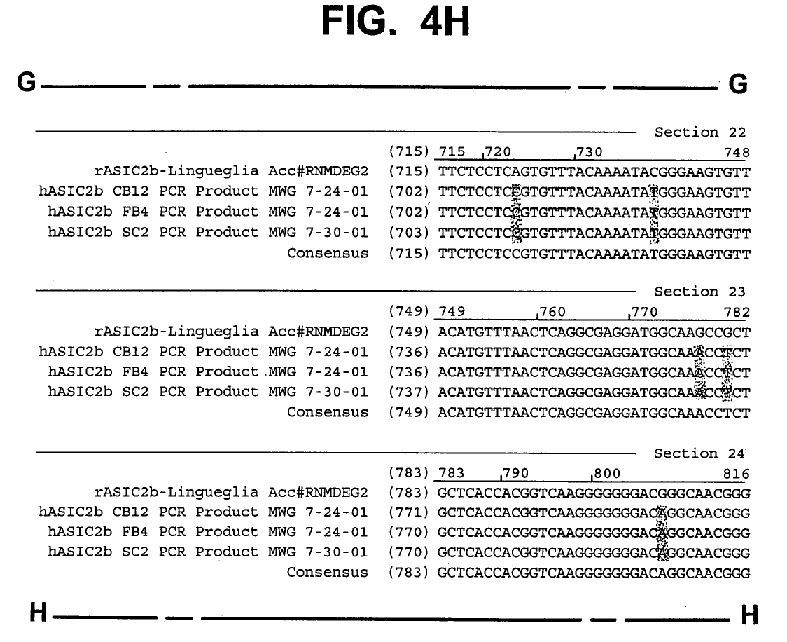

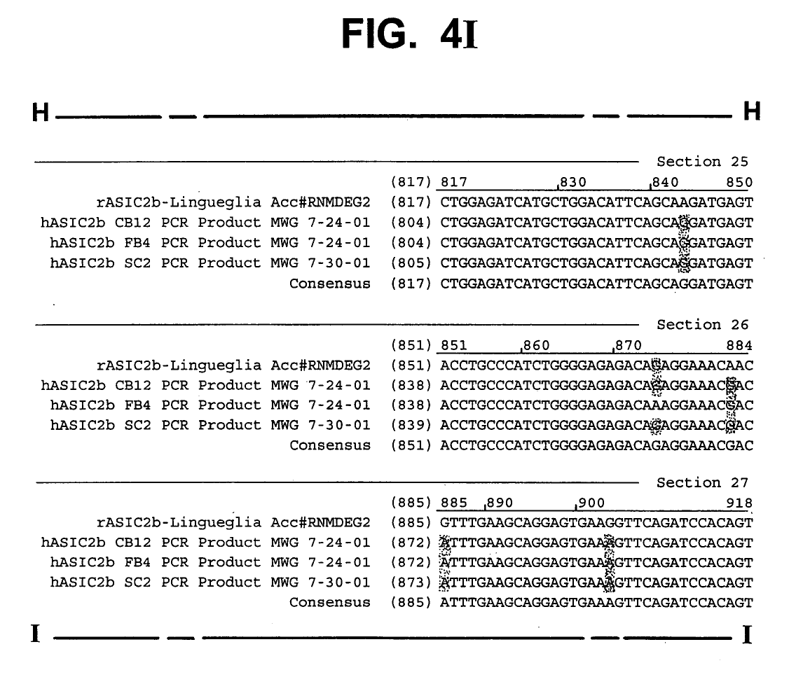

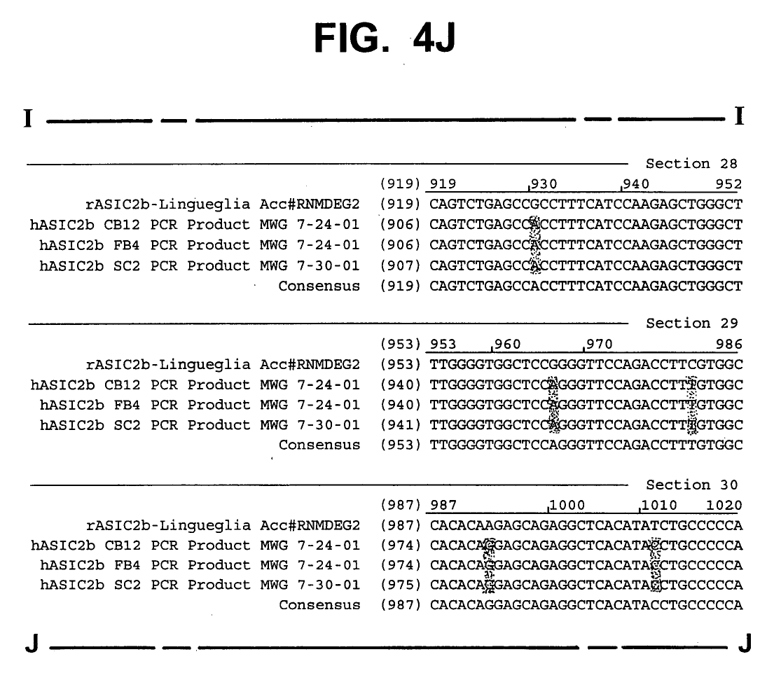

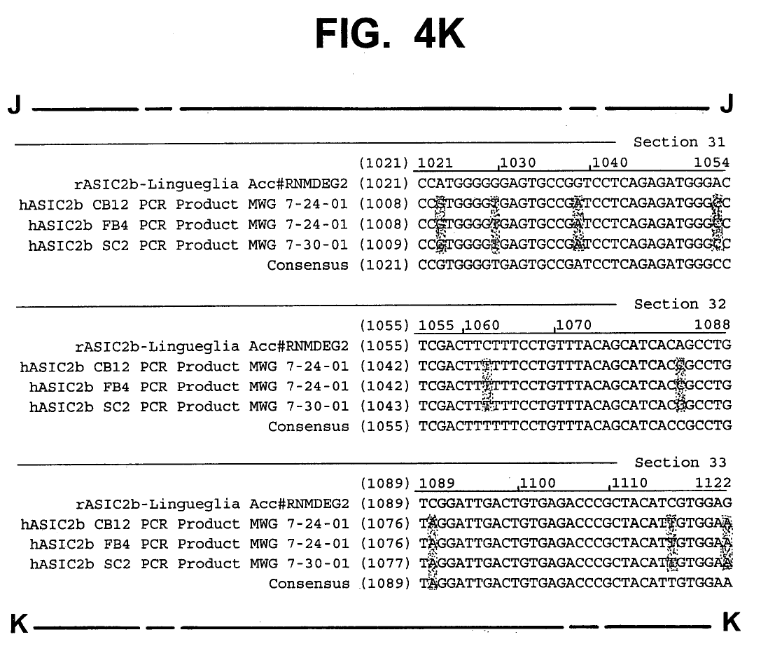

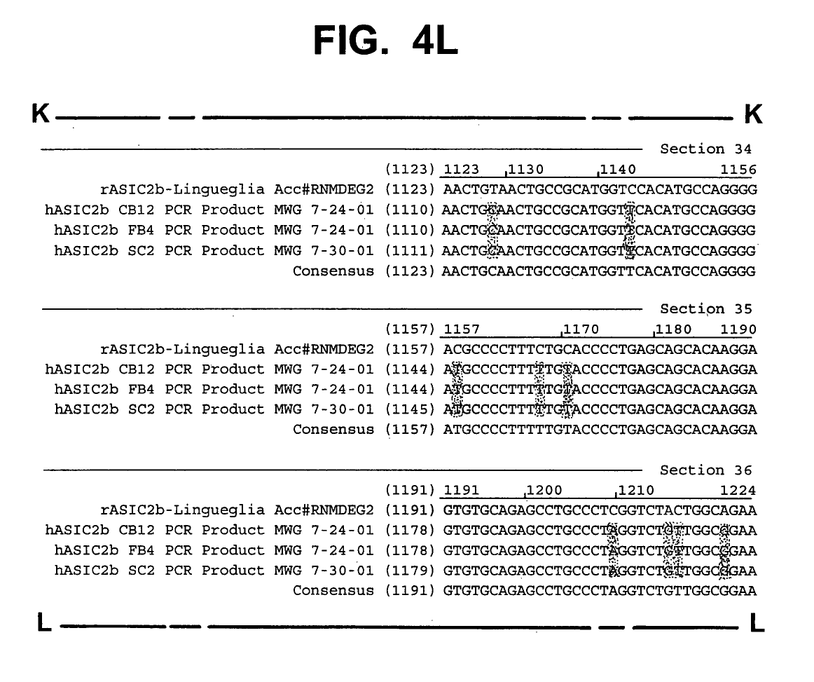

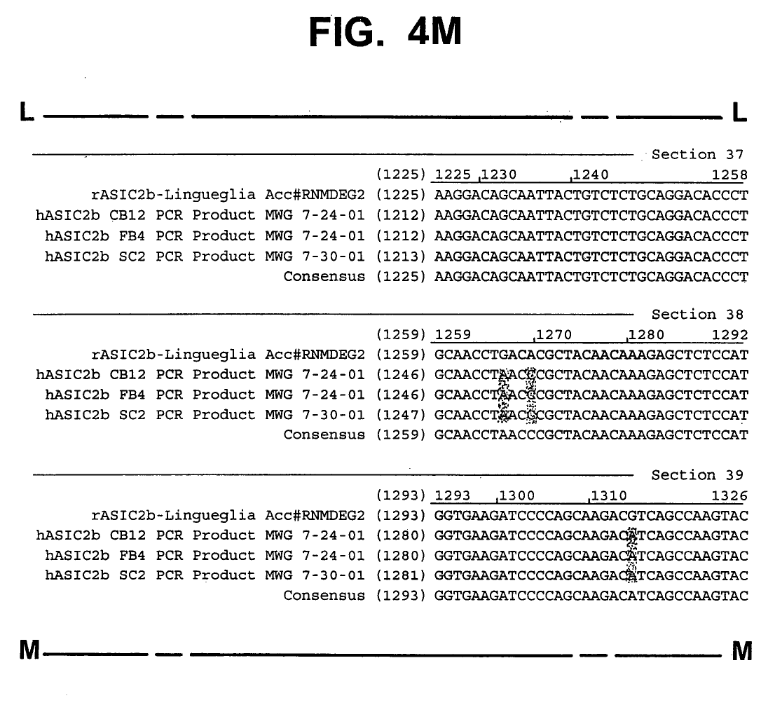

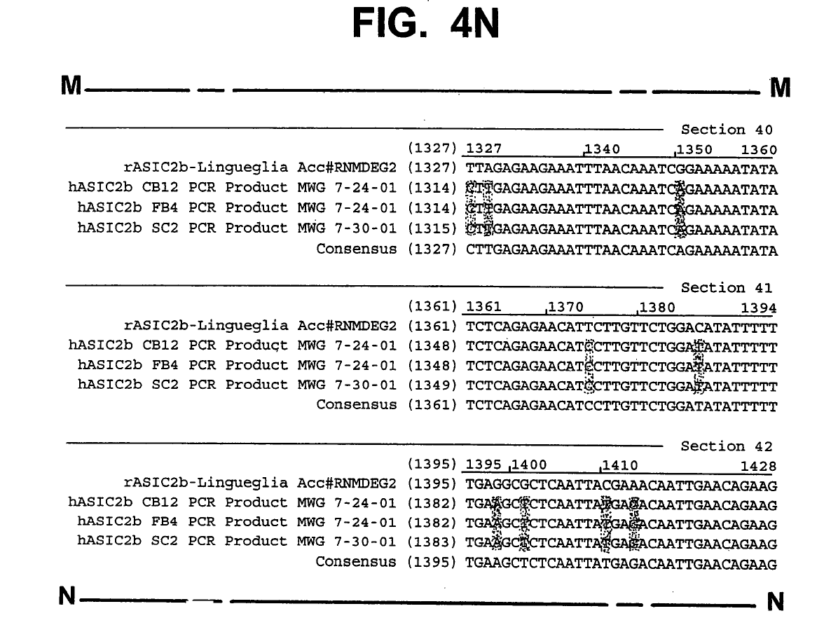

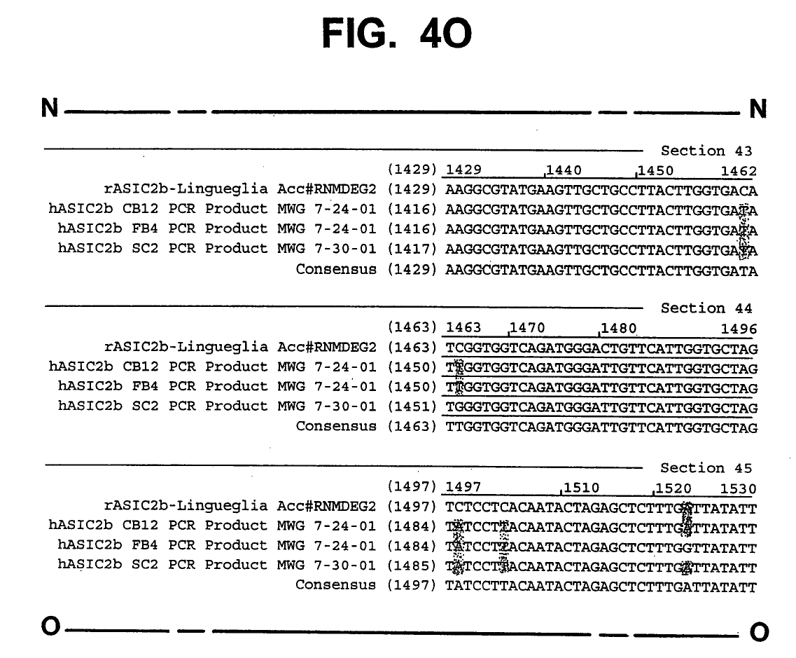

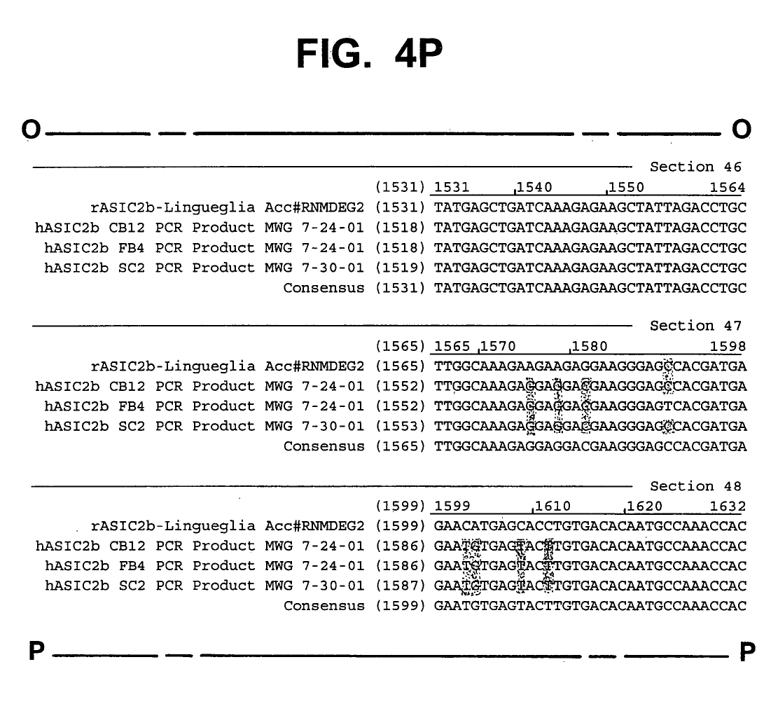

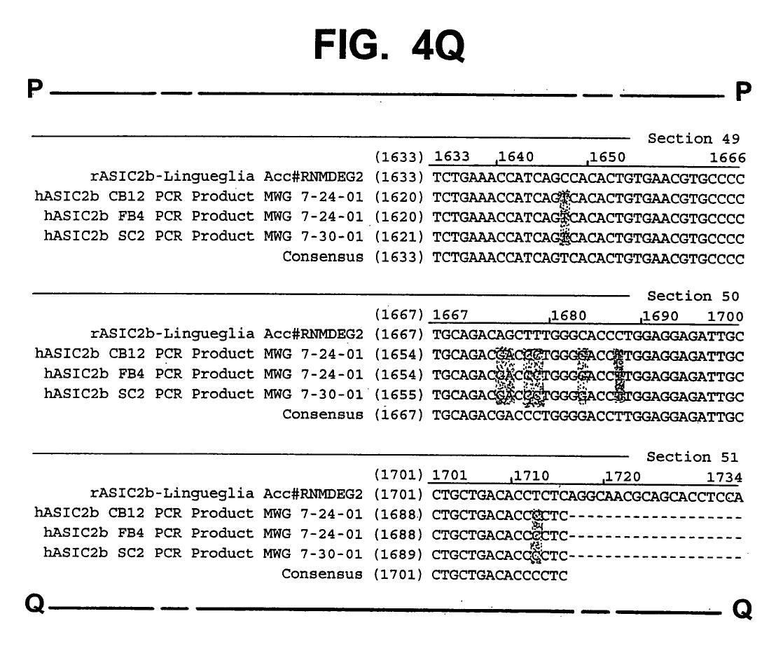

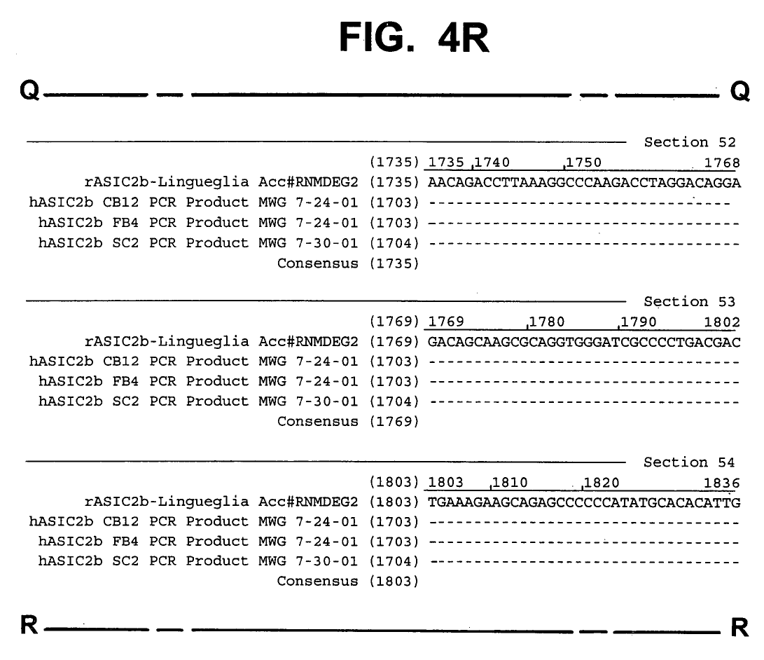

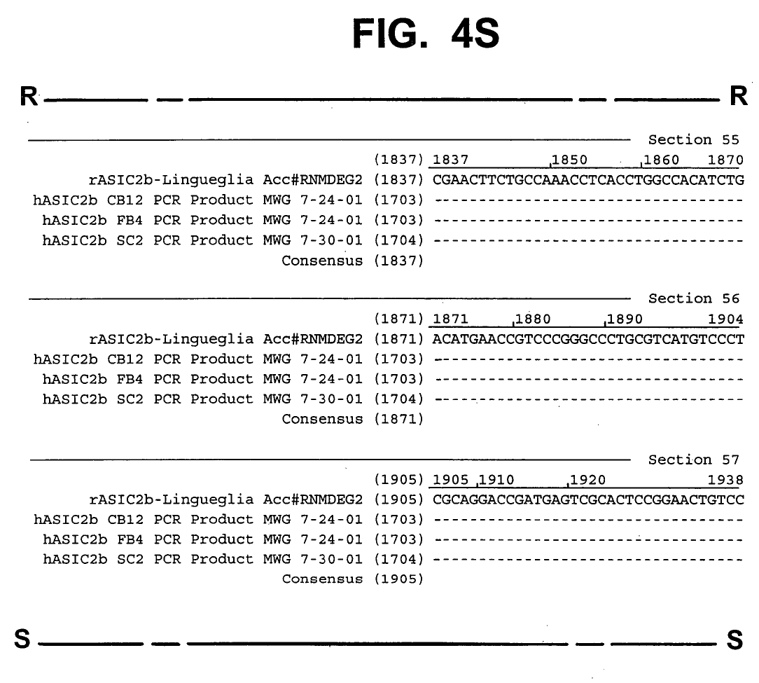

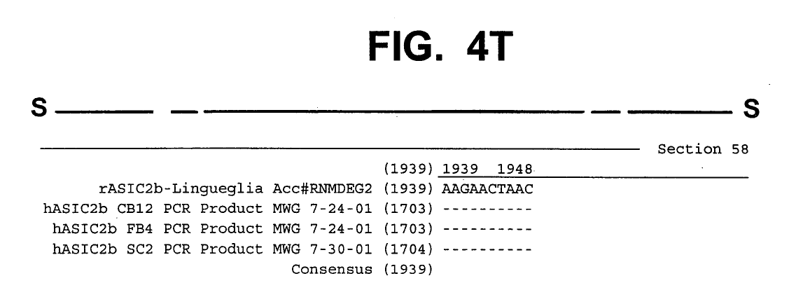

Portions of the cDNA and amino acid sequences of hASIC2b clones have about 91% sequence identity to the rat ASIC2b cDNA (SEQ ID NO:2) and amino acid (SEQ ID NO:3) sequences, and about 96% sequence identity to regions of the human ASIC2a cDNA (SEQ ID NO:4) and about 73% identity to the human ASIC2a amino acid sequence (SEQ ID NO:5) (hASIC2a sequences are described in GenBank Accession No. NM_{—}001094—see Results). See FIGS. 2-5.

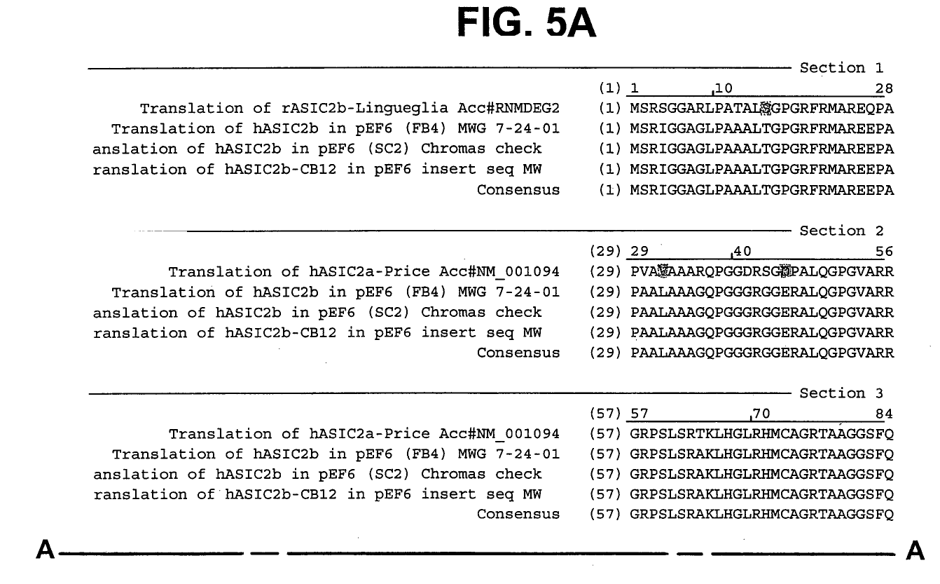

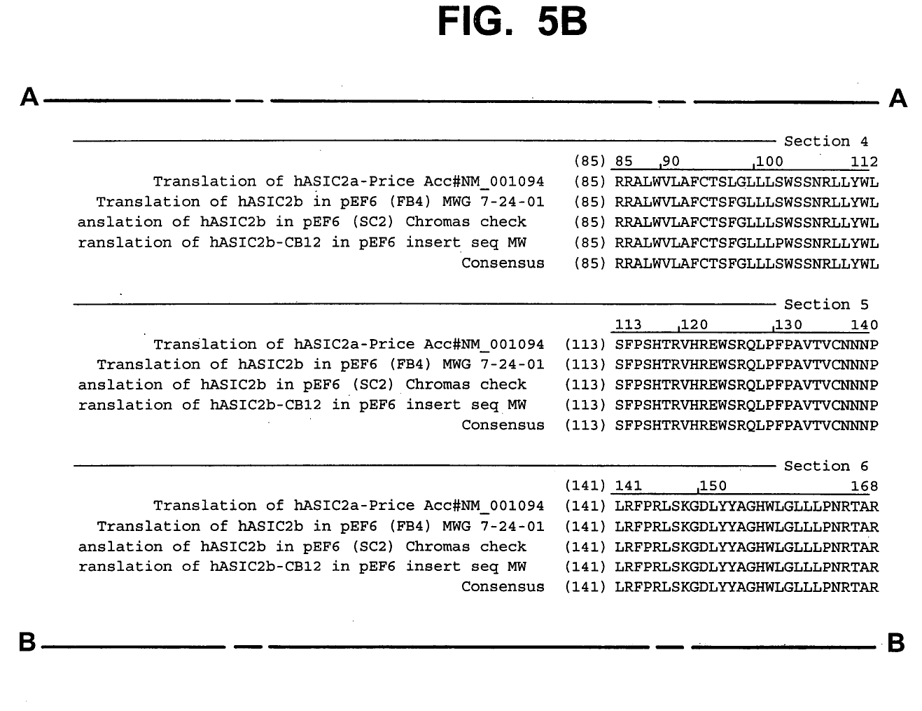

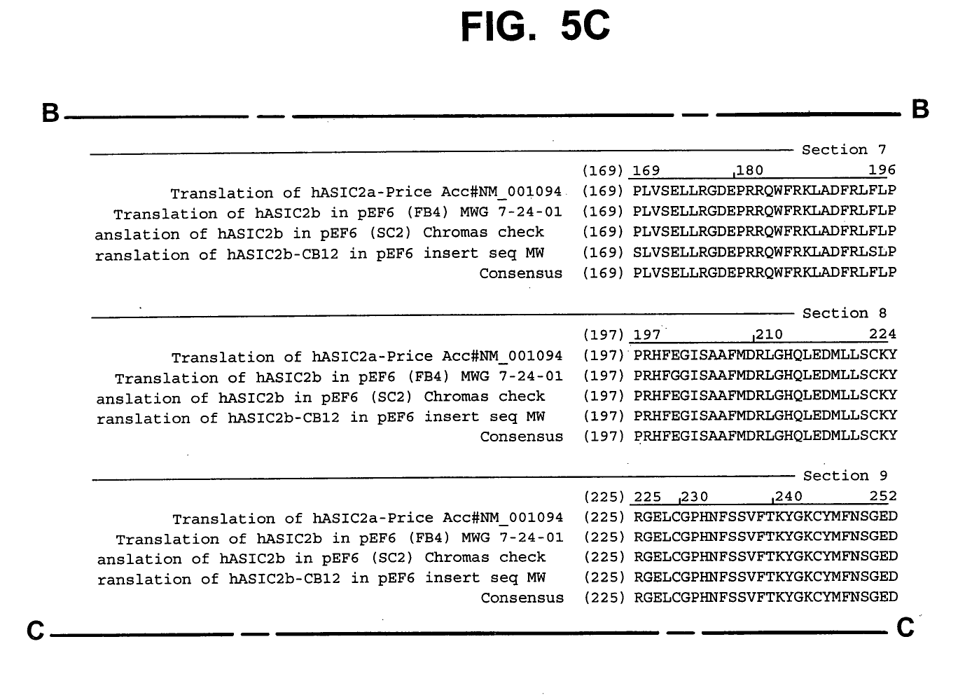

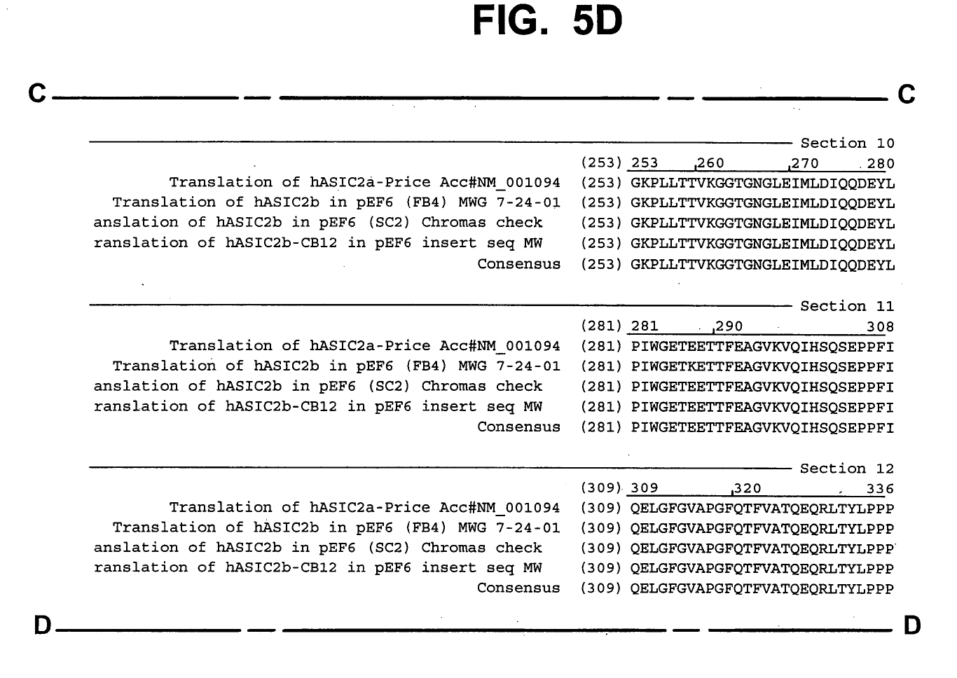

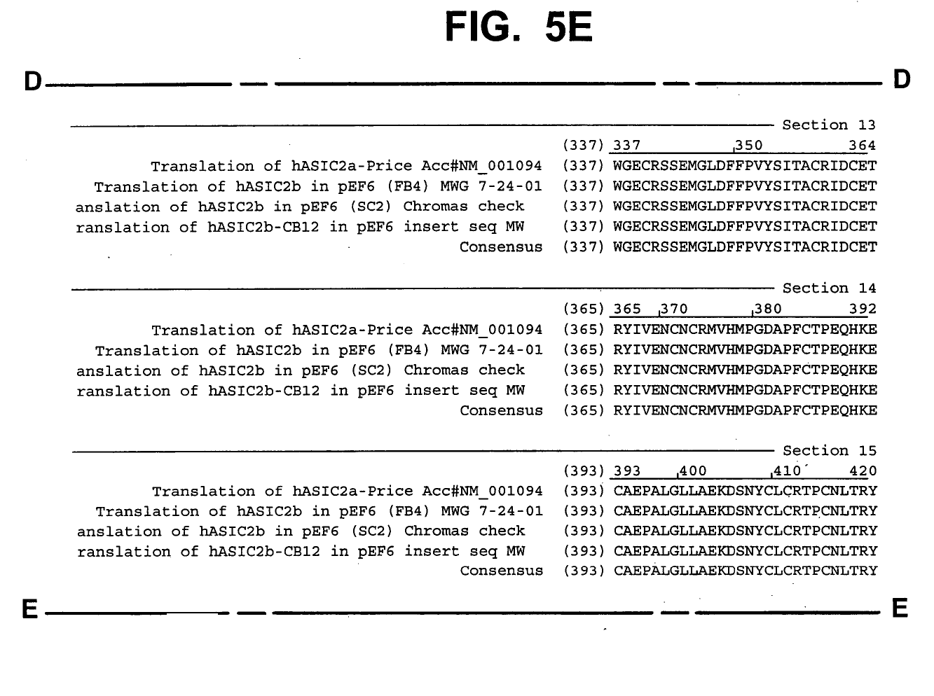

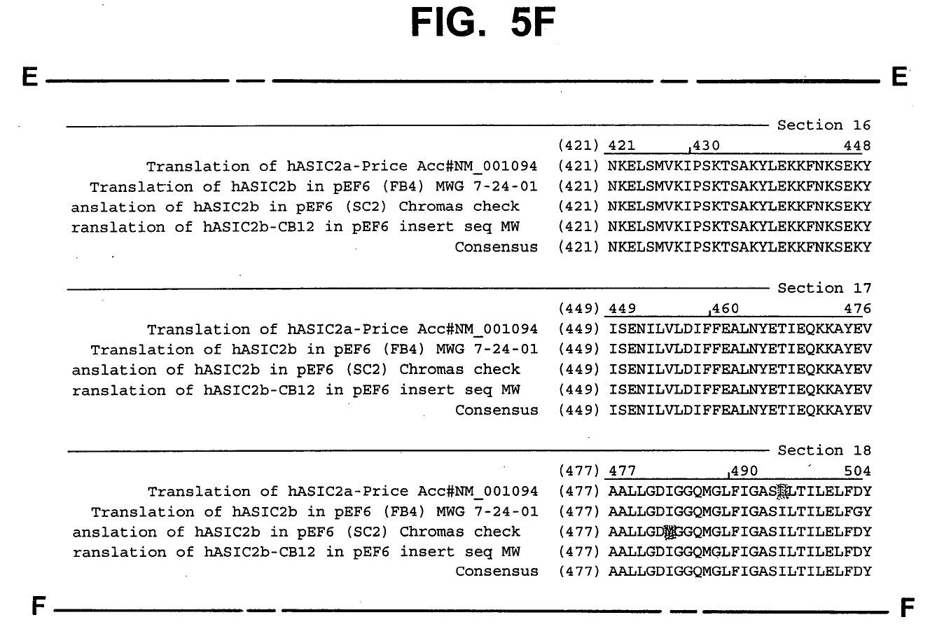

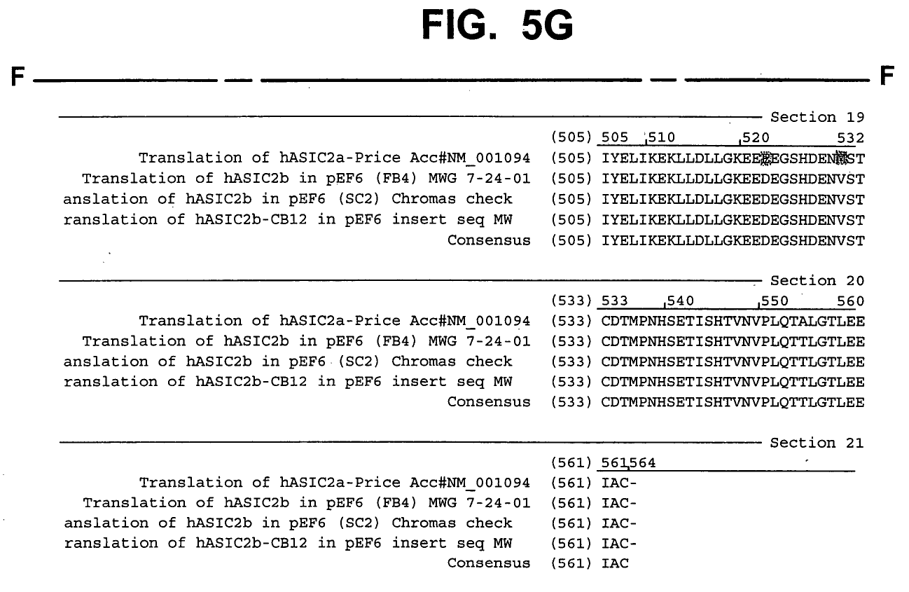

The three amino acid sequences cloned from three different tissues, cerebellum, fetal brain, and spinal cord of the present invention (designated CB12, FB4, and SC2 herein, SEQ ID NOs: 7, 9, and 11, respectively) exhibit differences among each other and/or from the rat ASIC2b ortholog at selected residues (See FIG. 5). The consensus sequence for SEQ ID NOs: 7, 9, and 11 is set forth in SEQ ID NO:14. For example, amino acid residue number 46 in FIG. 5 indicates the presence of a proline in the rat ASIC2b sequence, an arginine in the FB4 and SC2 clones, and a leucine in the CB12 clone. The differences among the three clones are unpredictable based on the rat ASIC2b amino acid sequence. Differences in the sequence among different tissues types within the CNS or PNS may result in differences in selectivity or activity of the hASIC2b-containing ion channel. The respective cDNA sequences, extending from the first residue of the start codon to the last residue before the stop codon, are depicted in SEQ ID NOS: 15, 16, and 17, for CB12, FB4, and SC2, respectively. An alternative SC2 protein sequence is set forth in SEQ ID NO:18, and the corresponding cDNA is set forth in SEQ ID NO:19.

Thus, the present invention advantageously provides hASIC2b subunit proteins, including fragments and derivatives thereof; hASIC2b-encoding nucleic acids, and portions thereof, including oligonucleotide primers and probes, and hASIC2b regulatory sequences (e.g., an hASIC2b promoter and splice sites with introns); hASIC2b subunit-specific antibodies; and related methods of using these materials to detect the presence of hASIC2b subunit proteins or nucleic acids.

The present invention also provides an assay method for screening to identify selective modulators of hASIC2b subunit-containing ion channels. The method involves detecting whether a test compound increases or decreases the activity of an hASIC2b subunit-containing ion channel, as determined, e.g., by measuring current phase and ion selectivity. The assay method is preferably conducted using at least one host cell that expresses or overexpresses functional acid sensing ion channels comprising at least one hASIC2b subunit protein of the invention, wherein the expression or overexpression of the channel results from genetic engineering. In one preferred embodiment, the test compound inhibits the pH-activated response of the ion channel. In another preferred embodiment, the test compound potentiates the pH-activated response of the ion channel. The test system preferably involves the use of an appropriate cell culture medium to permit cell growth and viability, as well as tissue culture plates or arrays containing the host cells in cell culture medium. In specific non-limiting embodiments, host cells are appropriate cells selected from a mammalian cell line, or from an vertebrate cell line such as, e.g., Xenopus.

### Definitions

General Definitions

The following definitions are provided for clarity and illustrative purposes only, and are not intended to limit the scope of the invention.

As use herein, the term “ion channel” refers to a transmembrane pore that presents a hydrophilic channel for specific ions to cross a lipid bilayer down their electrochemical gradients. In a preferred embodiment, the ion channel is a voltage-gated ion channel.

As used herein, the term “isolated” means that the referenced material is removed from the environment in which it is normally found. Thus, an isolated biological material can be free of cellular components, i.e., components of the cells in which the material is found or produced in nature. In the case of nucleic acid molecules, an isolated nucleic acid includes a PCR product, an mRNA, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more naturally occurring introns. Isolated nucleic acid molecules include sequences inserted into plasmids, cosmids, artificial chromosomes, phages and the like. Thus, in a specific embodiment, a recombinant nucleic acid is an isolated nucleic acid. An isolated protein may be associated with other proteins or nucleic acids, or both, with which it is associated in the cell, or with cellular membranes if it is a membrane-associated protein. A protein expressed from a vector in a cell, particularly a cell in which the protein is normally not expressed, is also a regarded as isolated. An isolated organelle, cell, or tissue is removed from the anatomical site in which it is naturally found in a cell or an organism. An isolated material may be, but need not be, purified. As used herein to refer to nucleic acids, the term “isolated” does not encompass man-made genomic or cDNA libraries.

The term “purified” as used herein refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material is substantially free of contaminants. Purity can be evaluated by chromatography, gel electrophoresis, immunoassay, composition analysis, biological assay, and other methods known in the art.

Methods for purification are well-known in the art. For example, nucleic acids can be purified by precipitation, chromatography (including preparative solid phase chromatography, oligonucleotide hybridization, and triple helix chromatography), ultracentrifugation, and other means. Polypeptides and proteins can be purified by various methods including, without limitation, preparative disc-gel electrophoresis, isoelectric focusing, HPLC, reversed-phase HPLC, gel filtration, ion exchange and partition chromatography, precipitation and salting-out chromatography, extraction, and countercurrent distribution. For some purposes, it is preferable to produce the polypeptide in a recombinant system in which the protein contains an additional sequence tag that facilitates purification, such as, but not limited to, a polyhistidine sequence (Hi®-tag-Novagen, Madison, Wis.), or a sequence that specifically binds to an antibody, such as the FLAG® tag (Sigma, St. Louis, Mo.), HA®-tag (Roche Diagnostics, Indianapolis, Ind.), or that can be column-purified, such as via glutathione S-transferase (GST-Amersham Biosciences, Piscataway, N.J.). The polypeptide can then be purified from a crude lysate of the host cell by chromatography on an appropriate solid-phase matrix. Alternatively, antibodies produced against the protein or against peptides derived therefrom can be used as purification reagents. Cells can be purified by various techniques, including centrifugation, matrix separation (e.g., nylon wool separation), panning and other immunoselection techniques, depletion (e.g., complement depletion of contaminating cells), and cell sorting (e.g., fluorescence activated cell sorting (FACS)). Other purification methods are possible. A purified material may contain less than about 50%, preferably less than about 75%, and most preferably less than about 90% by weight of the cellular components with which it was originally associated. The term “substantially pure” indicates the highest degree of purity which can be achieved using conventional purification techniques known in the art.

In a specific embodiment, the term “about” or “approximately” means plus or minus 10% of the stated numerical value or range.

A “sample” as used herein refers to a biological material that can be tested for the presence of an hASIC2b subunit-containing protein or hASIC2b subunit-encoding nucleic acid. Such samples can be obtained from animal, and preferably human, subjects and include tissue, especially brain tissue and neural tissue, and cell cultures of these tissues.

Non-human animals include, without limitation, laboratory animals such as mice, rats, rabbits, hamsters, guinea pigs, etc.; domestic animals such as dogs and cats; and farm animals such as sheep, goats, pigs, horses, and cows.

The term “modulator” refers to a compound that binds to an ion channel comprising a hASIC2b subunit of the invention, and differentially affects the activity of the ion channel in response to a decrease in extracellular pH, when compared to the activity of the same ion channel not contacted with the compound. Ion channel activity can be measured, e.g., using electrophysiology, or according to other known methods in the art.

The terms “inhibitor” and “antagonist” refer to a compound that binds to an ion channel comprising the hASIC2b subunit and blocks, inhibits, impedes, or reduces the activity of that channel, such as a functional effect elicited by the decrease in extracellular pH. The inhibitor or antagonist may bind directly to the hASIC2b subunit or to another subunit within the channel comprising the hASIC2b subunit.

As used herein the term “transfected cell” or “transformed cell” refers to a host cell that has been genetically engineered to express or overexpress a nucleic acid encoding a hASIC2b subunit, preferably in combination with one or more ASIC subunits such as, e.g., hASIC1 or hASIC2a (described in GenBank Accession Nos. XM_{—}018152, and NM_{—}001094, respectively). Any cell can be used, preferably a eukaryotic cell, and more preferably a vertebrate cell such as, e.g., a Xenopus cell, or a mammalian cell. Such cells additionally can be genetically engineered to coexpress or overexpress a different ASIC subunit. Such genetically engineered cells include those cells into which one or more heterologous ASIC-encoding nucleic acids have been introduced and are expressed or overexpressed. Such genetically engineered cells also include those cells engineered to express or overexpress one or more endogenous ASIC subunits, for example, by gene activation technology.

Such cells are particularly suitable to conduct an assay to screen for compounds that modulate the function of the hASIC2b subunit-containing ion channel in response to a decrease in extracellular pH. An “assay method” typically makes use of one or more such cells, e.g., in a microwell plate or some other culture system. The effects of a test compound can be determined on a single cell or on a collection of cells sufficient to allow measurement of ionic current, activation threshold, or ionic permeability characteristics of the hASIC2b subunit-containing ion channels. For example, single cells can be tested, e.g., by use of patch clamp or other appropriate electrophysiological techniques.

A “test compound” or “candidate compound” is any molecule that can be tested for its ability to bind to the hASIC2b subunit-containing ion channel, or a subunit thereof, and preferably modulate its effect on the hASIC2b subunit-containing ion channels, as set forth herein. A compound that binds and modulates a hASIC2b subunit-containing ion channel may be designated as a “lead compound” suitable for further testing and development.

The term “pain disorder” includes “chronic pain,” defined as pain lasting longer than one month (Bonica, Semin Anesth 1986, 5:82-99), and is characterized by unrelenting persistent pain that is not amenable to routine pain control methods. Chronic pain includes, but is not limited to, inflammatory pain, postoperative pain, cancer pain, osteoarthritis pain associated with metastatic cancer, trigeminal neuralgia, acute herpetic and postherpetic neuralgia, diabethic neuropathy, causalgia, brachial plexus avulsion, occipital neuralgia, reflex sympathetic dystrophy, fibromyalgia, gout, phantom limb pain, burn pain, pain associated with spinal cord injury, multiple sclerosis, reflex sympathetic dystrophy and lower back pain and other forms of neuralgia, neuropathic, and idiopathic pain syndromes.

“Pain disorder” also includes “neuropathic pain,” or pain caused by injury or infection of peripheral sensory nerves. It includes, but is not limited to pain from peripheral nerve trauma, herpes virus infection, diabetes mellitus, causalgia, plexus avulsion, neuroma, limb amputation, and vasculitis. Neuropathic pain is also caused by nerve damage from chronic alcoholism, human immunodeficiency virus infection, hypothyroidism, uremia, or vitamin deficiencies. Neuropathic pain includes but is not limited to pain caused by nerve injury such as, for example, the pain from which diabetics suffer.

Chronic and neuropathic types of pain generally arise from injury to the peripheral or central nervous tissue.

The term “pain disorder” further includes “nociceptive pain,” which is pain resulting from activation of pain-sensitive nerve fibers, either somatic or visceral. Nociceptive pain generally results as a response to direct tissue damage. The initial trauma causes the release of several chemicals including bradykinin, serotonin, substance P, histamine, and prostaglandin. When somatic nerves are involved, the pain is typically experienced as aching or pressure-like.

Molecular Biology—Definitions

In accordance with the present invention there may be employed conventional molecular biology, microbiology, and recombinant DNA techniques within the skill of the art. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

“Amplification” of DNA as used herein denotes the use of exponential amplification, techniques such as polymerase chain reaction (PCR), and non-exponential amplification, such as linked linear amplification, to increase the concentration of a particular DNA sequence within a mixture of DNA sequences. For a description of PCR see Saiki et al., Science 1988, 239:487. For a description of linked linear amplification, see U.S. Pat. Nos. 6,335,184 and 6,027,923 and Reyes et al., Clinical Chemistry, 2001;47: 131-40; Wu et al., Genomics, 1989;4: 560-569.

As used herein, “sequence-specific oligonucleotides” refers to related sets of oligonucleotides that can be used to detect allelic variations or mutations in the hASIC2b gene, or can be used for amplification of the hASIC2b gene.

The nucleic acid molecules (polynucleotides) described herein may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acid molecules may also be modified by many means known in the art. Non-limiting examples of such modifications include methylation, “caps”, substitution of one or more of the naturally occurring nucleotides with an analog, and internucleotide modifications such as, for example, replacement with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins or peptides (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, biotin, and the like.

A “coding sequence” or a sequence “encoding” an expression product, such as an RNA or polypeptide, is a nucleotide sequence that, when expressed, results directly or indirectly in the production of that RNA or polypeptide, i.e., the nucleotide sequence encodes an amino acid sequence for that polypeptide. A coding sequence or “open reading frame (ORF)” for a protein typically will include a start codon (usually ATG) and a stop codon.

The term “gene”, also called a “structural gene” refers to a basic unit of hereditary material. Specifically a gene is an ordered sequence of DNA nucleotide bases that encodes one polypeptide chain (via mRNA). The gene includes regions preceding and following the coding region (such as promoter sequences, a 5′-untranslated region, and a 3′-untranslated region, which affect, for example, the conditions under which the gene is expressed) as well as (in eukaryotes) intervening non-coding sequences (introns) between individual coding segments (exons).

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined, for example, by mapping with nuclease S1), as well as protein binding domains (consensus sequences) responsible for the binding of RNA polymerase. The present invention includes the hASIC2b subunit gene promoter found in the genome, which can be operatively associated with a hASIC2b coding sequence with a heterologous coding sequence.

The term “host cell” means any cell of any organism that is selected, modified, transformed, grown, or used or manipulated in any way, for the production of a substance by the cell, for example by the expression by the cell of a gene, a DNA or RNA sequence, or polypeptide. Host cells can further be used for screening or other assays, as described infra.

A coding sequence is “under the control of” or “operatively associated with” transcriptional and translational control sequences in a cell when such control sequences operate to effect RNA polymerase transcription of the coding sequence into mRNA, which is then trans-RNA spliced (if it contains introns) and translated, in the case of mRNA, into the protein encoded by the coding sequence.

The terms “express” and “expression” mean allowing or causing the information in a gene, cDNA, or mRNA sequence to become manifest, for example, by producing a protein by activating the cellular functions involved in transcription and translation of a corresponding gene, cDNA, or mRNA sequence. A gene or cDNA sequence is expressed in or by a cell to form an “expression product” such as a protein. The expression product itself, e.g., the resulting protein, may also be said to be “expressed” by the cell. An expression product can be characterized as intracellular, extracellular, transmembrane, or secreted. The hASIC2b subunit protein of the invention is typically expressed as a transmembrane protein with intracellular and extracellular domains.

The term “transfection” means the introduction of a “foreign” (i.e., extrinsic or extracellular) gene, DNA or RNA sequence into a host cell so that the host cell will express the introduced gene or sequence to produce a desired substance, typically a protein or enzyme encoded by the introduced gene or sequence. The introduced gene or sequence may also be called a “cloned” or “foreign” or “heterologous” gene or sequence, and may include regulatory or control sequences, such as start, stop, promoter, signal, secretion, or other sequences used by a cell's genetic machinery. The gene or sequence may include nonfunctional sequences or sequences with no known function.

The term “transformation” refers to the process by which DNA is introduced from the surrounding medium into a prokaryotic or eukaryotic host cell.

The term “transduction” refers to the introduction of DNA into a prokaryotic host cell via a bacterial virus, or bacteriophage.

A prokaryotic or eukaryotic host cell that receives and expresses introduced DNA or RNA has been “transformed” and is a “transformant” or a “clone.” The DNA or RNA introduced into a host cell can come from any source, including cells of the same genus or species as the host cell, or cells of a different genus or species, or synthetic sequences.

The term “recombinantly engineered cell” refers to any prokaryotic or eukaryotic cell that has been manipulated to express or overexpress hASIC2b by any appropriate method, including transfection, transformation or transduction. This term also includes endogenous activation or upregulation of a hASIC2b gene in a cell that does not normally express hASIC2b or that expresses the protein at a sub-optimal level.

The terms “vector”, “cloning vector” and “expression vector” mean the vehicle by which a DNA or RNA sequence (e.g., a foreign gene) can be introduced into a host cell, so as to transform the host and promote expression (e.g., transcription and translation) of the introduced sequence. Vectors include plasmids, phages, viruses, etc.; they are discussed in greater detail below.

Vectors typically comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites. A “cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell. A segment or sequence of DNA having inserted or added DNA, such as an expression vector, can also be called a “DNA construct.” A common type of vector is a “plasmid”, which generally is a self-contained molecule of double-stranded DNA, usually of bacterial origin, that can readily accept additional (foreign) DNA and which can readily introduced into a suitable host cell. A plasmid vector often contains coding DNA and promoter DNA and has one or more restriction sites suitable for inserting foreign DNA. Coding DNA is a DNA sequence that encodes a particular amino acid sequence for a particular protein or enzyme. Promoter DNA is a DNA sequence which initiates, regulates, or otherwise mediates or controls the expression of the coding DNA. Promoter DNA and coding DNA may be from the same gene or from different genes, and may be from the same or different organisms. A large number of vectors, including plasmid and fungal vectors, have been described for replication and/or expression in a variety of eukaryotic and prokaryotic hosts. Non-limiting examples include pKK plasmids (Clonetech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g., antibiotic resistance, and one or more expression cassettes.

The term “expression system” means a host cell and compatible vector under suitable conditions, e.g., for the expression of a protein coded for by foreign DNA carried by the vector and introduced to the host cell. Common expression systems include E. coli host cells and plasmid vectors, insect host cells and baculovirus vectors, and mammalian host cells and vectors.

The term “heterologous” refers to a combination of elements not naturally occurring together. For example, heterologous DNA refers to DNA not naturally present in that cell. Heterologous DNA refers to combinations of sequences that do not naturally occur together in that cell, e.g., promoter sequences from one cell type linked to coding sequences of a gene that is not normally controlled by that promoter, or in a different chromosomal site of the cell. Preferably, the heterologous DNA includes a gene foreign to the cell. A heterologous expression regulatory element is such an element operatively associated with a different gene than the one it is operatively associated with in nature. In the context of the present invention, a hASIC2b gene is heterologous to the vector DNA in which it is inserted for cloning or expression, and may be heterologous to a host cell containing such a vector, in which it is expressed.

The terms “mutant” and “mutation” mean any detectable change in genetic material, e.g., DNA, or any process, mechanism, or result of such a change. This includes gene mutations in which the structure (e.g., DNA sequence) of a gene is altered; any gene or DNA arising from any mutation process; and any altered expression product (e.g., protein or enzyme) expressed by a non-silent modification of a gene or DNA sequence. The term “variant” may also be used to indicate a modified or altered gene, DNA sequence, polypeptide, cell, etc., i.e., any kind of mutant resulting therefrom.

“Sequence-conservative variants” or “degenerate variants” of a polynucleotide sequence are those in which a change of one or more nucleotides in a given codon position results in no alteration in the amino acid encoded at that position.

“Function-conservative variants” are those in which a given amino acid residue in a protein or enzyme has been changed without substantially altering the function of the polypeptide, including, but not limited to, replacement of an amino acid residue with a residue having similar properties (such as, for example, polarity, hydrogen bonding potential, acidic, basic, hydrophobic, aromatic, and the like). Amino acid residues with similar properties are well known in the art. For example, arginine, histidine and lysine are hydrophilic-basic amino acids and may be interchangeable. Similarly, isoleucine, a hydrophobic amino acid, may be replaced with leucine, methionine or valine. Such changes are expected to have little or no effect on the apparent molecular weight or isoelectric point of the protein or polypeptide. Amino acids other than those indicated as conserved may differ in a protein or enzyme so that the percent protein or amino acid sequence identity between any two proteins of similar function may vary and may be, for example, at least 70%, 80%, 90%, 95% or 99%, as determined according to a default alignment scheme such as by the Cluster Method, wherein similarity is based on the MEGALIGN algorithm, or BLAST. As used herein, “function-conservative variants” do not include orthologs from different species.

“Allelic variant” refers to one of two or more alternate forms of a gene. For example, a nucleotide sequence on one chromosome that is found to differ by one or more nucleotides from the corresponding sequence found on the partner chromosome in the same cell, and from the known consensus sequence, is an allelic variant.

As used herein, the term “homologous” refers to the relationship between proteins that possess a “common evolutionary origin,” including proteins from superfamilies (e.g., the immunoglobulin superfamily) and homologous proteins from different species (e.g., myosin light chain, etc.) (Reeck et al., Cell 1987, 50:667). Such proteins (and their encoding genes) have sequence homology, as reflected by their sequence similarity or sequence identity, whether in terms of percent similarity or the presence of specific residues or motifs at conserved positions.

Accordingly, the term “sequence similarity” or “sequence identity” refers to the degree of identity or correspondence between nucleic acid or amino acid sequences at specific residues over the defined length of DNA or RNA or protein sequences that may or may not share a common evolutionary origin (see Reeck et al., supra). However, in common usage and in the instant application, the term “homologous,” when modified with an adverb such as “highly,” may refer to sequence similarity and may or may not relate to a common evolutionary origin.

In a specific embodiment, two DNA sequences are “substantially homologous” or “substantially similar” when at least about 80%, and most preferably at least about 90%, 95%, or 99% of the nucleotides match over the defined length of the DNA sequences, as determined by default sequence comparison algorithms, such as BLAST, FASTA, DNA Strider, etc. An example of such a sequence is an allelic or species variant of the specific hASIC2b gene of the invention. Sequences that are substantially homologous can be identified by comparing the sequences using standard software available in sequence data banks, or in a Southern hybridization experiment under, for example, stringent conditions as defined for that particular system.

Similarly, in a particular embodiment, two amino acid sequences are “substantially homologous” or “substantially similar” when greater than 80%, 90%, 95%, or 99% of the amino acids are identical. Preferably, the similar or homologous sequences are identified by alignment using, for example, the GCG (Genetics Computer Group, Program Manual for the GCG Package, Version 7, Madison, Wis.) pileup program, or any of the programs described above (BLAST, FASTA, etc).

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., supra). The conditions of temperature and ionic strength determine the “stringency” of the hybridization. For preliminary screening for homologous nucleic acids, low stringency hybridization conditions, using a Tm (melting temperature) in the range of about 55° C., with low-salt and/or denaturant concentrations, can be used, e.g., 5×SSC, 0.1% SDS, 0.25% milk, and no formamide; or 30% formamide, 5×SSC, 0.5% SDS. Moderate stringency hybridization conditions correspond to use of a higher Tm, such as, e.g., about 60° C., and higher concentrations of salt and/or denaturants e.g., 40% formamide, with 5× or 6×SSC. High stringency hybridization conditions correspond to the highest Tm, such as, e.g., about 65° C., and concentrations of denaturants, e.g., 50% formamide, 5× or 6×SSC. SSC is a 0.15M NaCl, 0.015M Na-citrate buffer. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the higher the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see Sambrook et al. 1989, supra, 9.50-9.51). For hybridization with shorter nucleic acids, i.e., oligonucleotides, the position of mismatches becomes more important, and the length of the oligonucleotide determines its specificity (see Sambrook et al.1989, supra, 11.7-11.8). A minimum length for a hybridizable nucleic acid is at least about 10 nucleotides; preferably at least about 15 nucleotides; and more preferably at least about 20 nucleotides.

In a specific embodiment, the term “standard hybridization conditions” refers to use of a Tm of about 55° C., and utilizes conditions as set forth above. In a preferred embodiment, the Tm is about 60° C.; in a more preferred embodiment, the Tm is about 65° C. In a specific embodiment, “high stringency” refers to hybridization and/or washing conditions at 68° C. in 0.2×SSC, at 42° C. in 50% formamide, 4×SSC, or under conditions that afford levels of hybridization equivalent to those observed under either of these two conditions.

As used herein, the term “oligonucleotide” refers to a nucleic acid, generally of at least 10, preferably at least 15, and more preferably at least 20 nucleotides, preferably no more than 100 nucleotides, that is hybridizable to a genomic DNA molecule, a cDNA molecule, or an mRNA molecule, or other nucleic acid of interest. Oligonucleotides can be labeled, e.g., with ³²P-nucleotides or nucleotides to which a label, such as biotin, has been covalently conjugated. In one embodiment, a labeled oligonucleotide can be used as a probe to detect the presence of a nucleic acid. In another embodiment, oligonucleotides (one or both of which may be labeled) can be used as PCR primers, either for cloning a full length nucleic acid or a fragment of a nucleic acid encoding the hASIC2b subunit, or to detect the presence of nucleic acids encoding hASIC2b. In a further embodiment, an oligonucleotide of the invention can form a triple helix with a hASIC2b-encoding DNA molecule. Generally, oligonucleotides are prepared synthetically, preferably on a nucleic acid synthesizer. Accordingly, oligonucleotides can be prepared with non-naturally occurring phosphoester analog bonds, such as thioester bonds, etc.

The present invention also provides antisense nucleic acids, which may be used to inhibit expression of hASIC2b of the invention. Inhibition of hASIC2b expression may be desired when upregulation of hASIC2b expression or excessive activation of an hASIC2b-containing ion channel induces or otherwise contributes to an increase in nociceptive pain resulting, e.g., from tissue acidosis or inflammation.

An “antisense nucleic acid” is a single stranded nucleic acid molecule, which may be DNA, RNA, a DNA-RNA chimera, or derivatives thereof, which, on hybridizing under cytoplasmic conditions with complementary bases in an RNA or DNA molecule, inhibits the expression or translation of the encoded gene. If the RNA is an mRNA transcript, the antisense nucleic acid is a counter-transcript or mRNA-interfering complementary nucleic acid. As presently used, “antisense” broadly includes RNA-RNA interactions, RNA-DNA interactions, and RNase-H mediated arrest. Antisense nucleic acid molecules can be encoded by a recombinant gene for expression in a cell (e.g., U.S. Pat. No. 5,814,500; U.S. Pat. No. 5,811,234), or alternatively they can be prepared synthetically (see, e.g., U.S. Pat. No. 5,780,607).

In addition to antisense sequences, the present invention also provides ribozymes and RNA interference sequences useful to inhibit hASIC2b expression. Ribozyme technology is described further in “Intracellular Ribozyme Applications: Principals and Protocols”, Ed. Rossi and Couture, 1999, Horizon Scientific Press. The term “RNA interference” or “RNAi” refers to the ability of double stranded RNA (dsRNA) to suppress the expression of a specific gene of interest in a homology-dependent manner. It is currently believed that RNA interference acts post-transcriptionally by targeting mRNA molecules for degradation. RNA interference commonly involves the use of dsRNAs that are greater than 50 bp; however, it can also be mediated through small interfering RNAs (siRNAs) or small hairpin RNAs (shRNAs), which are typically greater than 18 nucleotides in length. For reviews, see Bosner and Labouesse, Nature Cell Biol. 2000; 2: E31-E36 and Sharp and Zamore, Science 2000; 287: 2431-2433.

As used herein, the term “ligand” applies to any binding compound or ligand or their competitors and includes, but is not necessarily limited to the functional categories of agonist, partial agonist and antagonist. An “agonist” is defined as a ligand that promotes or enhances the normal biological function of the subunit or channel which contains hASIC2b. A “partial agonist” binds as does the agonist, but promotes only partial function. An “antagonist” inhibits all or partial channel function with its binding to a subunit of the channel which comprises hASIC2b in response to channel activation.

hASIC2b Nucleic Acids

A polynucleotide molecule encoding hASIC2b, whether genomic DNA or cDNA, can be isolated from any source, particularly from a human genomic or cDNA library. Methods for obtaining specific polynucleotide molecules are well known in the art, as described above (see, e.g., Sambrook et al., 1989, supra). The DNA may be obtained by standard procedures known in the art from cloned DNA (e.g., a DNA “library”), and preferably is obtained from a cDNA library prepared from tissues with high level expression of the encoded protein, or by chemical synthesis, or by cDNA cloning, or by the cloning of genomic DNA, or fragments thereof, purified from the desired cell (See, for example, Sambrook et al., 1989, supra; Glover, D. M. (ed.), 1985, DNA Cloning: A Practical Approach, MRL Press, Ltd., Oxford, U.K. Vol. I, II). Clones derived from genomic DNA may contain regulatory and intron DNA regions in addition to coding regions. Clones derived from cDNA will not contain intron sequences. Whatever the source, the polynucleotide molecule should be cloned into a vector suitable for propagation. Identification of a specific DNA fragment containing the desired hASIC2b-encoding sequence may be accomplished in a number of ways. For example, a portion of a hASIC2b gene exemplified infra can be purified and labeled to prepare a labeled probe, and a DNA library may be screened by nucleic acid hybridization to the labeled probe (Benton and Davis, Science 1977, 196:180; Grunstein and Hogness, Proc. Natl. Acad. Sci. U.S.A. 1975, 72:3961). Those DNA fragments with substantial homology to the probe, such as an allelic variant from another individual, will hybridize. In a specific embodiment, highest stringency hybridization conditions are used to identify a homologous hASIC2b gene.

Further selection can be carried out on the basis of the properties of the nucleic acid, e.g., if the nucleic acid encodes a protein product having the same physico-chemical profile (i.e., isoelectric, electrophoretic, electrophysiological, amino acid composition, partial or complete amino acid sequence, antibody binding activity, or ligand binding profile) of the hASIC2b subunit protein disclosed herein. Thus, the presence of the nucleic acid may be detected by assays based on the physical, chemical, immunological, or functional properties of its expressed product.

Other DNA sequences which encode substantially the same amino acid sequence as a hASIC2b gene may be used in the practice of the present invention. These include but are not limited to allelic variants, species variants, sequence conservative variants, and function conservative variants.

Amino acid substitutions may also be introduced to substitute an amino acid with a particularly preferable property. For example, a Cys may be introduced at a potential site to allow for disulfide bridges with another Cys.

The polynucleotide molecules encoding hASIC2b, and the encoded polypeptide, derivatives and analogs thereof, can be produced by various methods known in the art. The manipulations which result in their production can occur at the gene or protein level. For example, the cloned hASIC2b gene or cDNA sequence can be modified by any of numerous strategies known in the art (Sambrook et al., 1989, supra). The sequence can be cleaved at appropriate sites with restriction endonuclease(s), followed by further enzymatic modification if desired, isolated, and ligated in vitro. In the production of a polynucleotide molecule encoding a derivative or analog of hASIC2b, care should be taken to ensure that the sequence of the modified polynucleotide molecule remains within the same translational reading frame as the hASIC2b gene, uninterrupted by premature translational stop signals.

Additionally, the encoding nucleic acid sequence can be mutated in vitro or in vivo to create and/or destroy translation, initiation, and/or termination sequences, or to create variations in coding regions and/or form new restriction endonuclease sites or destroy preexisting ones, to facilitate further in vitro modification. Such modifications can be made to introduce restriction sites and facilitate cloning the polynucleotide molecule into an expression vector. Any technique for mutagenesis known in the art can be used, including but not limited to, in vitro site-directed mutagenesis (Hutchinson, C., et al., J. Biol. Chem., 1978;253:6551; Zoller and Smith, DNA, 1984;3:479-488; Oliphant et al., Gene 1986;44:177; Hutchinson et al., Proc. Natl. Acad. Sci. U.S.A., 1986;83:710), use of TAB linkers (Pharmacia), etc. PCR techniques are preferred for site directed mutagenesis (see Higuchi, 1989, “Using PCR to Engineer DNA”, in PCR Technology: Principles and Applications for DNA Amplification, H. Erlich, ed., Stockton Press, Chapter 6, pp. 61-70).

The identified and isolated polynucleotide molecule can then be inserted into an appropriate cloning vector. A large number of vector-host systems known in the art may be used. Possible vectors include, but are not limited to, plasmids or modified viruses, but the vector system must be compatible with the host cell used. Examples of vectors include, but are not limited to, E. coli, bacteriophages such as lambda derivatives, or plasmids such as Bluescript, pBR322 derivatives or pUC plasmid derivatives, e.g., pGEX vectors, pmal-c, pFLAG, etc. The insertion into a cloning vector can, for example, be accomplished by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any restriction site desired may be produced by ligating nucleotide sequences (linkers) onto the DNA termini; these ligated linkers may comprise specific chemically synthesized oligonucleotides encoding restriction endonuclease recognition sequences. In addition, simple PCR or overlapping PCR may be used to insert a fragment into a cloning vector.

Recombinant molecules can be introduced into host cells via transformation, transfection, infection, electroporation, etc., so that many copies of the gene sequence are generated, resulting in a high likelihood for protein expression. Preferably, the cloned gene is contained on a shuttle vector plasmid, which provides for propagation in a cloning cell, e.g., E. coli, and facile purification for subsequent insertion into an appropriate expression cell line, if such is desired. For example, a shuttle vector, which is a vector that can replicate in more than one type of organism, can be prepared for replication in both E. coli and Saccharomyces cerevisiae by linking sequences from an E. coli plasmid with sequences from the yeast 2μplasmid.

hASIC2b Regulatory Nucleic Acids

Elements of the hASIC2b promoter can be identified by scanning the human genomic region upstream of the hASIC2b start site, e.g., by creating deletion mutants and checking for expression, or by using an algorithm. Sequences up to about 6 kilobases (kb) or more upstream from the hASIC2b start site may contain tissue-specific regulatory elements.

The term “hASIC2b promoter” encompasses artificial or heterologous promoters. Such promoters can be prepared by deleting nonessential intervening sequences from the upstream region of the hASIC2b promoter, or by joining upstream regulatory elements from the hASIC2b promoter with a heterologous minimal promoter, such as the CMV immediate early promoter.

A hASIC2b promoter or expression control sequence can be operably associated with a heterologous coding sequence, e.g., for a reporter gene (luciferase and green fluorescent proteins are examples of reporter genes) in a construct. This construct can be used to test for conditions or reagents that normally result in hASIC2b expression. This construct can be used in screening assays, described below, for hASIC2b agonists and antagonists.

hASIC2b regulatory nucleic acids of the present invention may also include e.g., non-endogenous or artificial promoter sequences or encode zinc finger proteins that may be used, e.g., in gene activation techniques, to initiate expression of the hASIC2b subunit protein in cells where it is not normally expressed, or to upregulate expression of the hASIC2b subunit protein to a higher level. Gene activation techniques that may be adapted to this use are described in the art, e.g., in U.S. Pat. Nos. 5,641,670, 6,214,622, and 6,270,989 to Treco et al.

Expression of hASIC2b Polypeptides

The nucleotide sequence coding for hASIC2b, or an antigenic fragment, derivative or analog thereof, or a functionally active derivative thereof, including a chimeric protein, thereof, can be inserted into an appropriate expression vector, i.e., a vector which contains the necessary elements for the transcription and translation of the inserted protein-coding sequence. Thus, a nucleic acid encoding hASIC2b of the invention can be operationally associated with a promoter in an expression vector of the invention. Both cDNA and genomic sequences can be cloned and expressed under control of such regulatory sequences. Such vectors can be used to express functional or functionally inactivated hASIC2b polypeptides.

The necessary transcriptional and translational signals can be provided on a recombinant expression vector, or they may be supplied by the native gene encoding hASIC2b and/or its flanking regions.

Potential host-vector expression systems include but are not limited to mammalian cell systems transfected with expression plasmids or infected with virus (e.g., vaccinia virus, adenovirus, adeno-associated virus, herpes virus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

Expression of hASIC2b protein may be controlled by any promoter/enhancer element known in the art, but these regulatory elements must be functional in the host selected for expression. Promoters which may be used to control hASIC2b gene expression include, but are not limited to, cytomegalovirus (CMV) promoter (U.S. Pat. Nos. 5,385,839 and 5,168,062), the SV40 early promoter region (Benoist and Chambon, Nature, 1981;290:304-310), the promoter contained in the 3′ long terminal repeat of Rous sarcoma virus (Yamamoto, et al., Cell, 1980;22:787-797), the herpes thymidine kinase promoter (Wagner et al., Proc. Natl. Acad. Sci. U.S.A., 1981;78:1441-1445), the regulatory sequences of the metallothionein gene (Brinster et al., Nature, 1982;296:39-42); prokaryotic expression vectors such as the β-lactamase promoter (Villa-Komaroff, et al., Proc. Natl. Acad. Sci. U.S.A., 1978;75:3727-3731), or the tac promoter (DeBoer, et al., Proc. Natl. Acad. Sci. U.S.A., 1983;80:21-25); see also “Useful proteins from recombinant bacteria” in Scientific American, 242:74-94, 1980; promoter elements from yeast or other fungi such as the Gal 4 promoter, the ADC (alcohol dehydrogenase) promoter, PGK (phosphoglycerol kinase) promoter, alkaline phosphatase promoter; and transcriptional control regions that exhibit tissue specificity, such as, e.g., endothelial cell-specific promoters.

Solubilized forms of the protein can be obtained where necessary by solubilizing inclusion bodies or reconstituting membrane components, e.g., by treatment with detergent and, if desired, sonication or other mechanical processes, as described above. The solubilized protein can be isolated using various techniques, such as polyacrylamide gel electrophoresis (PAGE), isoelectric focusing, 2-dimensional gel electrophoresis, chromatography (e.g., ion exchange, affinity, immunoaffinity, and sizing column chromatography), centrifugation, differential solubility, immunoprecipitation, or by any other standard technique for the purification of proteins.

In a preferred embodiment, the hASIC2b is expressed in combination with an a-type subunit such as hASIC1, hASIC2a, hASIC3 or hASIC4 (described in GenBank Accession Nos. XM_{—}018152, NM_{—}001094, AF095897, and AJ271643, respectively). Cloning of hASIC1 and hASIC2a is described in Waldmann et al., Nature, 1997;86:17-177 and Lingueglia et al., Journal of Biological Chemistry, 1997;272:29778-83, respectively. Cloning of hASIC3 and hASIC4 is described U.S. Pat. No. 6,287,859 to DeWeille et al. and in published PCT application WO 01/66125 to Dubin et al. Expression of multiple subunits in mammalian cells can be achieved by IRES vectors or cotransfection/selection of several vectors under different selection pressures.

hASIC2b Binding Partners

The present invention further provides a method for identifying physiological binding partners of hASIC2b. One method for evaluating and identifying hASIC2b binding partners is the yeast two-hybrid screen. Preferably, the yeast two-hybrid screen is performed using a cell library with yeast that are transformed with recombinant hASIC2b. Alternatively, hASIC2b can be used as a capture or affinity purification reagent. In another alternative, labeled hASIC2b can be used as a probe for binding, e.g., by immunoprecipitation or Western analysis. Expected hASIC2b binding partners include other human ASIC subunits such as hASIC1, hASIC2a and hASIC3.

Generally, binding interactions between hASIC2b and any of its binding partners will be strongest under conditions approximating those found in the native cell, i.e., physiological conditions of ionic strength, pH and temperature. Perturbation of these conditions will tend to disrupt the stability of a binding interaction.

Antibodies to hASIC2b

Antibodies to hASIC2b are useful, inter alia, for determining the presence of hASIC2b in a cell and for cellular regulation (i.e., inhibition) of hASIC2b activity, as set forth below. According to the invention, a hASIC2b polypeptide produced recombinantly or by chemical synthesis, and fragments or other derivatives or analogs thereof, including fusion proteins, may be used as immunogens to generate antibodies that recognize the hASIC2b polypeptide. Such antibodies include but are not limited to polyclonal, monoclonal, chimeric, single chain, Fab fragments, and Fab expression libraries. Such an antibody binds specifically to human hASIC2b, and may recognize either a mutant form of hASIC2b or wild-type hASIC2b, or both. The antibodies of the present invention are specific for hASIC2b and either do not recognize, or bind with lower affinity to, orthologs. Specific binding of such antibodies to hASIC2b polypeptides will inhibit the activity of hASIC2b, or an ion channel comprising hASIC2b.

Various procedures known in the art may be used for the production of antibodies to hASIC2b polypeptide or derivative or analog thereof. These include but are not limited to the hybridoma technique originally developed by Kohler and Milstein (Nature, 1975;256:495-497), as well as the trioma technique, the human B-cell hybridoma technique (Kozbor et al., Immunology Today, 1983;4:72; Cote et al., Proc. Natl. Acad. Sci., 1983;80:2026-2030), and the EBV-hybridoma technique to produce human monoclonal antibodies (Cole et al., in Monoclonal Antibodies and Cancer Therapy, Alan R. Liss, Inc., 1985, pp. 77-96).

For purposes of detecting the presence of hASIC2b, it is preferred that the antibody is labeled. The term “labeled,” with regard to the antibody, is intended to encompass direct labeling of the antibody by coupling (i.e., physically linking) a detectable substance to the antibody, as well as indirect labeling of the antibody by reactivity with another reagent that is directly labeled. An example of indirect labeling includes detection of a primary antibody using a fluorescently labeled secondary antibody. Thus, the detection method of the invention can be used to detect hASIC2b subunit protein in a biological sample or in host cells in vitro. For example, in vitro techniques for detection of hASIC2b subunit protein include enzyme linked immunosorbent assays (ELISAs), Western blots, immunoprecipitations and immunofluorescence.

Agonists and Antagonists

The present invention also contemplates the identification of compounds that modulate hASIC2b sodium channel activation and activity. Such compounds are useful, e.g., for inhibiting (i.e., antagonizing) or increasing (i.e., agonizing) biological activities that are associated with ion channel activation and/or as therapeutic agents for treating disorders associated with excessive sodium channel activation.

Compounds that modulate hASIC2b activity or an activity associated therewith may be readily identified using screening methods of the present invention. In one embodiment, compounds identified by the screening methods of this invention bind to an hASIC2b subunit-containing ion channel. Compounds identified by the present method may antagonize or agonize hASIC2b subunit-containing channel activity, as well as a related downstream biological effect (e.g., the ability of DRG to transmit nociceptive signals from the peripheral nervous system (PNS) to the central nervous system (CNS)) associated with excessive hASIC2b subunit-containing channel activity.

In vivo or cell culture assays may be used to determine whether a test compound functions as an antagonist to inhibit hASIC2b subunit-containing ion channel activity in cells. For instance, cell culture assays may be used to measure a test compound's ability to modulate an activity, such as induction, strength or duration of sodium channel current associated with hASIC2b subunit-containing ion channel activity. Such assays generally comprise contacting a cell that expresses a hASIC2b subunit-containing sodium channel with a test compound. The cell should preferably be contacted with the test compound before or during exposure of an agent or stimulus (e.g., a drop in extracellular pH) that otherwise activates (i.e., opens) the hASIC2b subunit-containing ion channel. The cell can then be examined to determine whether a response otherwise associated with hASIC2b subunit-containing channel activation has been inhibited. In one non-limiting embodiment, the response of the cell treated with the test compound can be compared to a control cell that has not been treated with the test compound. Cell assays include those utilizing conventional, electrode-based electrophysiological techniques, as well as the new generation high-throughput, planar electrode (orifice)-based, electrophysiological technologies, among others. Other assays include monitoring changes in membrane potential with appropriate fluorescent or luminescent dyes, measuring ion flux through the sodium channel with a radiolabeled tracer, or assaying downstream consequences of the sodium channel activation, such as calcium mobilization or effects on gene expression, using an appropriate reporter system.

Positive modulation (i.e., agonism) of hASIC2b subunit-containing channels may be desirable for use in a method to enhance learning and memory, since the hASIC2b-containing channels are expressed in the hippocampus, which is an area of the brain involved in learning and memory. While the mechanisms for such actions are still being elucidated, it appears that local increases in pH at the level of the synapse may decrease the activation threshold of NMDA glutamate (neurotransmitter) receptors. Heavy usage of a synapse by repeated communication between its two neurons strengthens the connection, and strengthening of synapses is one process thought to underlie learning and memory. Further, the enhanced potentiation of NMDA receptors has been demonstrated to be a key step in the activity-dependent plasticity, which is considered to be the biological correlate of learning and memory. Since activation of hASIC2b-containing ion channels enhances activation of NMDA receptors, and therefore, communication between neurons, agonism of hASIC2b-containing channels may contribute to enhanced learning and memory.

Screening

According to the present invention, nucleotide sequences encoding the hASIC2b subunit, and the encoded protein subunit itself, are useful targets to identify drugs that are effective in preventing or alleviating pain associated with the function of the ion channel during conditions such as tissue acidosis and inflammation. Examples of drug candidates include: (i) isolated nucleic acids derived from the gene encoding hASIC2b (e.g., antisense or ribozyme molecules); (ii) small organic molecule compounds that recognize and specifically bind to the hASIC2b subunit protein or to the hASIC2b subunit-containing ion channel; and (iii) peptides or peptide analogs that bind to the hASIC2b subunit protein or to the hASIC2b subunit-containing ion channel. In addition, the nucleotide sequences encoding hASIC2b, and the encoded protein subunit itself, are useful for studying the role of such channels both in pain perception and in physiological and pathological brain functions.

Any screening technique known in the art can be used to screen for agonists or antagonists. The present invention contemplates screens for small molecules and mimics, as well as screens for natural products that bind to and agonize or antagonize hASIC2b-containing ion channels. For example, natural product libraries can be screened using assays of the invention for molecules that agonize or antagonize hASIC2b-containing ion channel activity.

Knowledge of the primary sequence of hASIC2b, and the similarity of that sequence with proteins of known function, can provide an initial lead to inhibitors or antagonists. Identification and screening of modulators is further facilitated by determining structural features of the protein, e.g. using X-ray crystallography, neutron diffraction, nuclear magnetic resonance spectrometry, and other techniques for structure determination. These techniques provide for the rational design or identification of agonists and antagonists.

Another approach uses recombinant bacteriophage to produce large libraries. Using the “phage method” (Scott and Smith, Science, 1990;249:386-390; Cwirla, et al., Proc. Natl. Acad. Sci. USA, 1990;87:6378-6382; Devlin et al., Science, 1990;49:404-406), very large libraries can be constructed (106-108 chemical entities). A second approach uses primarily chemical methods, of which the Geysen method (Geysen et al., Molecular Immunology, 1986;23:709-715; Geysen et al., J. Immunologic Methods, 1987;102:259-274); and the method of Fodor et al. (Science, 1991;251:767-773) are examples. Furka et al. (14th International Congress of Biochemistry 1988, Volume #5, Abstract FR:013; Furka, Int. J. Peptide Protein Res., 1991;37:487-493), Houghton (U.S. Pat. No. 4,631,211) and Rutter et al. (U.S. Pat. No. 5,010,175) generally describe methods to produce a mixture of peptides that can be tested as agonists or antagonists.

In another aspect, synthetic libraries, such as those described in Needels et al., Proc. Natl. Acad. Sci. USA, 1993;90:10700-4; Ohhneyer et al., Proc. Natl. Acad. Sci. USA, 1993;90:10922-10926; Lam et al., PCT Publication No. WO 92/00252; and Kocis et al., PCT Publication No. WO 94/28028, and the like, can be adapted to screen for compounds according to the present invention.

Test compounds can be screened from large libraries of synthetic or natural compounds. Numerous means are currently used for random and directed synthesis of saccharide, peptide, and nucleic acid based compounds. Synthetic compound libraries are commercially available from a variety of sources, including Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Alternatively, libraries of natural compounds in the form of bacterial, fungal, plant and animal extracts are available from a variety of sources including, e.g., Pan Laboratories (Bothell, Wash.) and MycoSearch (N.C.), or are readily producible de novo. Additionally, natural and synthetically produced libraries and compounds are readily modified through conventional chemical, physical, and biochemical means (see, e.g., Blondelle et al., TIBTech 1996, 14:60).

In Vitro Screening Methods

Cell-Based screening

Intact cells expressing a hASIC2b subunit-containing ion channel can be used in screening methods to identify drug candidate compounds useful in modulating the activity of acid sensing ion channels containing the hASIC2b subunit.

In one embodiment, a cell line is established that stably expresses or overexpresses the hASIC2b subunit protein, alone or in combination with other hASIC subunit proteins required to form a functional ion channel, such as hASIC1, hASIC2a, or hASIC3. Alternatively, cells (including without limitation mammalian, insect, yeast, or bacterial cells) are transiently modified to express or overexpress a hASIC2b subunit protein by introduction of appropriate DNA or mRNA.

In another embodiment, human cells wherein the endogenous gene encoding the hASIC2b subunit is not normally expressed, or is expressed at a sub-optimal level, can be manipulated by gene activation techniques to express or overexpress the hASIC2b subunit protein. Identification of candidate compounds can be achieved using any suitable assay, including without limitation: (i) assays that measure binding of test compounds to the hASIC2b subunit-containing ion channel or to the hASIC2b subunit itself; (ii) assays that measure the ability of a test compound to modulate (i.e., antagonize or agonize) a measurable activity or function of hASIC2b subunit-containing ion channel; and (iii) assays that measure the ability of a compound to modulate (i.e., inhibit or enhance) the transcriptional activity of sequences derived from the promoter (i.e., regulatory) regions of the hASIC2b gene.

The screening assays described herein may also comprise the comparison of ion channel activity or other measurable activity related to hASIC2b, to a control. Suitable controls include, but are not limited to, the ion channel activity or other measurable activity in the absence of compound, the ion channel activity or other measurable activity before addition of the test compound, or another experimentally or theoretically determined control value.

hASIC2b Activity Assays

Any cell assay system that allows for assessment of functional activity of hASIC2b subunit-containing ion channels is encompassed by the present invention. In a specific embodiment, exemplified infra, the assay can be used to identify compounds that selectively interact with the hASIC2b subunit protein or ion channels containing the hASIC2b subunit protein. The interaction can be evaluated by assessing the effects on hASIC2b subunit-expressing cells (i.e., cells expressing appropriate ASIC subunit components, including hASIC2b, that form a functional hASIC2b subunit-containing ion channel) of a test compound, under otherwise ASIC-activating conditions (e.g., low extracellular pH). The assay system can be used to identify compounds that selectively produce a functional effect through modulation of hASIC2b subunit-containing ion channels. Compounds that decrease the ability of the ion channel to respond to extracellular decreases in pH (i.e., antagonists) may be useful as novel therapeutics in the amelioration of nociceptive pain, e.g., resulting from tissue inflammation and acidosis. Additionally, CNS-associated conditions related to hASIC2b activity may be treated. For example, changes in extracellular acidity are produced in the brain during ischemia and epileptic seizures, which are other mechanisms by which the ASIC-type channels may be activated.

In one embodiment, proton-gated cation channel-containing cells may be subjected to the action of protons by adjustment of the pH to an acidic level using any convenient acid or buffer, including organic acids such as formic acid, acetic acid, citric acid, ascorbic acid or lactic acid, and inorganic acids such as hydrochloric acid, hydrobromic acid, nitric acid, perchloric acid or phosphoric acid. The current flux of ions induced by protons over the membrane of the proton-gated cation channel-containing cell may be monitored by patch clamp techniques.

In a particular type of screening assay, the ion flux over the membrane of a host cell genetically engineered to express or overexpress a hASIC2b-containing acid-sensitive ion channel is measured in the presence of a test compound, and compared to a control. The control may be, for example, the ion flux prior to the addition of the test compound but under otherwise identical conditions; the ion flux over the membrane of a host cell of the same type as the first host cell and genetically engineered in the same manner, but to which no test compound, or a reference compound, has been added; or another suitable control or control value.

Alternatively, the change in membrane potential induced by protons of the proton-gated cation channel-containing cells may be monitored using fluorescence methods. When using fluorescence methods, the proton-gated cation channel-containing cells are incubated with a membrane potential indicating agent that allows for a determination of changes in the membrane potential of the cells caused by the added protons. Such membrane potential indicating agents include fluorescent indicators, preferably DIBAC4(3), DiOC5(3), and DiOC2(3), among others. If desired, the same type of controls can be used for fluorescence methods as those described for ion flux measurements.

Another method that allows for assessment of functional activity of hASIC2b subunit-containing ion channels involves monitoring the change in membrane potential induced by protons on the proton-gated cation channel-containing cells by spectroscopic methods, e.g. using a FLIPR assay (Fluorescence Image Plate Reader; available from Molecular Devices, Sunnyvale, Calif.). Also, see above section entitled “Agonists and Antagonists.”

High-Throughput Screen

Drug candidates according to the invention may be identified by screening in high-throughput assays, including without limitation cell-based or cell-free assays. It will be appreciated by those skilled in the art that different types of assays can be used to detect different types of drug candidates. Several methods of automated assays have been developed in recent years so as to permit screening of tens of thousands of test compounds in a short period of time. Such high-throughput screening methods are particularly preferred. The use of high-throughput screening assays to test for drug candidates is greatly facilitated by the availability of large amounts of purified polypeptides, as provided by the invention

Published PCT application WO 01/81369, herein incorporated by reference, discloses an array assay for determining agonists, antagonists, or modulators for acid-sensing ion channels, which can be adapted for the screening methods of the present invention. The assay is especially useful for screening for analgesic compounds.

Therapeutic Uses

According to the present invention, inhibition of hASIC2b subunit-containing channel activity may be used as a treatment option in patients with nociceptive pain-related disease states. The upregulation of ASICs, along with the tissue acidosis known to accompany inflammation make them prime targets for novel therapeutics. Inhibition of hASIC2b subunit-containing ion channel activity may be accomplished by methods, such as, but not limited to: (i) providing nucleic acids that inhibit activity; (ii) providing polypeptides that inhibit channel activity; and (iii) providing small organic molecules that inhibit channel activity.

hASIC2b expression or activity may be modulated for therapeutic purposes. Gene transcription and protein translation may be inhibited by administration of a compound. Such a compound may interact with extracellular and/or intracellular messenger systems to regulate protein synthesis. In this embodiment, such a compound that inhibits hASIC2b protein synthesis may be used in the prevention and/or treatment of pain resulting from inflammation or tissue acidosis, or constitutive ion channel activity associated with ischemic or epileptic seizures. Such a compound can be an antisense or ribozyme molecule.

Alternatively, the modulatory method of the invention involves contacting a cell, tissue or subject with an agent that modulates one or more of the activities of the hASIC2b subunit protein or hASIC2b subunit-containing channel associated with the cell. Such an agent can be an agent as described herein, such as a nucleic acid or a protein, an hASIC2b subunit-specific antibody, an hASIC2b agonist or antagonist, a peptidomimetic of an hASIC2b agonist or antagonist, or a small organic molecule. In one embodiment, the agent stimulates one or more hASIC2b subunit activities or one or more hASIC2b subunit-containing channel activities.

Such modulatory methods can be performed in vitro (e.g., by culturing the cell with the agent) or, alternatively, in vivo (e.g., by administering the agent to a subject). As such, the present invention provides methods for treating an individual afflicted with a disease or disorder characterized by aberrant or unwanted expression or activity of a hASIC2b subunit protein or nucleic acid molecule. In one embodiment, the method involves administering an agent (e.g., an agent identified by a screening assay described herein), or combination of agents, that downregulates hASIC2b subunit expression or antagonizes hASIC2b subunit-containing channel activity.

In an alternative embodiment, the agent enhances one or more hASIC2b subunit activities or one or more hASIC2b subunit containing channel activities, such as by administering an hASIC2b protein or nucleic acid molecule as therapy to compensate for reduced or aberrant hASIC2b expression or activity.

Antisense nucleic acids and ribozymes or the present invention may be used to inhibit expression of hASIC2b nucleotide sequences of the invention. Antisense nucleic acid molecules can be encoded by a recombinant gene for expression in a cell (e.g., U.S. Pat. No. 5,814,500; U.S. Pat. No. 5,811,234) or can be prepared synthetically (e.g., U.S. Pat. No. 5,780,607). Ribozymes are described further in Intracellular Ribozyme Applications: Principals and Protocols, Ed. Rossi and Couture, 1999, Horizon Scientific Pr. Alternatively, RNAi or siRNA technology can be used as a therapeutic approach.

Alternatively, antibody molecules can be administered either directly or indirectly or by expressing nucleotide sequences encoding antibodies or binding fragments thereof within the target cell population by utilizing, for example, techniques such as those described in Marasco et al. (Proc. Natl. Acad Sci. USA, 1993;90:7889-7893).

Formulations and Administration

The agent that modulates hASIC2b activity is advantageously formulated in a pharmaceutical composition, by admixing with a pharmaceutically acceptable carrier. This agent may be called an “active ingredient”, or “therapeutic agent”, useful, for example against pain associated with tissue acidosis and inflammation.

The concentration or amount of the active ingredient or agent depends on the desired dosage and administration regimen, as discussed below. Suitable dose ranges may include from about 1 mg/kg to about 100 mg/kg of body weight per day.

The pharmaceutical compositions may also include other biologically active substances in combination with the hASIC2b subunit-containing channel modulatory agent. Such substances include, e.g., opioids such as, e.g., morphine, codeine, fentynyl, oxycodone, hydrocodone, and buprenorphine, among others, and non-steroidal anti-inflammatory drugs (NSAID's) such as, e.g., ibuprofen and COX-2 inhibitors, among others.

The phrase “pharmaceutically acceptable” refers to molecular entities and compositions that are physiologically tolerable and do not typically produce an allergic or similar untoward reaction, such as gastric upset, dizziness and the like, when administered to a human. As used herein, the term “pharmaceutically acceptable” preferably means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the agent is administered. Such pharmaceutical carriers can be sterile liquids, such as aqueous solutions and oils, including those of petroleum, animal, vegetable or synthetic origin, such as peanut oil, soybean oil, mineral oil, sesame oil and the like. Water, or aqueous saline solutions, aqueous dextrose, and glycerol solutions are preferably employed as carriers, particularly for injectable solutions. Suitable pharmaceutical carriers are described in “Remington's Pharmaceutical Sciences” by E. W. Martin.

Therapeutically effective compounds may be provided to the patient in formulations that are known in the art and may include any pharmaceutically acceptable additives, such as excipents, lubricants, diluents, flavorants, colorants, buffers and disintegrants. The formulations may be produced in useful dosage units such as tablet, caplet, capsule, liquid, or injection.

The form, amount and route of administration of the therapeutic compound envisioned for use depends on the type and severity of the disease or condition to be treated, as well as the patient's state of health, gender, weight, age, etc., and can be determined by an attending medical practitioner in view, e.g., of the results of published clinical trials. The formulation may be adopted for oral, intranasal, parenteral (e.g., intravenous, intramuscular, or subcutaneous etc.), or topical (e.g., salve, cream, lotion, gel, or spray, etc.) administration, and may provide for immediate, sustained, or controlled release of the agent to the patient.

### EXAMPLES

The present invention is further described by means of the example, presented below. The use of such an example is illustrative only and in no way limits the scope and meaning of the invention or of any exemplified term. Likewise, the invention is not limited to any particular preferred embodiments described herein. Indeed, many modifications and variations of the invention will be apparent to those skilled in the art upon reading this specification and can be made without departing from its spirit and scope. The invention is therefore to be limited only by the terms of the appended claims along with the full scope of equivalents to which the claims are entitled.

### Example 1

### Cloning of Human ASIC2b (hASIC2b)

Methods

Database screening. The National Center for Biotechnology Information (NCBI) Human Genome Database was searched with the rat MDEG2 (rat ASIC2b; described in GenBank Accession No. Y14635) nucleotide sequence using the BLASTN and TBLASTN algorithms. Hits from the search with alignments to the Homo sapiens chromosome 17 working draft sequence (described in GenBank Accession No. NT_{—}010679.3|Hs17_{—}10836) segments were pieced together in an arrangement resembling that of the rat MDEG2 sequence. Coding regions of human ASIC2b (SEQ ID NO: 1), including the likely start and stop codons, were identified by comparison to the rat MDEG2 sequence.

Reverse transcription and amplification of hASIC2b CDNA. Human RNA was purchased from Clontech (Palo Alto, Calif.). Reverse transcription was carried out using ThermoScript Reverse Transcriptase (Life Technologies, Rockville, Md.), at an annealing temperature of 55° C. to maximize the likelihood of obtaining a full-length mRNA. ThermoScript Reverse Transcriptase is an avian Rnase H(−) reverse transcriptase that functions optimally at 50-65° C. compared to the 37-43° C. temperature range of other reverse transcriptases. The higher temperature helps decrease secondary structure formation in the mRNA during reverse transcription and produces higher yields and a higher percentange of full-length cDNAs than other reverse transcriptases.

The following primers were designed to amplify the resulting full-length hASIC2b cDNA:

| | | |
|---|---|---|
| Forward primer: | 5′-(G)GAATGAGCCGGATTGGCGGAG-3′ | (SEQ ID NO: 12) |
| Reverse primer: | 5′-GAGGGGTGTCAGCAGGCAATCTC-3′ | (SEQ ID NO: 13) |

cDNA was amplified using the Advantage-GC 2 PCR Kit (Clontech, Palo Alto, Calif.) according to the manufacturer's instructions. Sequences that are GC-rich possess strong secondary structure that resist denaturation and impede primer annealing during PCR. The Taq polymerase of the Advantage-GC Kit has properties that reduce or eliminate non-specific amplification products, and destabilizes DNA secondary structure by weakening base-pairing in GC-rich regions.

Purification and cloning of PCR products into expression vectors. PCR products resulting from the above-described reaction were gel-purified using the SNAP UV-Free Gel Purification Kit (InVitrogen, Carlsbad, Calif.) according to the manufacturer's instructions. DNA was stored in Tris-EDTA buffer, pH 7.4.

The PCR products were cloned into the commercially available mammalian expression vector pEF6/V5-HIS TOPO using the pEF6N5-HIS TOPO TA Expression Kit (InVitrogen, Carlsbad, Calif.). Once cloned, the vectors were transformed into One Shot TOP 10 competent E. coli bacteria, provided in the kit according to manufacturer's instructions. Bacterial expressing hASIC2b were grown at 33° C. instead of the traditional 37° C. to decrease the growth rate and minimize stress on the bacterial that could result in mutations to the cDNA, or activation of the pH-sensitive acid channels.

Ampicillin-resistant colonies were selected, grown-up, and the cDNA was extracted using the Wizard Plus SV Minipreps DNA Purification System. Kit (Promega, Madison, Wis.) according to the manufacturer's instructions. cDNA was then analyzed by restriction digest, and three clones with the correct insertion orientation and restriction fragment sizes were selected (SC2, FB4 and CB12). The Wizard Plus DNA MidiPreps DNA Purification System (Promega, Madison, Wis.) was then used to prepare a greater quantity of cDNA for further use and analysis.

Sequencing of cDNA clones. The three cDNA clones (CB12, FB4, and SC2) were sequenced by MWG Biotech (High Point, N.C.). Results are presented in SEQ ID NO:6, SEQ ID NO:8, and SEQ ID NO:10, respectively. The adenosine (a) of the start codon ATG is located at residue 3, 3, and 4, of SEQ ID NOS: 6, 8, and 10, respectively. See also SEQ ID NOS: 15-17.

In vitro transcription and translation and detection. Purified hASIC2b protein was generated from the purified cDNA using the TNT Quick Coupled Transcription/Translation System with Transcend™ (Promega, Madison, Wis.), according to the manufacturer's instructions. The translated protein products were separated on a NuPAGE 4-12% Bis-Tris gel (InVitrogen, Carlsbad, Calif.), in the presence of NuPAGE Reducing Agent (InVitrogen, Carlsbad, Calif.). The molecular weight of the separated proteins was approximated using the MultiMark Multi-Colored Standard (InVitrogen, Madison, Wis.).

The separated proteins were transferred to a PVDF Transfer Membrane (Amersham, Piscataway, N.J.) in standard transfer buffer (25 mM Tris, 0.01% Sodium Dodecyl Sulfate, 0.19 M glycine, 20% methanol), overnight at 4° C. Proteins were visualized using the Transcend™ Non-Radioactive Chemiluminescent Translation Detection System (Promega, Madison, Wis.).

Results

The predicted hASIC2b genomic nucleotide sequence (SEQ ID NO:1) has about 96% sequence identity to that encoding the rat ASIC2b (not shown). The nucleotide sequence of hASIC2b shares about 70-75% sequence identity with the hASIC2a sequence of Price (described in GenBank Acc. No. NM_{—}001094; SEQ ID NO: 3; J. Biol. Chem., 1997;272(47):29778-29783). Notably, the novel hASIC2b lacks 280 base pairs corresponding to bases 724 to 1004 of the rat sequence.

cDNA nucleotide sequences of three clones of hASIC2b cDNA generated from human cerebellar, fetal brain, and spinal cord are depicted in SEQ ID NOs: 6, 8, and 10, respectively, along with their corresponding deduced amino acid sequences (shown in SEQ ID NOs: 7, 9, and 11, respectively). The hASIC2b cerebellum cDNA clone, designated CB12 (SEQ ID NO:6), comprises 1607 nucleotides, which differs from that of hASIC2a (described in GenBank Acc. No. NM_{—}001094, SEQ ID NO: 4) beginning from nucleotides 1 to 708. The remaining nucleotides (709-1702) are substantially identical to hASIC2a (SEQ ID NO:4). The amino acid sequence of CB12 (SEQ ID NO: 7) differs significantly from hASIC2a from amino acid residues 1-236, with the remaining amino acid residues being substantially identical to hASIC2a.

The hASIC2b fetal brain cDNA clone, designated FB4 (SEQ ID NO:8), differs from hASIC2a from bases 1-710. Bases 711-1702 are identical to the corresponding regions of hASIC2a. Similarly, the corresponding amino acid sequence of FB4 (SEQ ID NO:9) is substantially different from amino acid residues 1-236 but is identical to hASIC2a from 237 to 563, with the exception of residues 287 and 503 (E to K, and D to G, respectively).

Similar to the CB12 and FB4, the hASIC2b spinal cord tissue clone, designated SC2 (SEQ ID NO:10), differs from hASIC2a in nucleotides 1-708, but is substantially identical to hASIC2a from nucleotides 709-1702. Accordingly, the first 236 residues in the amino acid sequence (SEQ ID NO:11) differ from hASIC2a, but the remaining sequence is substantially similar to hASIC2a beginning from amino acid residue 237 to the end, with the exception of residue 483 (I replaces M).

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Patents, patent applications, publications, procedures, and the like are cited throughout this application, the disclosures of which are incorporated herein by reference in their entireties.

What is claimed is:

1. An isolated nucleic acid comprising a nucleotide sequence encoding a polypeptide having an amino acid sequence at least 95% identical to SEQ ID NO:14.

2. The isolated nucleic acid of claim 1, comprising a nucleotide encoding a polypeptide having the amino acid sequence of SEQ ID NO:14.

3. The isolated nucleic acid of claim 2, comprising SEQ ID NO:1.

4. The isolated nucleic acid of claim 2, comprising a nucleotide sequence encoding a polypeptide having the amino acid sequence of SEQ ID NO:7.

5. The isolated nucleic acid of claim 4, comprising SEQ ID NO:15.

6. The isolated nucleic acid of claim 2, comprising a nucleotide sequence encoding a polypeptide having the amino acid sequence of SEQ ID NO:9.

7. The isolated nucleic acid of claim 6, having the nucleotide sequence of SEQ ID NO:16.

8. The isolated nucleic acid of claim 2 comprising a sequence encoding a polypeptide having the amino acid sequence of SEQ ID NO:11.

9. The isolated nucleic acid of claim 8, comprising SEQ ID NO:17.

10. A recombinant vector comprising the nucleic acid of claim 1.

11. A host cell comprising the recombinant vector of claim 10, wherein the nucleic acid is operatively associated with an expression control sequence.

12. The host cell of claim 11, which is a eukaryotic host cell.

13. A method for expressing a polypeptide in a cell cultured in vitro, comprising culturing the cell of claim 11 under conditions allowing expression of a human acid-sensing ion channel 2b (hASIC2b) polypeptide encoded by the nucleic acid.

14. A eukaryotic host cell genetically engineered to express or overexpress a hASIC2b polypeptide having the amino acid sequence of SEQ ID NO:14.

15. The eukaryotic host cell of claim 15, wherein said host cell further expresses a protein selected from the group consisting of hASIC1, hASIC3, hASIC4, and combinations thereof.

16. A method for expressing a polypeptide in a cell cultured in vitro, comprising culturing the cell of claim 14 under conditions allowing expression of the hASIC2b from a nucleic acid operatively associated with an expression control sequence.

17. An isolated polypeptide comprising an amino acid sequence at least 95% identical to SEQ ID NO:14.

18. The isolated polypeptide of claim 17, comprising SEQ ID NO:14.

19. The isolated polypeptide of claim 18, comprising the amino acid sequence of SEQ ID NO:7.

20. The isolated polypeptide of claim 18, comprising the amino acid sequence of SEQ ID NO:9.

21. The isolated polypeptide of claim 18, comprising the amino acid sequence of SEQ ID NO:11.

22. An antibody or antigen-binding antibody fragment that specifically binds to a polypeptide having a sequence selected from the group consisting of SEQ ID NO:7, SEQ ID NO:9, SEQ ID NO:11, and SEQ ID NO:14.

23. The antibody of claim 22, which is a monoclonal antibody.

24. A method for detecting a hASIC2b polypeptide, which method comprises detecting binding of an antibody or antigen-binding antibody fragment of claim 22 to the polypeptide in a sample.

25. A method for identifying a compound that binds to a hASIC2b polypeptide, which method comprises:
(i) contacting a hASIC2b polypeptide having the amino acid sequence of SEQ ID NO:14, or a fragment thereof, with a test compound;
(ii) determining whether the test compound binds to the hASIC2b polypeptide or fragment thereof; and
(iii) identifying any compound that binds to the hASIC2b polypeptide or fragment thereof.

26. The method of claim 25, wherein the hASIC2b polypeptide is expressed by a host cell.

27. The method of claim 25, wherein the hASIC2b polypeptide is in a cell extract or a membrane fraction.

28. A method for identifying a host cell that expresses a hASIC2b polypeptide, which method comprises:
(i) contacting a test host cell with an oligonucleotide complementary to an mRNA sequence encoding a hASIC2b polypeptide having the amino acid sequence of SEQ ID NO:14; and
(ii) observing whether the oligonucleotide binds to an mRNA sequence transcribed by the test host cell.

29. An assay method for screening for a test compound that modulates hASIC2b subunit-containing ion channels, which method comprises:
(i) contacting a host cell that has been genetically engineered to express or overexpress a functional acid sensing ion channel (ASIC) comprising at least one hASIC2b subunit protein with a test compound under conditions that would otherwise activate ion channel activity of said functional ASIC; and
(ii) comparing the ion channel activity in the presence of the test compound to a control.

30. The assay method of claim 29, wherein the control is the ion channel activity of the host cell in the absence of test compound and under conditions that would otherwise activate ion channel activity.

31. The assay method of claim 29, wherein the control is the ion channel activity of a second host cell of the same type in the absence of test compound and under conditions that would otherwise activate ion channel activity.

32. The assay method of claim 29, wherein the host cell has been genetically engineered to express or overexpress a hASIC2b-encoding nucleic acid molecule endogenous to said host cell.

33. The assay method of claim 32, wherein the host cell has been genetically engineered to express or overexpress endogenous nucleic acid molecules encoding the functional acid sensing ion channel comprising the hASIC2b subunit protein.

34. The assay method of claim 29, wherein the host cell has been genetically engineered by the introduction into the host cell of a nucleic acid molecule having a nucleotide sequence encoding the hASIC2b subunit protein and being operatively associated with an expression control sequence.

35. The assay of claim 29, wherein said modulation of said functional acid sensing ion channel activity is antagonism of said activity.

36. The assay of claim 29, wherein said modulation of said functional acid sensing ion channel activity is agonism of said activity.
